(19) United States
(12) Patent Application Publication
(10) Pub. No.: US 2026/0273288 A1
(43) Pub. Date: Sep. 17, 2026

(54) SYSTEM AND METHODS FOR DETERMINING AND TUNING DETECTION SETTINGS IN A NEUROSTIMULATOR

(71) Applicant: NeuroPace, Inc., Mountain View, CA (US)

(72) Inventors: David A. Greene, Fort Wayne, IN (US); Thomas K. Tcheng, Pleasant Hill, CA (US); Muhammad Furqan Afzal, Forest Hills, NY (US); Jacob F. Norman, South Boston, MA (US); Jatin Sharma, San Jose, CA (US); Brett M. Wingeier, San Francisco, CA (US)

(21) Appl. No.: 19/677,894

(22) Filed: May 14, 2026

Related U.S. Application Data
(63) Continuation-in-part of application No. 19/214,456, filed on May 21, 2025.
(60) Provisional application No. 63/650,504, filed on May 22, 2024.
(60) Provisional application No. 63/806,274, filed on May 15, 2025.

(51) Int. Cl. A61N 1/372; A61B 5/00; A61B 5/374; A61N 1/36

(52) U.S. Cl. CPC A61N 1/37235; A61B 5/374; A61B 5/4094; A61B 5/4836; A61B 5/7221; A61B 5/7264; A61N 1/36064; A61N 1/36139

(57) ABSTRACT

A system manages the configuration of an implanted neurostimulation system having an adaptive phase of operation during which stimulation therapy is not delivered. The system determines an initial detection tool for the implanted neurostimulation system while the system is in the adaptive phase. The system performs at least one tuning cycle of the initial detection tool while the system is still in the adaptive phase. To this end, the system adjusts a detection parameter of the initial detection tool; receives data from the neurostimulation system that results from detections of an onset pattern by the adjusted initial detection tool for a period of time subsequent to the adjustment of the detection parameter; obtains a performance metric for the adjusted initial detection tool based on the data; determines if the performance metric satisfies a target performance metric; and responsive to the target performance metric not being satisfied, performs another tuning cycle.

### CROSS-REFERENCE TO RELATED APPLICATIONS

This application claims the benefit of U.S. Provisional Application Ser. No. 63/806,274, titled “Systems and Methods for Automatically Tuning Settings in a Neurostimulator,” filed on May 15, 2025, and is a continuation-in-part of U.S. application Ser. No. 19/214,456, titled “System and Methods for Selecting Detection Parameters for Detecting Epileptiform Activity,” filed on May 21, 2025, which claims the benefit of U.S. Provisional Application Ser. No. 63/650,504, titled “System and Methods for Selecting Detection Parameters for Detecting Electrographic Seizures,” filed on May 22, 2024, each of which is expressly incorporated by reference herein in its entirety.

### TECHNICAL FIELD

The present disclosure relates generally to the adjustment of treatment for a neurological disorder, and more particularly, systems, devices, and methods for adjusting settings of a neurostimulator for treatment of a neurological disorder, in which settings that govern detection of brain activity that is desirable to treat (such as epileptiform activity) are adjusted automatically based on biosignals or other data.

### BACKGROUND

Systems and methods that include algorithms for detecting when physiological data sensed from a patient exhibit certain features or correspond to certain physiological states are desirable in diagnosing, monitoring and treating patients. For example, systems and methods that include algorithms for detecting epileptiform activity, which may include preictal epileptiform activity, post-ictal epileptiform activity, inter-ictal epileptiform activity, and/or electrographic seizures, are desirable in diagnosing, monitoring, and treating patients with epilepsy. Specifying the parameters necessary for these algorithms to operate as expected and to generate the desired outcome is generally not an intuitive process for the patient's physician. It would be beneficial to make these systems and methods easier for a physician, clinician, clinical engineer, or other individual involved in the treatment of patients with epilepsy to use with regard to a particular patient or set of patients.

Brain stimulation has emerged as a highly effective technique for treating a range of neurological disorders, including Parkinson's disease and epilepsy. Devices such as NeuroPace's RNS System for epilepsy and Medtronic's DBS for Parkinson's disease have received FDA approval and are now in clinical use. In addition to delivering therapeutic stimulation, some of these devices are capable of recording brain activity, which is particularly valuable for understanding the brain's response to stimulation. Analyzing brain activity before, during, and/or after pathological activity can provide crucial insights into how to optimize stimulation to effectively treat a disease or disorder.

Devices are now being used as closed-loop neurostimulators in patients with epilepsy and other neurological disorders. Such closed loop neurostimulators sense and react to abnormal electrical brain activity that is highly heterogeneous in nature. Examples of these abnormal patterns are gamma bursts, spikes, sharp waves, high amplitude activity, and spike/wave activity. Because of the heterogeneous nature of the abnormal signals, the detection settings need to be customized for each patient. This is presently done by expert reviewers examining electrocorticograms (ECoGs) and detection rates to assess performance, and then by programming manual adjustments in office visits. Currently, it will often take several visits to adjust the detection settings adequately, with the result that there is a significant time between the initial implant and the neurostimulator and the time when detections settings are determined to be adequately configured. In some cases, detection is not adequately configured, leading to sub-optimal treatment.

Given these limitations, there is a need for more automated approaches to adjustment of detection settings. The methods and systems disclosed herein fulfill this need.

### SUMMARY

This disclosure relates to a system that manages the configuration of an implanted neurostimulation system having a therapy phase of operation during which stimulation therapy is delivered, and an adaptive phase of operation during which stimulation therapy is not delivered. The system includes a patient data management system (PDMS) configured to obtain an initial detection tool for the implanted neurostimulation system while the neurostimulation system is in the adaptive phase. The initial detection tool is configured to detect an onset pattern in electrographic activity sensed by a sensing channel of the implanted neurostimulation system. The initial detection tool is defined by a set of detection parameters. The PDMS is also configured to perform at least one tuning cycle of the initial detection tool while the neurostimulation system is still in the adaptive phase to determine a tuned initial detection tool. To this end, the PDMS is configured to adjust a detection parameter of the initial detection tool; receive for a period of time subsequent to the adjustment of the detection parameter, data from the implanted neurostimulation system that results from detections of the onset pattern by the adjusted initial detection tool; obtain a performance metric for the adjusted initial detection tool based on the data; determine if the performance metric satisfies a target performance metric; responsive to the target performance metric not being satisfied, perform another tuning cycle; and responsive to the target performance metric being satisfied, provide configuration information that defines the tuned initial detection tool, where the provided configuration information corresponds to the configuration information of the adjusted initial detection tool that satisfied the target performance metric.

This disclosure also relates to a method of managing the configuration of an implanted neurostimulation system. The method includes enabling an adaptive phase of an implanted neurostimulation system during which the implanted neurostimulation system refrains from delivering stimulation therapy; and during a first period of time of the adaptive phase, obtaining an initial detection tool configured to detect an onset pattern in electrographic activity sensed by a sensing channel of the implanted neurostimulation system, where the initial detection tool is defined by a set of detection parameters. The method further includes during a second period of time of the adaptive phase, performing at least one tuning cycle for the initial detection tool to determine a tuned initial detection tool. A tuning cycle includes adjusting a detection parameter of the initial detection tool; for a period of time subsequent to the adjustment of the detection parameter, detecting for the onset pattern in electrical activity using the adjusted initial detection tool, where the electrical activity is sensed during the period using the implanted neurostimulation system; obtaining a performance metric for the adjusted initial detection tool based on data that results from the detecting; determining if the performance metric satisfies a target performance metric; responsive to the target performance metric not being satisfied, performing another tuning cycle; and responsive to the target performance metric being satisfied, providing configuration information that defines the tuned initial detection tool. The method also includes enabling a therapy phase of the implanted neurostimulation system during which neurostimulation therapy is delivered in response to a detection of the onset pattern by the tuned initial detection tool.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1A are example time-series waveform visual representations of records of electrical activity, e.g., electroencephalogram (EEG) records, corresponding to electrical activity of the brain recorded by an implanted neurostimulation system, together with their corresponding spectrogram visual representations.

FIG. 1B are example time-series waveform visual representations of EEG records, together with additional information, e.g., time stamps and seizure/non-seizure labels, associated with the records and included in a patient dataset.

FIG. 1C are example time-series waveform visual representations of EEG records corresponding to patterns of electrical brain activity including spikes, oscillatory patterns, and amplitude and/or frequency changes.

FIG. 2 is a block diagram illustration of a system, including an implanted neurostimulation system, a detection proposer, and other external equipment, that enables automatic determination and adjustment of parameter sets for detection tools of the neurostimulation system based on EEG records recorded by the neurostimulation system.

FIG. 3 is a perspective, schematic illustration of an implanted neurostimulation system implanted in a patient and configured to sense electrical activity of the brain, detect electrographic events through detection tools, and deliver therapy responsive to detections of such events.

FIG. 4 is a block diagram of the implanted neurostimulation system of FIG. 3, illustrating some of the functional subsystems of the system.

FIGS. 5A and 5B is a block diagram illustration of the detection proposer of FIG. 2 that includes an EEG records selection module, a detection window module, a candidate detection tool module, and a detection tool selection module.

FIGS. 6A-6H are further illustrations of the modules of the detection proposer of FIGS. 5A and 5B.

FIGS. 7A, 7B, 7C, and 7D are illustrations of EEG records representing different activity types determined by the detection proposer.

FIGS. 8A-8D is a flow chart of a method of proposing and adjusting a detection tool for an implanted neurostimulation system using the detection proposer of FIGS. 5A and 5B.

FIG. 9A is a block diagram of a training dataset for training a machine learning based model of the detection proposer.

FIG. 9B is a block diagram of an application of the trained machine learning based model of FIG. 9A to determine candidate detection tools.

FIG. 10 is an illustration of detection tool information and a user interface presented on a display by the detection proposer of FIG. 2.

FIG. 11 is a schematic block diagram of an apparatus corresponding to the detection proposer of FIG. 2.

FIG. 12 is an illustration of a system that includes an implantable closed-loop neurostimulator, physician programmer, patient remote monitor, and patient data management system.

FIG. 13 is an illustration of a two-stage method for automatic proposal and adjustment of detection settings for detection tools.

FIG. 14 is a flow chart for the first stage of the method of FIG. 13.

FIG. 15 is an illustration of an intracranial EEG record containing a seizure on two of four channels.

FIG. 16 is an illustration of two intracranial EEG records, one showing a seizure originating from the left side of the brain and one showing a seizure originating from the right side of the brain.

FIG. 17 is an illustration of an output of an exemplary clustering algorithm in reduced dimensional space.

FIG. 18 is an illustration of an intracranial EEG record in which a region of interest has been identified for proposal of detection settings for an initial detection tool.

FIG. 19 is an illustration showing the performance of a proposed initial detection tool when applied to an example intracranial EEG record.

FIG. 20 is an illustration of an intracranial EEG record in which a region of interest is identified for proposal of detection settings for an initial detection tool, and an additional region of interest identified for proposal of detection setting for a sustainment detection tool.

FIG. 21 is an illustration of an example metric for assessing performance of an initial detection tool and a sustainment detection tool when applied to an intracranial EEG record.

FIG. 22 is a flow chart for the second stage of the method of FIG. 13.

FIG. 23 is an illustration of an algorithm for adjusting detection settings in order to minimize the difference between an observed detection rate and a target detection rate.

### DETAILED DESCRIPTION

Disclosed herein are methods and systems for automatically proposing and adjusting detection settings (also referred as “detection parameters”) that enable a neurostimulation system to detect an event, e.g., epileptiform activity, electrographic seizures, and/or other signals characteristic of a neurological disorder, in electrical activity of the brain that is sensed by the neurostimulation system. In some embodiments the neurostimulation system is a closed-loop system having a detection subsystem with one or more detection tools programmed with detection settings and a therapy subsystem that is configured to deliver neurostimulation therapy in response to an event detection by a detection tool. These detection tools are also referred to as “detectors” or “event detectors.”

The disclosed method and system pairs the sensing and data recording capabilities of a closed-loop neuromodulation system with remote data processing and programming features to determine and tune detection parameters for detection tools. In an exemplary embodiment, detection settings are automatically determined and tuned (or adjusted) in the period of time between implant and stimulation activation in order to optimize a desired outcome measure, such as the number of detections that occur per day. This period of time is split into two stages that include a first stage in which detection parameters are first determined for detection tools and coarsely adjusted, and a second stage in which these detection parameters are further adjusted in small increments to optimize performance of the detection tools. In some embodiments, the algorithm that executes the two stages is implemented in a cloud-based patient data management system, and a patient remote monitor communicates data and updated detection settings with both the implanted neurostimulator and the patient data management system.

In some embodiments the detection tools available in the neurostimulation system includes a half wave detector, a line length detector, and an area detector. An objective of a half wave detector is to generate an output whenever the power of a portion of a signal falls within a particular frequency range. It is noted generally that, even though a half wave detector can be concerned with the frequency content of a signal, the tool operates in the time domain rather than in the frequency domain. Half wave detectors are also referred to as bandpass detectors.

An objective of the line length detector is to generate an output that corresponds to how much the frequency and/or amplitude of a portion of a signal within a particular time window is varying relative to, for example, a long-term line length trend for that signal. Line length detectors are also referred to as trending detectors. The line length detector is sometimes referred to as a simplification of the fractal dimension of a waveform. The result of the line length detector is meant to correspond to an approximation of the overall power of the signal relative to a trend. For example, the line length detector is meant to “detect” when a portion of a signal in a given time window departs from the trend and exhibits a change in frequency or amplitude swings or both; a change in amplitude or frequency suggests something different is happening in the patient: for example, when the power increases, the line length detector may detect the onset of or a precursor to an electrographic seizure.

An objective of the area detector is to generate an output that corresponds to how much the integral (or area under a curve) of a signal within a particular time window is varying relative to, for example, a long-term area trend for that signal. Area detectors are also referred to as trending detectors. The area detector is sometimes referred to as a representation of the energy of a waveform. As with the line length detector, the area detector is meant to identify conditions when the signal departs significantly from the long-term trend suggesting something undesirable or abnormal is occurring in the patient.

Even though the half wave detector, the line length detector, and the area detector are each deemed to be algorithms of relatively low complexity, there nonetheless can be a significant number and kind of parameters that need to be specified in order for the running of each algorithm to have an optimal result. A given system can be configured so that all or some of the parameters that control how an algorithm will operate (e.g., what sensed physiological data the algorithm will ‘detect’) are programmable.

The number and kind of parameters for a given detection tool can be relatively easy to understand and specify for an engineer or practicing scientist or for someone who otherwise is interested in how the algorithms operate at a detailed level. However, the typical user (e.g., a busy neurologist or a neurosurgeon with many patients) who is tasked with programming or reprogramming the detection tools may not have the time or inclination to develop a comprehensive understanding of what the various parameters are and how each relates to the condition or state of the patient the user wants the implant to monitor and/or treat. These users are better served by a system that automatically derives the parameters for the various tools based on a dataset of electrographic records.

The complexity of selecting parameters and parameter values for a detection tool are illustrated with reference to the half wave detection tool (or half wave detector). A half wave detection tool is specified by numerous parameters, including: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A line length detection tool is specified by numerous parameters, including: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

In the systems and methods disclosed herein, physiological data, e.g., electroencephalogram (EEG) records, acquired from a patient and comprising an electrographic event, e.g., epileptiform activity associated with a seizure onset, are processed automatically to derive a set of parameters and values for the parameters that will be used by one or more detection tools. The intention is that, if the neurostimulator is programmed with the automatically-derived parameter set, then when the relevant detection tool operates on physiological data acquired from the patient in the future, the tool will detect activity of the same electrographic event, e.g., epileptiform activity associated with a seizure onset, if that activity occurs while the tool is being run. The systems and methods therefore automatically specify the detection tool and a set of operating parameters (or detection parameters) for a given detection tool based on EEG records of the patient.

### Datasets/Data Types

As used herein, a “dataset” refers to a collection of information that is used to derive and propose a detection tool. A dataset includes one or more records or files of information from a patient in whom an implantable medical device (IMD), e.g., neurostimulation system 206, is implanted. This information can include physiological information from the patient and non-physiological information related to the patient's environment, device configuration, device operation, demographics, conditions, and therapies. Physiological information is also referred to herein as data or data types, while non-physiological information is referred to as patient features.

With respect to physiological information, in the case of an implanted neurostimulation system, a dataset includes records or files of physiological information corresponding to electrical activity of the brain that is sensed by the system. Hereinafter, electrical activity of the brain is sometimes referred to as electrographic activity or EEG activity, and a physiological record corresponding to electrical activity of a patient's brain is sometimes referred to as an EEG record. It will be understood that EEG includes electrical activity sensed directly from the neural tissue, which sometimes is referred to as electrocorticographic activity, an electrocorticogram, or “ECOG,” or intra-cranial EEG (“iEEG”).

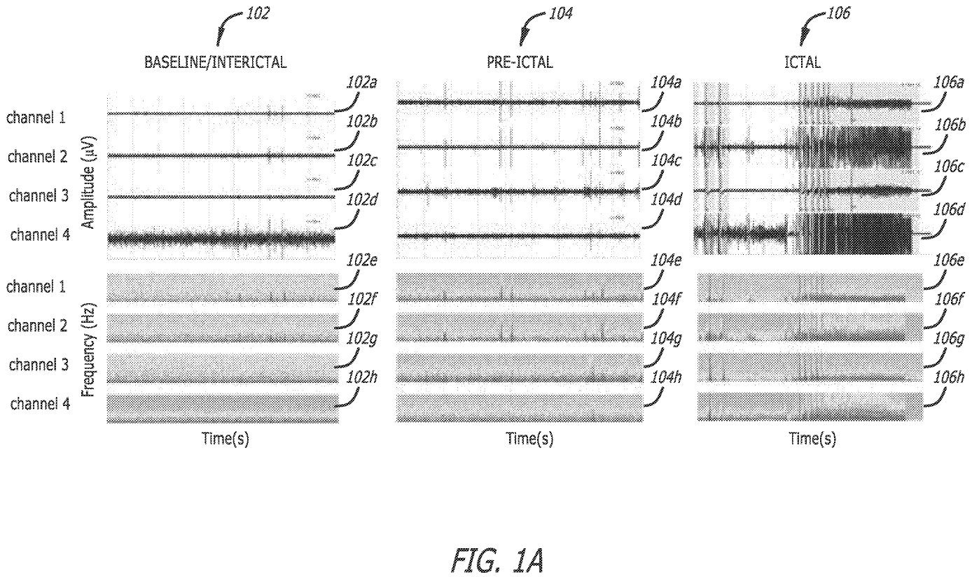

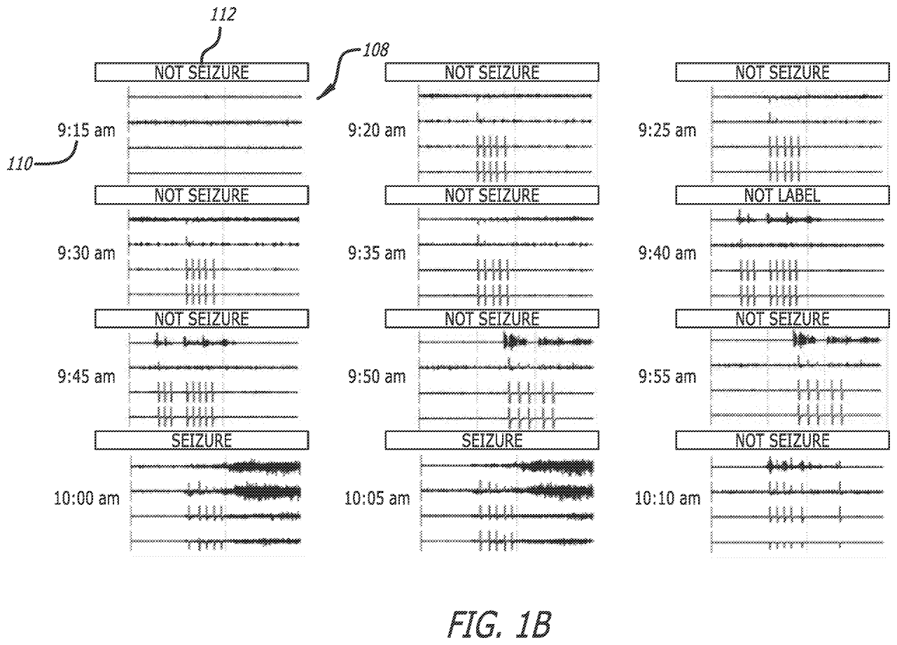

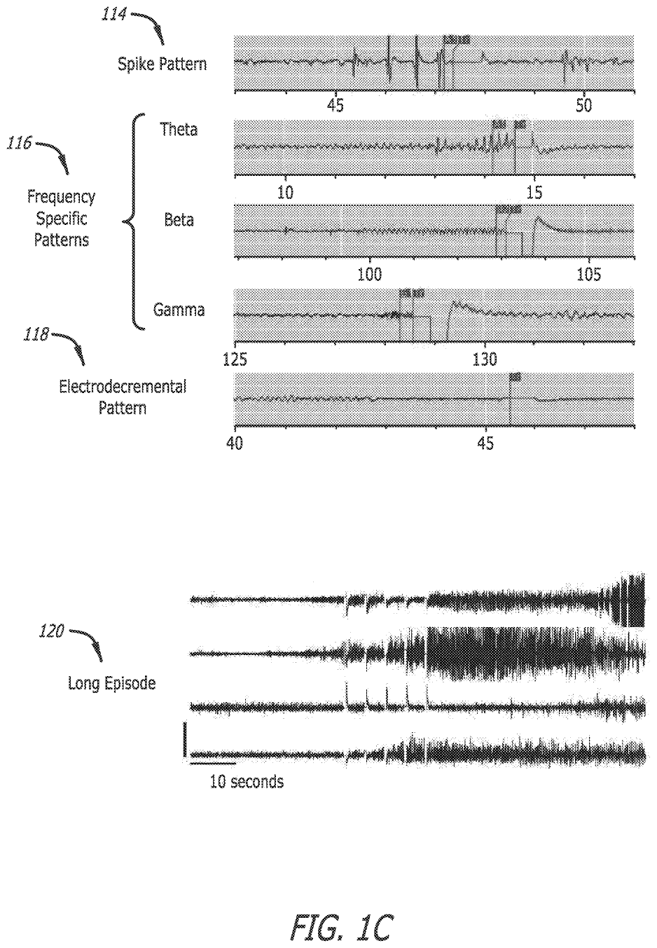

With additional reference to FIG. 1A, EEG records 102, 104, 106 included in a dataset can be visualized or represented in different forms. In the upper portion of FIG. 1A, EEG records 102, 104, 106 are represented by time series waveform images 102a-d, 104a-d, 106a-d for each of four sensing channels of an implanted neurostimulation system. Each EEG record 102, 104, 106 was captured with an implanted neurostimulator system during a respective one of a baseline/interictal brain state (e.g., no seizure), a preictal brain state (e.g., activity captured within the hours before the onset of seizures), and an ictal brain state (e.g., a seizure) in an example patient. In the lower portion, the same EEG records 102, 104, 106 are represented by spectrograms 102e-h, 104e-h, 106e-h for each of the four sensing channels.

With reference to FIG. 1B, additional information or data type can be associated with EEG records. For example, each individual EEG record 108 can have an associated time stamp 110 corresponding to the time the EEG signals within the record were captured by the implanted neurostimulation system. Each individual EEG record 108 can also have an associated label 112 classifying the EEG signals within the record as being indicative of a seizure or not a seizure. Other examples of additional information that can be associated with each EEG record include the event that triggered the creation of the EEG record. As described further below, such triggering events can include a detection of abnormal electrical activity in an EEG signal, a patient-initiated event, e.g., a swipe of a magnet in the area of the implanted neurostimulation system, or a scheduled passage of time.

Additional information or data types can be derived by an implanted neurostimulation system from sensed EEG signals and included in a dataset. For example, in some embodiments, the implanted neurostimulation system is configured to detect patterns in a patient's electrical brain activity and to maintain records of the timing of detections, the count of the number of detections, and a detection rate. The count of such detections can be included in a dataset, either with or without an EEG record of the detected patterns. With reference to FIG. 1C, example patterns of electrical brain activity include spike patterns 114, oscillatory or frequency specific patterns 116, and electrodecremental patterns 118 that exhibit a brief, usually sudden, decrease in the amplitude of brainwave activity. The implanted neurostimulation system can also be configured to detect abnormal electrical brain activity having a duration that exceeds a specified threshold, and to maintain records of the timing and count of the number of such detections together with information, e.g., time stamps, indicative of the time and duration each detection. This abnormal electrical brain activity is referred to as a “long episode.” An example pattern of a long episode 120 is shown in FIG. 1C. The count of detections of long episodes 120 and the respective duration information of each can be included in a dataset, either with or without EEG records of the detected long episodes.

In some embodiments, the implanted neurostimulation system is configured to derive measures from a patient's electrical brain activity and to maintain records of the measures. For example, the implanted neurostimulation system can measure spectral power in certain frequency bands (example 1-4 Hz band, 4-8 Hz band, 8-12 Hz band, 12-25 Hz band, 25-50 Hz band, 50-90 Hz band and so on) computed in small moving and overlapping time windows such as 128, 256 or 512 milliseconds.

While the methods and systems disclosed herein are primarily described with reference to EEG records, it will be appreciated that other physiological information and non-physiological information can be processed. To this end, other types or modalities of physiological information derived from sources other than EEG records can be included in a dataset. For example, physiological records can include measurements of pH level in neural tissue, blood oxygen levels in neural tissue, blood flow rates, neurotransmitters concentrations in neural tissue, temperatures, heart rates, blood pressures, blood glucose levels, hormones sensed in sweat, skin conductivity, accelerometer/motion recordings, posture, and sleep patterns. In some embodiments, this information is sensed and recorded locally by an implanted neurostimulation system. In some embodiments, this information is sensed remote from the implanted medical device, such as from an external wearable device, and transmitted to the implanted neurostimulation system for local storage.

With respect to non-physiological information or patient features, a dataset can include records or files of the patient's demographics (e.g., age, gender, etc.), the patient's drug regimen (e.g., type of drug, dose, and time of day of dose), and the patient's clinical outcomes, such as the rate of clinical seizures (e.g., as reported in a seizure diary), mood, or questionnaire information. A dataset can also include configuration/operation information of the implanted neurostimulation system. Example configuration/operation information includes detection parameters used by the system to detect patterns in a patient's electrical brain activity, and stimulation parameters that define a stimulation therapy delivered by the system.

Typically, some sort of linkage or mapping among the various types of physiological information is provided in a dataset. To this end, in some embodiments each record has one or more associated tags or parameters. For example, physiological records can have a time stamp that allows a set of physiological records at a given point in time to be located for processing. Physiological records can have a tag that indicates the basis, e.g., seizure detection, magnet swipe, scheduled time of day, for preserving the record. These tags allow a set of physiological records to be selected for processing based on a single criterion or a combination of criteria. Other tags include day of capture, area of the brain at which the electrical activity was captured, basis for record creation (e.g., seizure detection, scheduled, patient initiated), characteristic of the record (e.g., power spectral density of EEG signal prior to stimulation).

Examples of data types that can be included in a patient dataset are listed in Table 1:

TABLE 1

| Data Source | Data Type |
|---|---|
| EEG | counts - long episodes (LE), spikes, electrographic |
| (electrical | seizures, abnormal patterns/events |
| activity of | |
| the brain) | |
| | rates - long episodes (LE), spikes, electrographic |
| | seizures, abnormal patterns/events |
| | durations - long episodes (LE), spikes, electrographic |
| | seizures, abnormal patterns/events |
| | distributions - long train distributions (distribution of |
| | episodes that are >10 seconds) |
| | classifications - ictal or interictal |
| | brain activity type (determined by machine learning |
| | based model) |
| | measures - total spectral power, spectral power in certain |
| | frequency bands (example 1-4 Hz band, 4-8 Hz band, 8-2 |
| | Hz band, 12-25 Hz band, 25-50 Hz band, 50-90 Hz |
| | band, phase amplitude coupling, coherence. |
| | spectrogram images |
| | time-series images |
| | coherograms, coherence in seizure activity |
| | coherograms, coherence in scheduled EEG record activity |
| non-EEG | pH level |
| | blood oxygen, flow rate, pressure |
| | temperature |
| | heart rate |
| | neurotransmitter concentrations |
| | glucose level |
| | skin conductivity |
| | hormones sensed |
| | motion/accelerometer |
| | posture |
| | sleep pattern |

Examples of patient features that can be included in a patient dataset are listed in Table 2:

TABLE 2

| Feature Source | Feature |
|---|---|
| patient | age - current, at epilepsy onset, age at implant, sex, |
| demographics | race, etc. |
| | duration of epilepsy (years) |
| | sex |
| | type of job |
| | geographical location |
| patient diagnoses/ | seizure frequency during pre-implant period |
| treatments | |
| | lobe of epilepsy onset - MTL, neocortical, |
| | MTL + neocortical |
| | brain abnormalities - presence of sclerosis, dysplasia |
| | prior cortical resection (yes/no) |
| | prior vagus nerve stimulation (yes/no) |
| | prior EEG monitoring with intracranial electrodes |
| | (yes/no) |
| | drug regimen - list and dosage of medications |
| | clinical outcomes |
| | seizure diary |
| implanted | setting - detection parameters, stimulation parameters |
| neurostimulation | |
| system | |
| | lead implant location |
| | lead type/number - strip, depth |

### Overview of System

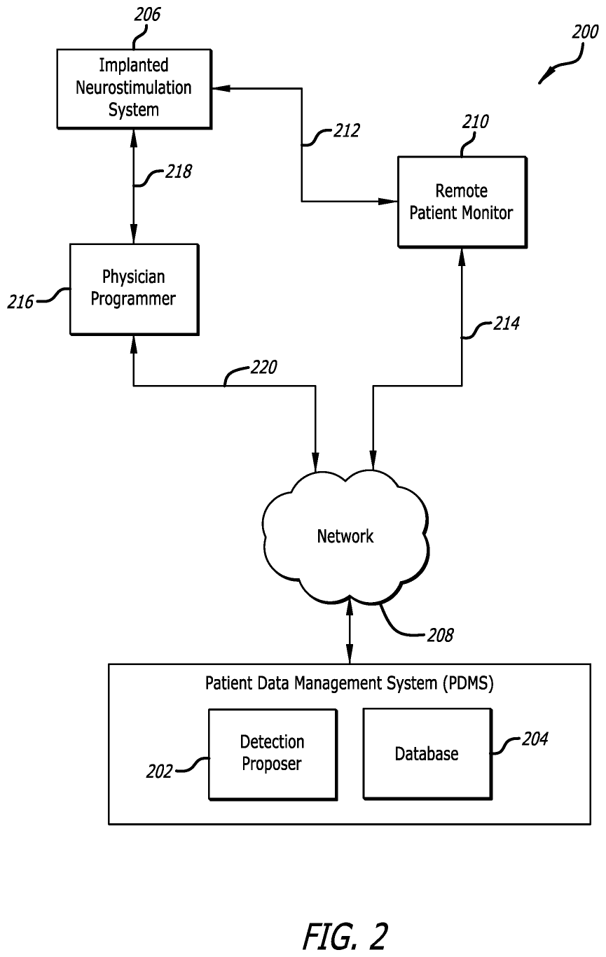

With reference to FIG. 2, a system 200 for processing physiological information, e.g., EEG records, to derive detection tools for a neurostimulation system 206 implanted in a patient includes a patient data management system (PDMS) 222, a physician programmer 216, and a remote patient monitor 210. The system 200 is configured to automatically determine and tune detection parameters for detection tools of the neurostimulation system 206. In an exemplary embodiment, detection settings are automatically determined and tuned (or adjusted) in the period of time between implant of a neurostimulation system 206 and when therapy is enabled in order to optimize a desired outcome measure.

The PDMS 222 includes a detection proposer 202 and a database 204. The detection proposer 202 is configured to analyze data and propose detection settings for detection tools, and to optimize performance of the detection tools prior to enablement of therapy. The database 204 stores datasets for processing by the detection proposer 202.

The remote patient monitor 210 interfaces with the neurostimulation system 206 to receive physiological information, e.g., EEG records, from the neurostimulation system 206. The remote patient monitor 210 also interfaces with the PDMS 222 through a network 208 to provide EEG records and other information to the database 204. The remote patient monitor 210 further interfaces with the PDMS 222 through the network 208 to obtain and automatically download to the neurostimulation system 206, the detection tools derived by the detection proposer 202.

The physician programmer 216 interfaces with the PDMS 222 through the network 208. The physician programmer 216 is configured to obtain information from the PDMS 222 related to the derivation and tuning of the detection tools, and to present the information on the user interface. For example, the information can include the candidate detection sets and proposed detection tools, as determined by the detection proposer 202, and performance metrics, e.g., detection rate, detection lag, etc., for the proposed detection tools when applied to a set of EEG records. The physician programmer 216 is also configured to receive detection criteria, e.g., desired detection rate, through a user interface and to provide the detection criteria to the detection proposer 202, which criteria is used by the detection proposer 202 to derive and tune detection tools for a neurostimulation system 206. The physician programmer 216 is also configured to obtain and automatically download to the neurostimulation system 206, the detection tools derived by the detection proposer.

### Implanted Neurostimulation System

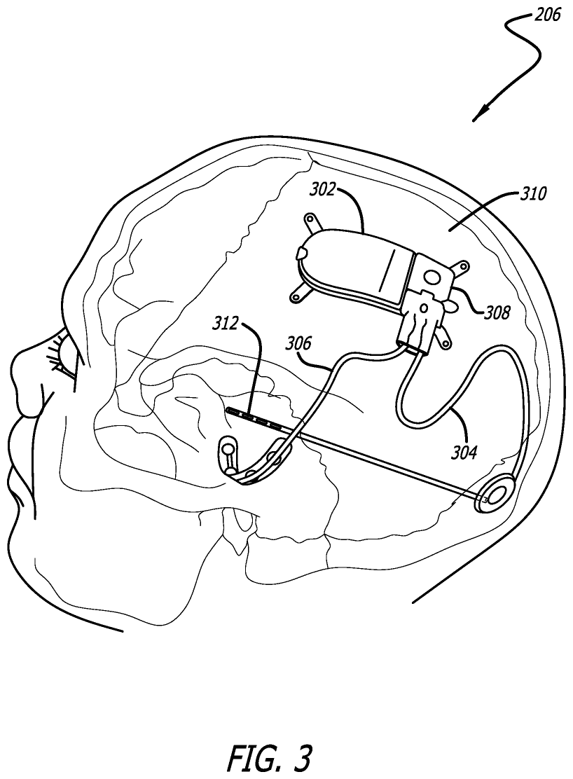

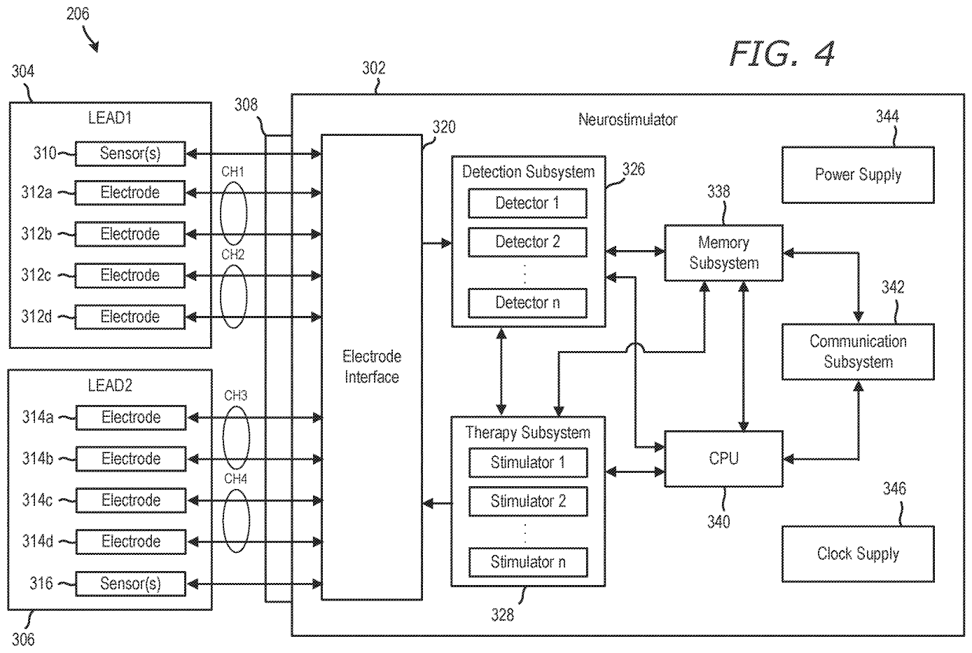

With reference to FIGS. 3 and 4, the neurostimulation system 206 includes a neurostimulator 302 and two electrode-bearing brain leads 304, 306. The neurostimulator 302 includes a lead connector 308 adapted to receive a connector end of the brain leads 304, 306, to electrically couple each lead and its associated electrodes 312a-d, 314a-d with the neurostimulator. The brain leads include a depth lead 304 and a cortical strip lead 306. The depth lead 304 is implanted so that a distal end of it is situated within the patient's neural tissue, whereas the cortical strip lead 306 is implanted under the dura mater so that a distal end of it rests on a surface of the brain. In some embodiments the brain leads may include leads whose distal form factor is furcated, gridded, helical, or planar. In some embodiments the brain leads may include leads whose distal end is situated in or substantially in a cerebral ventricle, a blood vessel, the epidural space, the subgaleal space, and/or the volume of the skull between inner table and outer table. In some embodiments, the neurostimulator 302 is positioned near the patient's neural tissue, for example intracalvarially or in a full- or partial-thickness craniotomy, and includes one or more conductive elements, such as an electrically active metallic outer casing, in lieu of one or more brain leads.

The neurostimulator 302 can configure one or more channels, each comprising a pair of the electrodes 312a-d, 314a-d, as either a sensing channel (for purposes of sensing electrical activity of the brain) or a stimulation channel (for purposes of delivering therapy to the patient in the form of electrical stimulation) or both. To these ends, the electrodes 312a-d, 314a-d are connected to an electrode interface 320 that is configured to form these channels. The electrode interface 320 also provides other features, capabilities, or aspects, including but not limited to amplification, isolation, and charge-balancing functions, that are required for a proper interface with neurological tissue.

With respect to sensing channels, the electrode interface 320 is configured to form a sensing channel by coupling a pair of electrodes to a detection subsystem 326 that includes one or more detectors configured to process electrical activity of the brain sensed through the pair of electrode 312a-d, 314a-d. Each detector is applied to a sensing channel, which is coupled to a pair of electrodes. In some cases, two or more detectors are applied in parallel to a sensing channel. In some embodiments, two or more detectors of the detection subsystem 326 are applied to the same sensing channel, where a first detector is coupled to the therapy subsystem 328 and a second detector is not coupled to the therapy subsystem 328. The first detector, also referred to as an active detector, governs delivery of stimulation therapy in response to its detections. The second detector, also referred to as a candidate detector, does not cause delivery of stimulation therapy in response to its detections; instead, the candidate detector's detections are used to log detection counts, to trigger storage of EEG records by the memory subsystem 338, and/or to provide data to the detection proposer 202 for the purposes of automatically determining or tuning detection parameters as disclosed herein. The configuration information of a candidate detector can subsequently be transferred to an active detector, or the candidate detector can be re-designated as an active detector, as disclosed further below. Each detector is defined by a set of detection parameters to detect a particular event, e.g., abnormal electrographic activity, an electrographic seizure, or the onset of an electrographic seizure, of a particular type, e.g., low voltage fast, hypersynchronous, etc., in a sensed EEG.

For example, a half wave detector can require specifying several parameters, including: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A line length detector can require a minimum of eight parameters to be specified, including: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

The neurostimulation system 206 is configured to generate records of electrical activity based on an occurrence of an event detected by a detection tool, or an occurrence of a trigger. To this end, the neurostimulation system 206 can be configured to create an EEG record of a sensed EEG when an event, e.g., an electrographic seizure or the onset of an electrographic seizure, is detected by a detection tool, and to create an EEG record of the corresponding EEG signal spanning the time period 60 seconds before the event was detected and 30 seconds thereafter. The neurostimulation system 206 can also be programmed to create an EEG record of a sensed EEG at certain times of day (e.g., at noon and at midnight). These are sometimes referred to as “scheduled EEGs.” In addition, the neurostimulation system 206 can be configured to store an EEG record upon some other trigger, such as when the patient swipes a magnet over the location on the patient's body at which the neurostimulator is implanted (the patient might be instructed to do this whenever he or she thinks a seizure is coming on).

In some embodiments, the neurostimulation system 206 is programmed to designate EEG records based on the event that triggered its recording and to include that designation in the EEG record. For example, EEG records resulting from the detection of abnormal electrical activity, e.g., an electrographic seizure or the onset of an electrographic seizure, are marked as such, while EEG records that do not reflect abnormal activity are designated as baseline EEG records. Thus, for a given patient, a dataset can contain EEG records corresponding to what is happening in the patient's brain during and around when an event occurs, scheduled EEG records acquired at a particular time, and EEG records stored by the neurostimulator when a patient triggers storage with a magnet. Some of these EEG records, especially the ones recorded at the time of an event or when triggered by a magnet swipe, may reflect the patient's electrographic seizures. The dataset can include information or a data type about whatever triggered the neurostimulator to store a given EEG, such as the type of event (e.g., Pattern “A” or Pattern “B,” a magnet swipe) or the time of day (e.g., scheduled EEG).

In some embodiments, the neurostimulation system 206 is configured to capture different data types based on EEG signals. Data types can be captured at different time scales. Some examples of data types captured by a neurostimulation system 206 include: (1) continuous recordings (EEG records) of raw brain data at a certain sampling rate such as 1000, 500 or 250 Hz, (2) continuous measures of derived brain data such as spectral power in certain frequency bands (example 1-4 Hz band, 4-8 Hz band, 8-12 Hz band, 12-25 Hz band, 25-50 Hz band, 50-90 Hz band and so on) computed in small moving and overlapping time windows such as 128, 256 or 512 milliseconds; (3) counts of abnormal events in bins of varying durations such as minutes, days or hours; (4) sampled raw time series or derived brain data that are saved at random time points, specific time points (preprogrammed by a physician for example) or are sampled in response to a trigger such as detection of abnormal events in brain or when a patient swipes a magnet over the neurostimulator; and (5) patient reports of outcomes. These are almost always not continuous and only intermittently available. In some embodiments, detection counts and EEG records collected by the neurostimulation system 206 are tagged with information identifying the detector instance whose detection caused the count to be incremented or the EEG record to be stored, e.g., an active detector or a candidate detector, so that data attributable to an active detector can be distinguished from data attributable to a candidate detector when uploaded to the PDMS 222 for processing by the detection proposer 202.

With respect to stimulation channels, the electrode interface 320 is configured to form one or more stimulation channels by coupling a pair of electrodes to a therapy subsystem 328, which is configured to deliver electrical stimulation therapy to the patient through the electrode 312a-d, 314a-d. The therapy subsystem 328 includes one or more stimulators, each defined by a set of stimulation parameters to deliver electrical stimulation therapy in response to “events” detected by the detection subsystem 326. The stimulation parameters can include: (1) a stimulation path that defines the electrodes through which stimulation is delivered, (2) a pulse width in microseconds, (3) a pulse frequency in Hz, (4) a pulse current in milliamps, (5) a burst duration in seconds, and (6) a pulse charge density, which is calculated from current, pulse width, and electrode surface area.

In some embodiments, one or both of the brain leads 304, 306 have one or more physiological sensors 310, 316 that enable the capture and recording of other types of physiological information, e.g., pH levels, blood oxygen levels, neurotransmitters concentrations, heart rate, blood pressure, blood glucose levels, hormone levels, sleep states, posture, etc. To this end, one or both of the brain leads 304, 306 is configured as disclosed in U.S. Pat. No. 10,123,717, entitled Multimodal Brain Sensing Lead, which is herein incorporated by reference, and the one or more physiological sensors 310, 316 correspond to different transducers, e.g., macroelectrodes, microelectrodes, light emitters, and photodetectors that enable different sensing modalities.

In some embodiments, the neurostimulation system 206 includes one or more electrodes configured to sense electrical cardiac activity indicative of heart rate, a pressure sensor configured to provide signals indicative of blood pressure, an accelerometer configured to provide motion signals indicative of motion and the position of the patient. From these accelerometer signals, the neurostimulation system 206 can derive other physiological information corresponding to clinical seizures, patient posture, and sleep state.

In some embodiments, other types of physiological information is obtained and stored by the neurostimulation system 206 from sources independent of the neurostimulation system. For example, an external wearable device, e.g., patch, can include a sensor configured to sense and track cortisol levels, i.e., stress hormones, in sweat, while an external wearable device, e.g., watch, can include or a sensor configured to sense blood pressure. The physiological information from these external devices is transmitted to the implanted neurostimulation system 206 for inclusion in the patient's dataset.

With reference to FIG. 4, the neurostimulator 302 includes a memory subsystem 338 and a central processing unit (CPU) 340, which can take the form of a microcontroller. The memory subsystem 338 is coupled to the detection subsystem 326, and receives and stores records of data representative of sensed electrographic signals for subsequent transmission to the database 204. The memory subsystem 338 is also coupled to the therapy subsystem 328 and the CPU 340.

The neurostimulator 302 also includes a communication subsystem 342. The communication subsystem 342 enables communication between the neurostimulator 302 and an external device, such as a physician programmer 216 or remote patient monitor 210, through a wireless communication link. The neurostimulator 302 also includes a power supply 344 and a clock supply 346. The power supply 344 supplies the voltages and currents necessary for each of the other subsystems. The clock supply 346 supplies substantially all the other subsystems with any clock and timing signals necessary for their operation.

The neurostimulator 302 is configured to be set to an adaptive mode (or adaptive phase) and a therapy mode (or therapy phase). During the adaptive phase, detection tools for the neurostimulator 302 are derived and optimized. During the therapy phase, the neurostimulator 302 detects events based on the optimal detection tool and delivers neurostimulation in response to such detections.

During a first stage of the adaptive phase, detection tools of the neurostimulator 302 are initially programmed with default detection parameters that detect for a pattern in electrographic activity of the brain. The neurostimulator 302 processes sensed EEG signals using the default detection tools and collects data, e.g., detection counts and EEG records, when the pattern of electrographic activity is detected. The data is uploaded to the PDMS 222.

After sufficient data is collected based on the default detection tools, the detection proposer 202 determines and proposes detection tools using this data as disclosed in detail later herein. Sufficient data is typically collected in 1-7 days. The proposed detection tools are automatically programmed into the neurostimulator 302. The neurostimulator 302 processes sensed EEG signals using the proposed detection tools and collects data, e.g., detection counts and EEG records, when the pattern of electrographic activity is detected. This data is uploaded to the PDMS 222. One or more parameters of the proposed detection tools are then coarsely adjusted using this data. Several iterations of coarse adjustments can occur.

During a second stage of the adaptive phase, one or more parameters of the coarse adjusted detection tools are further adjusted using data collected based on the coarse adjusted detection tools. Several iterations of incremental adjustments can occur. During this stage, one or more parameters of the proposed detection tools are adjusted (through a titration process) and performance of the detection tools are assessed as disclosed in detail later herein. The adjusted or tuned detection tools are automatically programmed into the neurostimulator 302. The neurostimulator 302 processes sensed EEG signals using these tuned detection tools, collects data, e.g., detection counts and EEG records, when the pattern of electrographic activity is detected, and uploads the EEG records to the PDMS 222. The titration process is periodically repeated during the second stage to determine an optimal detection tool. Next, the optimal detection tool is programmed into the neurostimulation system 206 and the neurostimulator 302 is set to therapy mode. The programming of the optimal detection tool may be initiated by a physician or it may be automatic. Likewise, the setting of the neurostimulator 302 to the therapy mode may be initiated by a physician or it may be automatic.

In some embodiments, the adaptive phase described above is performed prior to enabling delivery of stimulation therapy by the neurostimulator 302, as disclosed in detail later herein. In other embodiments, the adaptive phase, or any one or more stages of the adaptive phase, is performed concurrently with delivery of stimulation therapy by the neurostimulator 302. In such embodiments, the neurostimulator 302 includes one or more active detectors of the detection subsystem 326 whose detections govern delivery of stimulation therapy by the therapy subsystem 328, and one or more candidate detectors of the detection subsystem 326 whose detections do not govern delivery of stimulation therapy. The detection proposer 202 automatically determines and tunes detection parameters of the candidate detectors using the methods disclosed herein, while the neurostimulator 302 continues to deliver stimulation therapy under the control of the active detectors. After the detection parameters of a candidate detector have been determined and tuned to satisfy a target performance metric, the configuration information of the candidate detector is transferred to an active detector, or the candidate detector is re-designated as an active detector, such that subsequent stimulation therapy is delivered in response to detections by the tuned detector. Because a candidate detector is architecturally separated from the therapy subsystem 328 in that detections by the candidate detector do not cause delivery of stimulation therapy, the determination and tuning of detection parameters for the candidate detector does not modify the patient's ongoing stimulation therapy.

In some embodiments, the determination and tuning of one or more detection tools is performed in a single continuous operating period without distinct first and second stages and without distinct first and second periods of time. To this end, the detection proposer 202 interleaves operations of proposing a detection tool, coarsely adjusting one or more detection parameters of the detection tool, and titrating one or more detection parameters of the detection tool, in any order and any combination, in response to data received from the IMD 206. For example, in some embodiments the detection proposer 202 performs an initial proposal of a detection tool, programs the detection tool into the IMD 206, and thereafter continuously or periodically adjusts the detection tool's parameters as new data are received, without any externally defined transition from a coarse adjustment regime to a titration regime. In other embodiments, the detection proposer 202 performs only one of coarse adjustment or titration, with the other omitted. In other embodiments, only a determination of the detection tool is performed and no subsequent adjustment is performed. In each of the foregoing embodiments, the operations attributed elsewhere in this disclosure to the first stage and the second stage are not limited to a particular temporal order or partition.

### Detection Proposer

The detection proposer disclosed herein is configured to: 1) classify EEG records sensed by an implanted medical device into seizure EEG records and non-seizure EEG records, 2) to group and process seizure EEG records based on seizure onset times and electrographic activity types to determine candidate sets of detection parameters, also referred to as “candidate detection sets,” 3) to build and test detection tools based on the candidate detection sets, and 4) to select one or more detection tools for programming into the implanted medical device based on the outcome of the tests.

Example detection tools include the above-described half wave detector, line length detector and an area detector. As previously mentioned, a detection set, also referred to as a parameter set, for a half wave tool can include: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A detection set for a line length tool can include: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

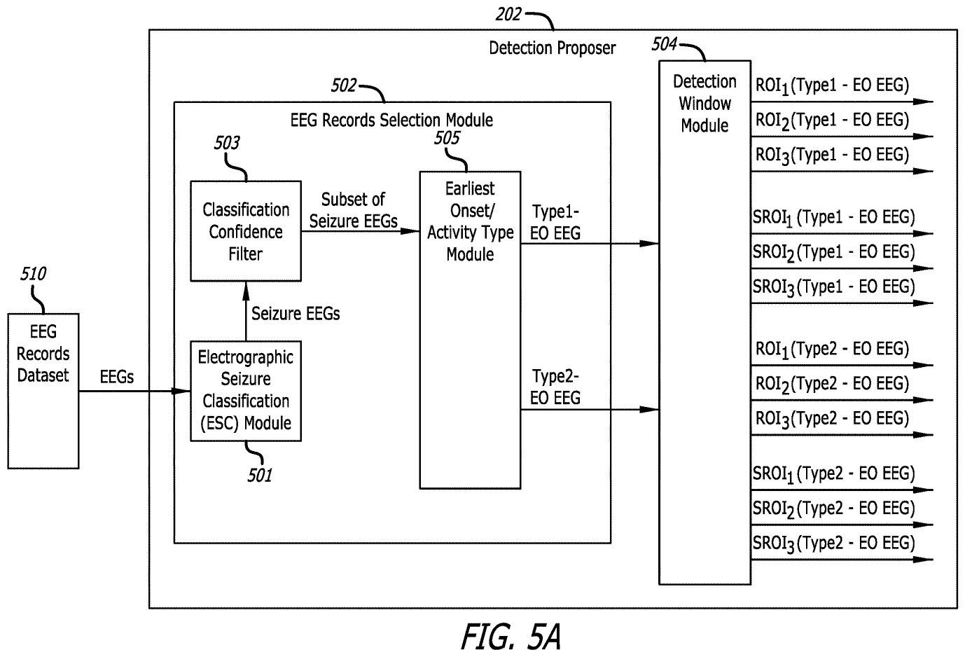

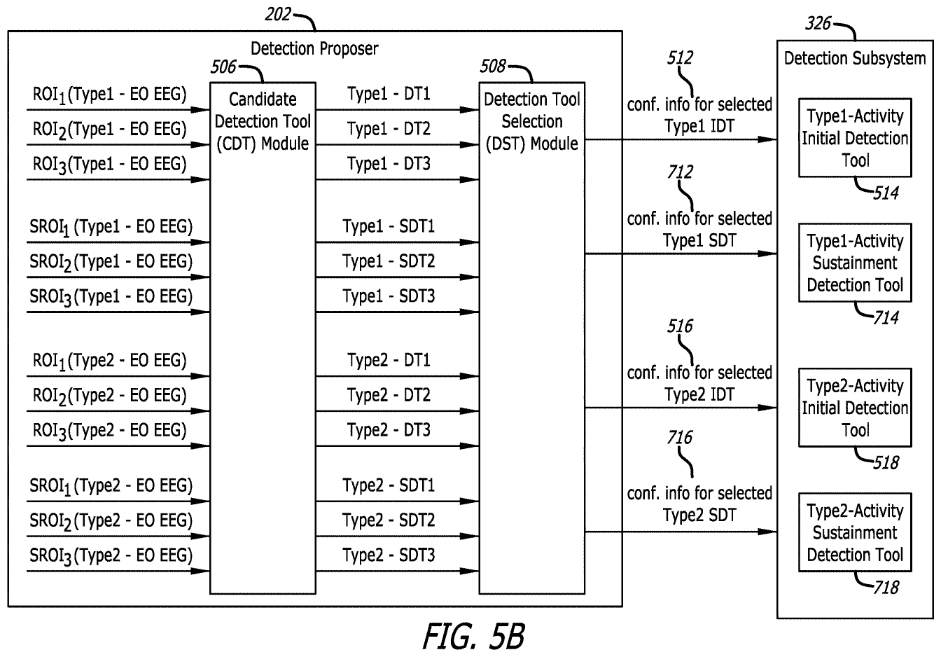

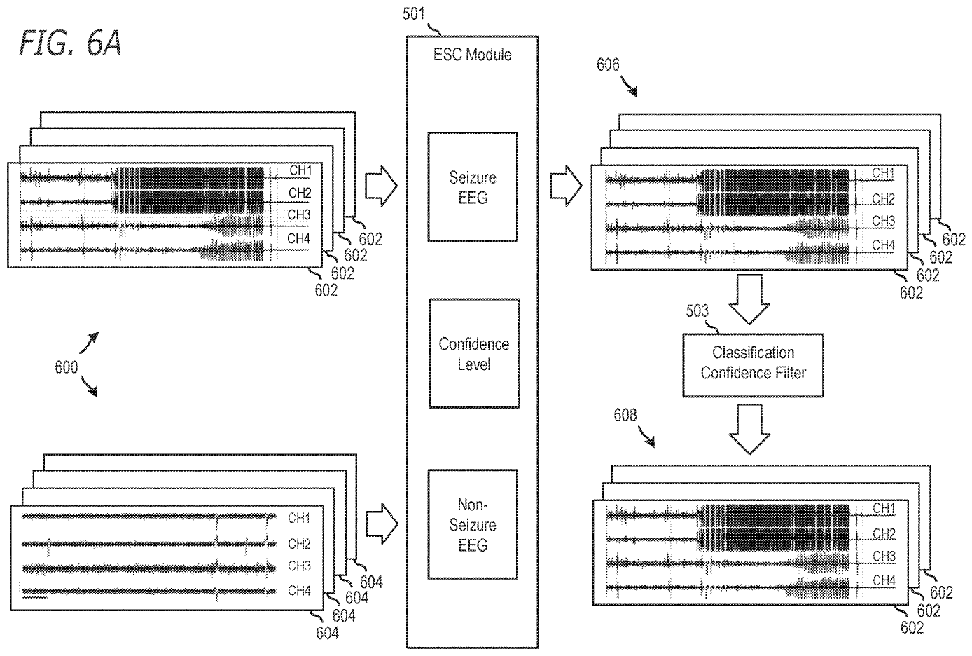

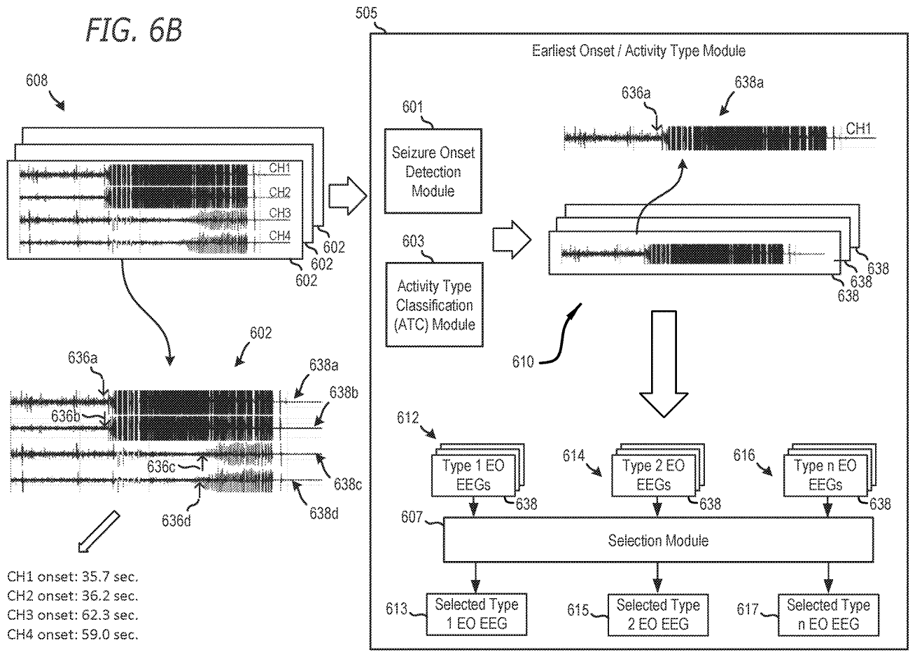

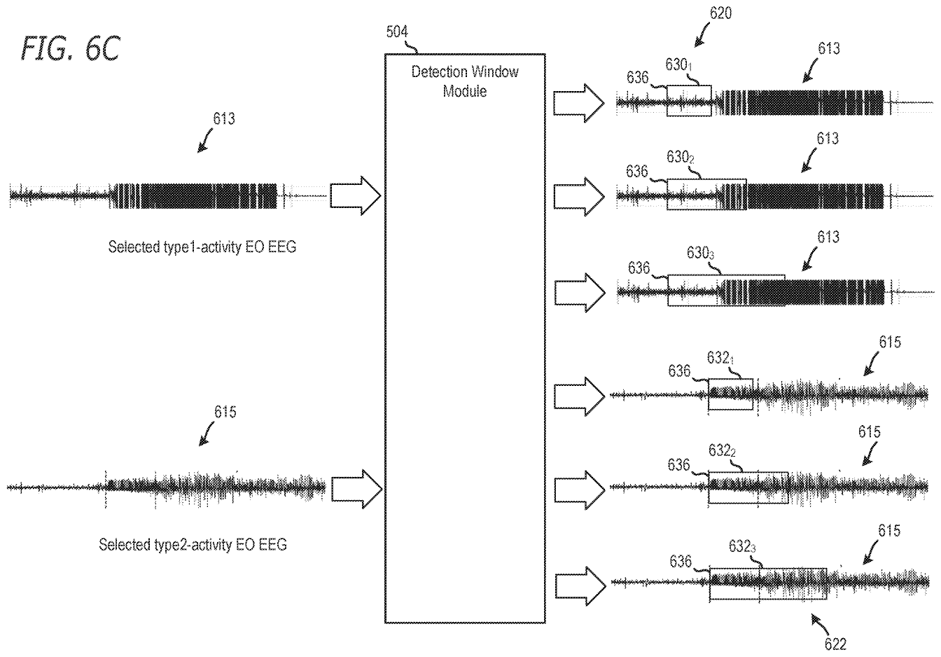

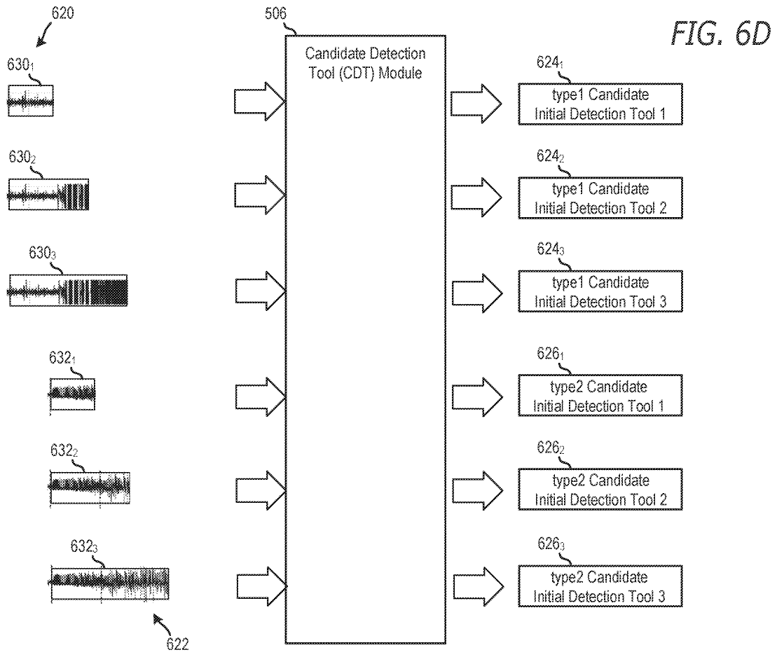

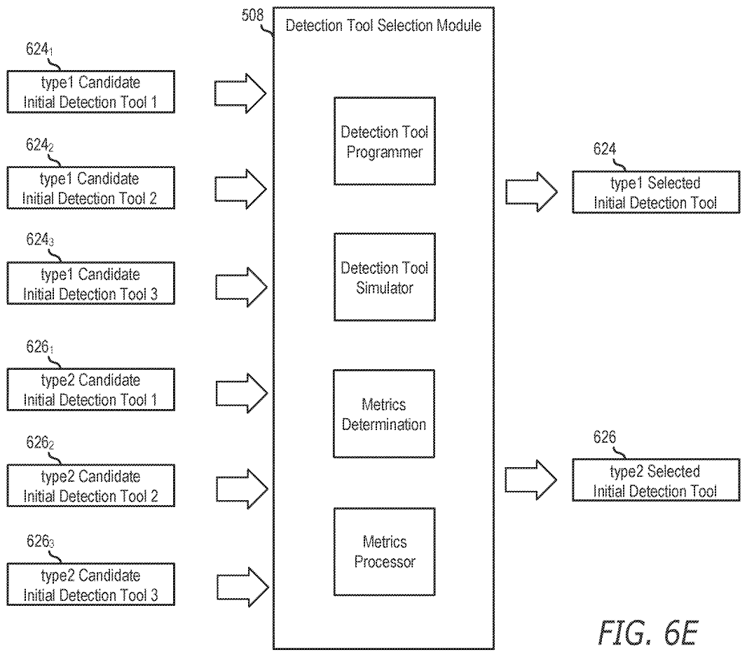

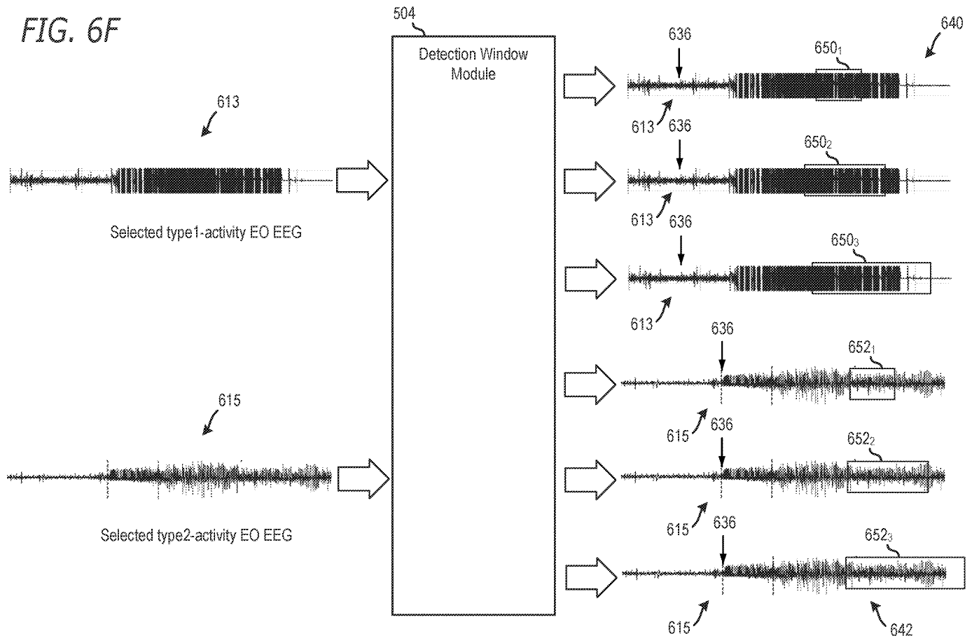

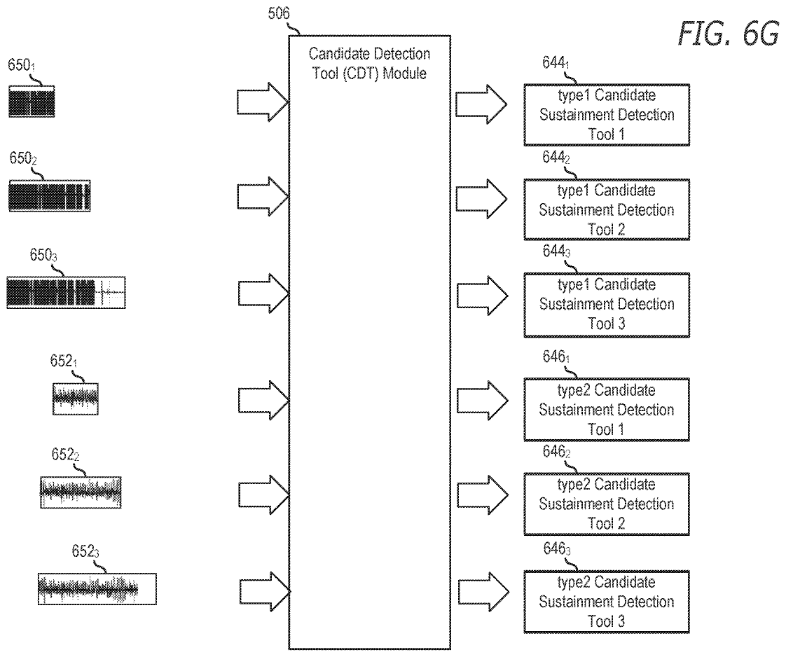

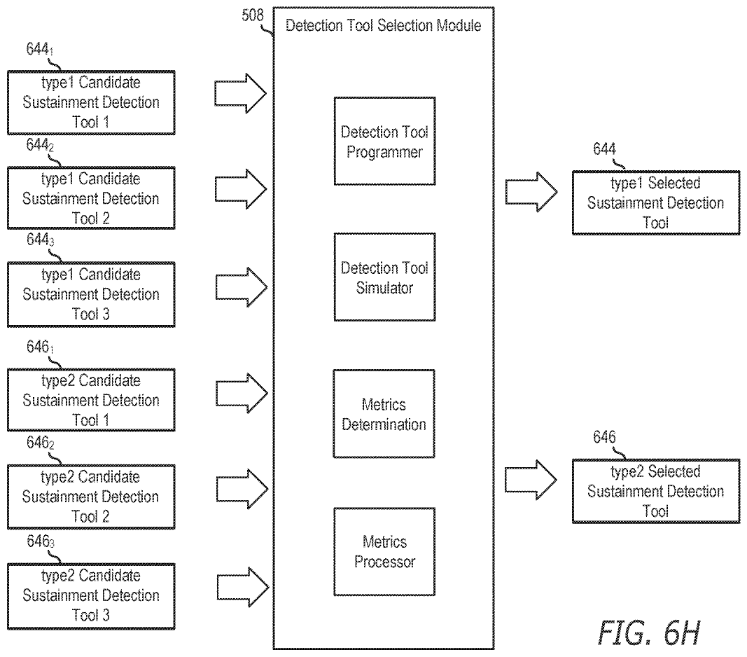

With reference to FIGS. 5A and 5B, in an example configuration the detection proposer 202 includes an EEG records selection module 502, a detection window module 504, a candidate detection tool (CDT) module 506, and a detection tool selection (DST) module 508. The EEG records selection module 502 includes an electrographic seizure classification (ESC) module 501 (also referred to as an ESC model), a classification confidence filter 503, and an earliest-onset/activity-type (EO/AT) module 505 (also referred to as an EO/AT model). Additional modules and functionalities of the EEG records selection module 502, the detection window module 504, the CDT module 506, and the DST module 508 are disclosed below with reference to FIGS. 6A-6E.

As shown in FIG. 5A, the detection proposer 202 is configured to interface with an EEG records dataset 510, which can be part of the database 204 of FIG. 2, for purposes of receiving EEG records for processing. The detection proposer 202 also interfaces with a display (not shown) to enable the display of EEG records, the display of configuration information (e.g., detection tool type, detections parameter set) for candidate detection tools and selected detection tools, as determined by the detection proposer, and the display of metrics of detection tool performance, such as detection rate, detection lag, and false positive detection rate. The detection proposer 202 can also interface with a user interface (not shown) to receive inputs from a user that indicate a desired value for a metric. For example, a user can input a desired detection rate for a detection tool. Based on this input, the detection proposer 202 can select, from among a number of candidate detection tools, the tool having a detection rate that matches (or is within a tolerance of matching) the desired detection rate.

With reference to FIGS. 5A and 6A, in an operation of the detection proposer 202, the ESC model 501 of the EEG records selection module 502 is applied to a set 600 of multichannel EEG records (in this case the EEG records includes four channels: CH1, CH2, CH3, and CH4). The ESC model 501 is configured to classify each multichannel EEG record in the set 600 of records as either a seizure record 602 or a non-seizure record 604. The ESC model 501 is also configured to assign a confidence level that represents the likelihood that a multichannel EEG record 602 classified as a seizure record is indeed a seizure record. In one example, the ESC model 501 assigns a confidence level in the range of 0.1 to 0.9, where 0.1 indicates a low confidence level and 0.9 indicates a high confidence level.

In some embodiments, the ESC model 501 is a machine learning based model trained in accordance with one of the models or a combination of models described in Barry W, Arcot Desai S, Tcheng TK and Morrell MJ (2021), A High Accuracy Electrographic Seizure Classifier Trained Using Semi-Supervised Labeling Applied to a Large Spectrogram Dataset. Front. Neurosci. June 2021, Vol. 15, Art. 667373 (hereinafter referred to as Barry et al.), and/or Arcot Desai S, Afzal MF, Barry W, Kuo J, Benard S, Traner C, Tcheng T, Seale C and Morrell M (2023), Expert and deep learning model identification of iEEG seizures and seizure onset times. Front. Neurosci. July 2023, 17:1156838 (hereinafter referred to as Arcot Desai et al.), each of which is incorporated by reference. Also see Leveraging Artificial Intelligence Models for Classifying Electrographic Seizures and Identifying Seizure Onset Times in Patients Treated with the RNS System, AES abstract no. 2.186, published Dec. 8, 2024.

For example, a large dataset of EEG records from over a hundred patients can be manually labeled as seizures and non-seizures and used to train an attention-based ESC model 501 based on a convolutional neural network (CNN). One such trained model described in Barry et al. had a test classification accuracy of >95% on individual EEG channels which means the trained model could classify EEG channels from new patients as seizures and non-seizures with over 95% classification accuracy. In some embodiments, the attention-based ESC model 501 is based on a vision transformer architecture (ViT) or other neural network architecture.

With continued reference to FIG. 6A, a set 606 of multichannel seizure records 602 is applied to the classification confidence filter 503. The classification confidence filter 503 is configured to filter the set 606 of multichannel seizure records 602 based on a threshold confidence level to provide a subset 608 of multichannel seizure records 602 for further processing by the EO/AT module 505. In some embodiments, the classification confidence filter 503 is programmed to include multichannel seizure records 602 with a confidence level at or above the threshold confidence level in the subset 608 of seizure records. The threshold confidence level can be, for example, 0.8.

With reference to FIGS. 5A and 6B, a seizure onset detection (SOD) module 601 (also referred to as an SOD model) of the EO/AT module 505 is applied to the subset 608 of multichannel EEG records 602. The SOD module 601 is configured to predict a seizure onset 636a-d for each channel EEG record 638a-d in a multichannel seizure record 602 and to provide a corresponding seizure onset time. For example, in FIG. 6B, the CH1 EEG record 638a is determined to have a seizure onset 636a at a time of 35.7 seconds, the CH2 EEG record 638b is determined to have a seizure onset 636b at a time of 36.2 seconds, the CH3 EEG record 638c is determined to have a seizure onset 636c at a time of 62.3 seconds, and the CH4 EEG record 638d is determined to have a seizure onset 636d at a time of 59.0 seconds.

In some embodiments, the SOD module 601 comprises a convolutional neural network (CNN) based architecture. In this architecture, a large dataset of EEG records is manually labeled for seizure onset times on individual EEG channels and a CNN is trained on the labeled dataset. See, for example, Arcot Desai et al. The trained CNN is applied to a multichannel seizure record 602 to predict seizure onsets and provide times of seizure onsets for each channel in the record. In other embodiments, the SOD module 601 is a deep learning model created using different methods, such as attention models (vision transformers) or signal processing algorithms. See, for example, “Recurrent Neural Networks for Forecasting Epileptiform Electrographic Activity 24 Hours in Advance,” AES abstract no. 2.050, published Nov. 6, 2018. Also see Leveraging Artificial Intelligence Models for Classifying Electrographic Seizures and Identifying Seizure Onset Times in Patients Treated with the RNS System, AES abstract no. 2.186, published Dec. 8, 2024.

In some embodiments, the EO/AT module 505 includes an activity type classification (ATC) module 603 (also referred to as an ATC model) configured to classify seizure EEG records as a type of seizure onset. In one configuration, the ATC module 603 invokes a clustering method in which features are extracted from the EEG records and passed through a clustering algorithm such as Bayesian Gaussian Mixture Models as disclosed in Barry et al. to determine which type/pattern (or cluster) of seizure onset the EEG records falls within. The ATC module 603 is trained to recognize various type/patterns of electrical activity in EEG records corresponding to seizure onsets. Some examples are shown in Table 3 below along with a description. EEG examples with these activity patterns are further described in Nune et al., “Treatment of drug-resistant epilepsy in patients with periventricular nodular heterotopia using RNS System: Efficacy and description of chronic electrophysiological recordings,” Clinical Neurophysiology 2019, which is incorporated by reference.

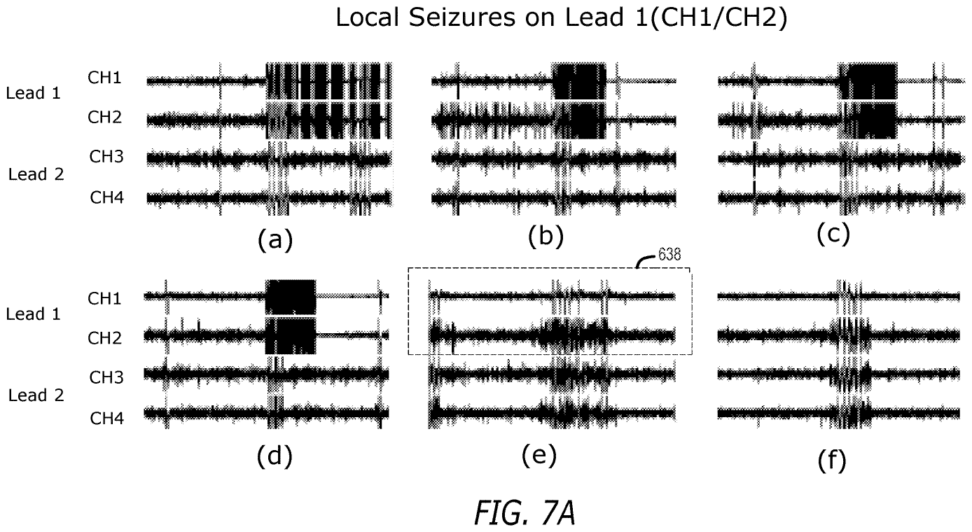

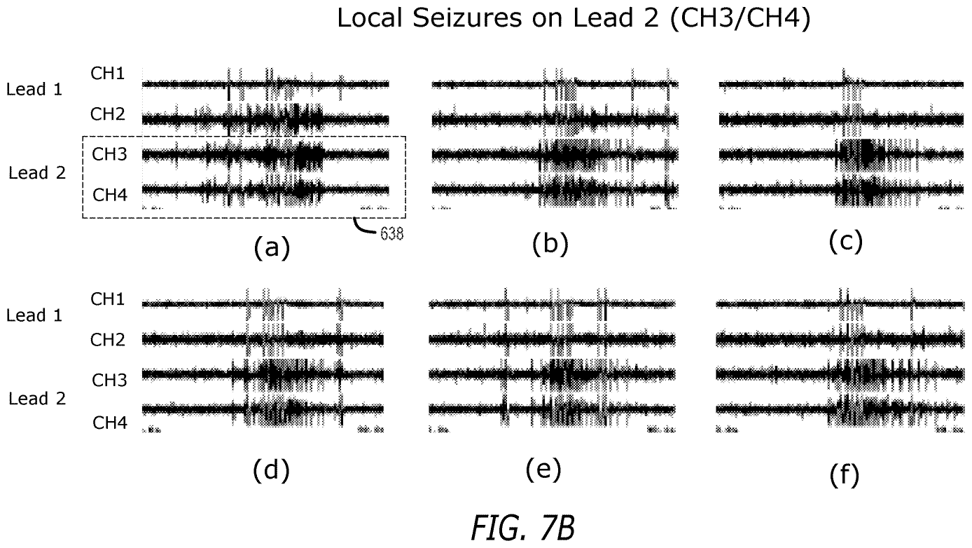

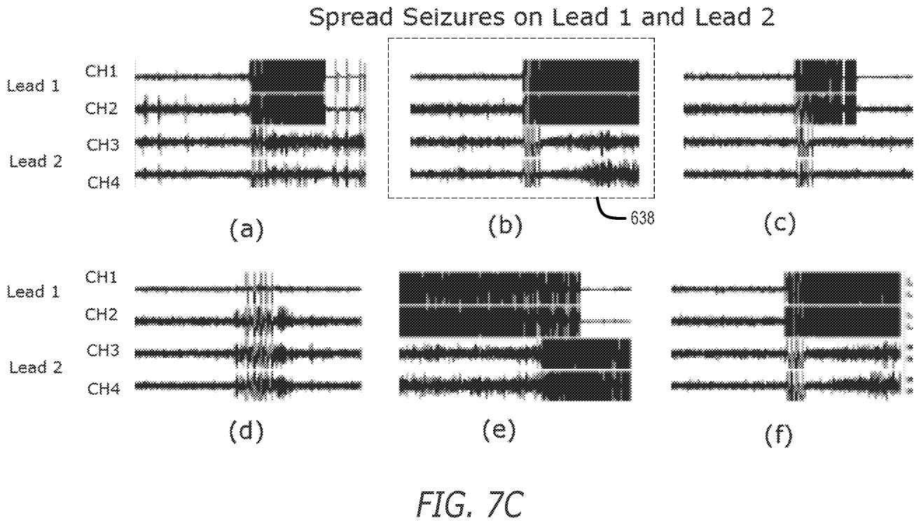

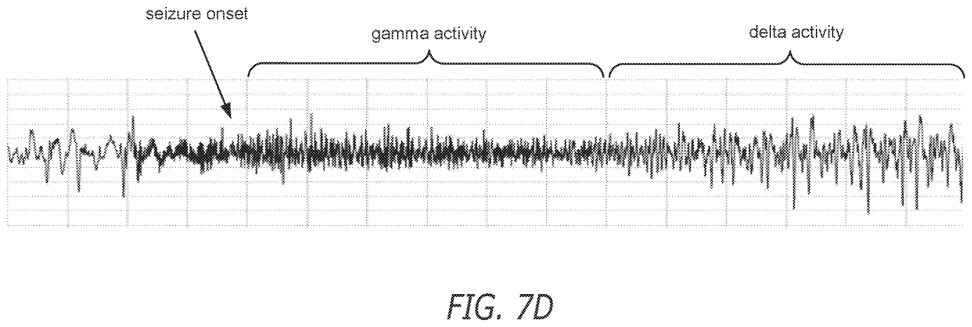

TABLE 3

| Seizure Onset Type | Description |
|---|---|
| Low Voltage Fast | Slowly evolving, high frequency (>13 Hz) and low |
| (LVF) | amplitude activity progressing to lower frequencies |
| | and higher amplitude. |
| Hypersynchronous | Periodic spiking at less than 3 Hz for a duration of |
| (Hypersync) | >10 seconds prior to seizure onset. |
| Attenuation (Atten) | Voltage suppression without a significant increase |
| | in power at any frequency. |
| Multiple (M) | Multiple concurrent onset types. |
| Rhythmic Delta (D) | Rhythmic activity at 1Hz to <4 Hz or periodic |
| | spiking at >3 Hz and <4 Hz. |
| Rhythmic Theta (T) | Rhythmic 4Hz to <8 Hz activity. E.g., see FIG. 7C, |
| | panel (b), lead 1 (CH1/CH2) and lead 2 (CH3/CH4). |
| Rhythmic Alpha (A) | Rhythmic 8Hz to <13 Hz activity |
| Semi-Rhythmic | High but variable amplitude, semi-rhythmic spiking |
| Beta (SRB) | in the beta frequency range. E.g., see FIG. 7A, |
| | panel (e), lead 1 (CH1/CH2) EEG record. |
| High Amplitude | High amplitude, semi-rhythmic spiking in the beta |
| Beta | frequency range. E.g., see FIG. 7B, panel (a), lead 2 |
| | (CH3/CH4) EEG record. |
| Transition Seizure | Initial activity, e.g., gamma activity, |
| | that transitions to a different activity, |
| | e.g., delta activity. E.g., see FIG. 7D |

With reference to FIG. 7A, the ATC module 603 can classify an EEG record of a seizure on lead 1 (CH1/CH2) as containing a first type of activity (type1-activity), such as semi-rhythmic beta type activity. An example of such an EEG record is shown in the lead 1 (CH1/CH2) EEG record, panel (e) of FIG. 7A. With reference to FIG. 7B, the ATC module 603 can classify an EEG record of a seizure on lead 2 (CH3/CH4) as containing a second type of activity (type2-activity), such as high amplitude beta activity. An example of such an EEG record is shown in the lead 2 (CH3/CH4) EEG record, panel (a) of FIG. 7B. With reference to FIG. 7C, the ATC module 603 can classify an EEG record of a seizure on lead 1 (CH1/CH2) and lead 2 (CH3/CH4) as containing an activity type 3, such as rhythmic theta. An example of such an EEG record is shown in panel (b) of FIG. 7C.

In some embodiments, the ATC module 603 is further configured to classify an EEG record as having a transition in activity type. For example, with reference to FIG. 7D, an electrographic seizure can start with primary gamma activity and transition to delta activity during the body of the seizure. In some embodiments, the ATC module 603 includes a submodule that determines an EEG record has a transition in activity type based on power spectral density (PSD) analysis. To this end, the ATC module 603 submodule computes PSDs for a moving window of the EEG record after the initial seizure onset to identify frequency peaks after seizure onset. If there are gamma peaks in a computed PSD, the portion of the EEG record within the window is still in the initial onset phase. An absence of gamma peaks along with a presence of only low frequency (e.g., delta) peaks in a computed PSD indicates the portion of the EEG record within the window is after the onset phase and includes the body of the seizure. An absence of peaks in the computed PSD indicates the portion of the EEG record within the window includes the end of the seizure (not shown in FIG. 7D).

In some embodiments, the ATC module 603 is further configured to classify an EEG record as a particular activity type based on the spread time (i.e., the difference in seizure onset time on CH1/CH2 vs CH3/CH4) for that EEG record. For example, an EEG record with a spread time above a threshold value, e.g., 10 seconds, and brain activity frequency above a certain threshold, e.g., 50 Hz, is classified by the ATC module 603 as a low voltage fast activity pattern. As another example, if the spread time is below 5 seconds and the brain activity frequency is below 20 Hz, the ATC module 603 can classify it as theta onset pattern.

As an alternative to a clustering algorithm, the ATC module 603 can invoke a rule based method in which features extracted from the EEG records are used to classify a seizure based on pre-set rules. Such rule based methods are disclosed in U.S. Pat. No. 10,543,368, “Seizure Onset Classification and Stimulation Parameter Selection,” which is incorporated by reference. Examples of features extracted from EEG records include spectral power in delta, theta, alpha, beta, low gamma and high gamma bands. A list of other features is disclosed in U.S. Pat. No. 10,543,368.

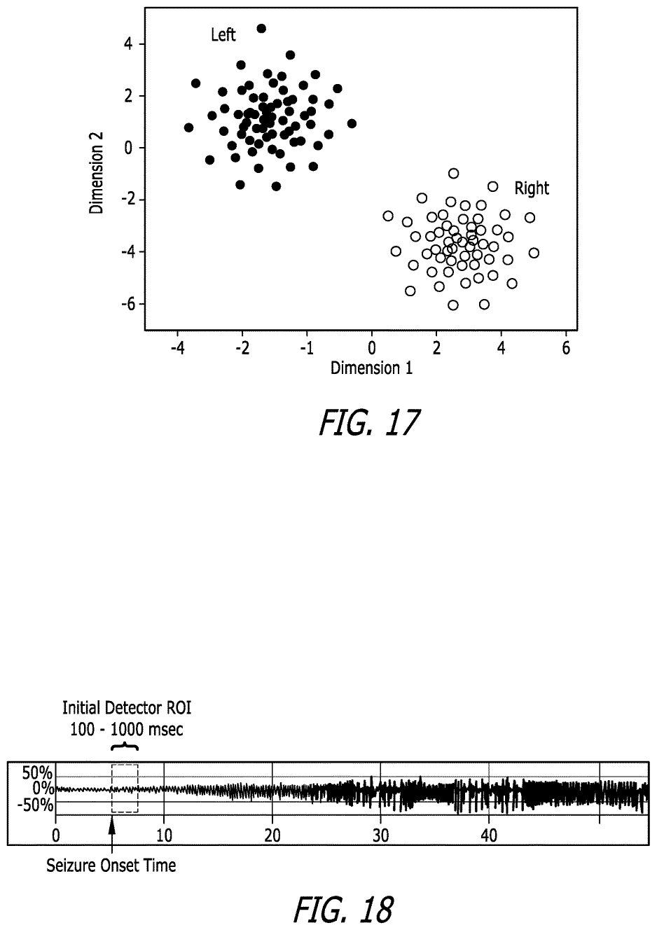

Continuing with reference to FIGS. 5A and 6B, the ATC module 603 provides a set 610 of earliest onset (EO) EEG records 638. Note, for ease of illustration the EO EEG records 638 shown in FIG. 6B are single-channel, e.g., CH1, CH2, CH3, CH4, etc. EEG records. In practice, the EO EEG records 638 can include multiple channels. For example, with reference to FIGS. 7A and 7B, the EO EEG records 638 can include two channel records, e.g., Lead 1 (CH1/CH2), Lead 2 (CH3/CH4), and can be clustered by lead and seizure onset type. An example of such a cluster is shown in FIG. 17. With reference to FIG. 7C, in the case of spread seizures the EO EEG records 638 can include four channel records, e.g., Lead 1 (CH1/CH2) and Lead 2 (CH3/CH4), and can be clustered together as a spread seizure.

In the example of FIG. 6B, the set 610 of EO EEG records 638 are grouped into “n” different subgroups based on seizure onset types and channel. The subgroups can include a set 612 of type1-activity earliest onset (EO) EEGs on CH1, a set 614 of type2-activity EO EEGs on CH4, through to a set 616 of typen-activity EO EEGs on a channel. In an example, the type1-activity onset is a low voltage fast (LVF) seizure onset, while the type2-activity onset is a hypersynchronous seizure onset. In this instance, each set 612, 614, 616 is processed as described later below to propose a detection tool for its respective channel.

In another example, the EO EEG records 638 are multi-channel EEG records and can be grouped into “n” different subgroups based on seizure onset types and lead 1 (CH1/CH2) or lead 2 (CH3/CH4). The subgroups can include a set 612 of type1-activity earliest onset (EO) EEGs on lead 1 (CH1/CH2), a set 614 of type2-activity EO EEGs on lead 2 (CH3/CH4), through to a set 616 of typen-activity EO EEGs on a lead. In this instance, each set 612, 614, 616 is processed as described later below to first determine a detection channel from among its respective channels, e.g., lead 1 (CH1/CH2) or lead 2 (CH3/CH4), and then to propose a detection tool for its respective determined detection channel.

Continuing with the example of FIG. 6B, the set 612 of type1-activity EO EEGs, the set 614 of type2-activity EO EEGs through to the set 616 of typen-activity EO EEGs are applied to a selection module 607 of the EO/AT module 505. In some embodiments, the selection module 607 is configured to separately process each set 612, 614, 616 of earliest onset EEG records to identify, for each set, the earliest onset EEG record 613, 615, 617 with the earliest seizure onset time. Because the sets 612, 614, 616 of earliest onset EEGs records are grouped by seizure onset type, the selection module 607 in effect, identifies for each seizure onset type, the earliest onset EEG record 613, 615, 617 with the earliest seizure onset time.

In instances where the EO EEG records 638 are multi-channel records, e.g., type1-activity earliest onset (EO) EEGs on lead 1 (CH1/CH2) or type2-activity EO EEGs on lead 2 (CH3/CH4), the selection module 607 is configured to determine a detection channel while identifying the earliest onset EEG record 613, 615, 617 with the earliest seizure onset time. In some embodiments, the selection module 607 calculates an overall onset time based on the individual onset times of the EO EEG records 638 for each channel in a multi-channel EEG record. The overall onset time can be the mean of the individual onset times. Alternatively, the overall onset time can be the median of the individual onset times. The selection module 607 selects the channel having the earliest overall onset time as the detection channel. The selection module 607 compares the individual onset times of the EEG records of the detection channel to determine the earlier onset time and identifies the individual EEG record associated with the determined earliest onset time as the earliest onset EEG record 613, 615, 617 with the earliest seizure onset time.

In alternative embodiments, the selection module 607 is configured to separately process each set 612, 614, 616 of earliest onset EEG records to identify, for each set, an EEG record 613, 615, 617 that is characteristic of that set in place of or in addition to having the earliest seizure onset time. For example, this characteristic may be a record whose location in a parameter space used for clustering by the ATC module 603 is nearest to the centroid of the cluster to which that record belongs. An example of such clustering is shown in FIG. 17. As another example, this characteristic can be a record whose likelihood of containing a certain activity type as determined by the ATC module 603 is higher than that of the other records in the set. As another example, this characteristic can be the first record available in the set. As yet another example, this characteristic can be the record having less artifact than the other records in the set.

With reference to FIGS. 5A and 6C, the detection window module 504 receives and separately processes the earliest onset EEG records 613, 615. For clarity of illustration, the earliest onset EEG record 617 is not included in FIG. 6C. The detection window module 504 is configured to identify, for the type1-activity earliest onset EEG record 613, a first set 620 of regions of interest (ROIs) 630₁, 630₂, 630₃, each having a different length within a range of lengths, and to identify, for the type2-activity earliest onset EEG record 615, a second set 622 of ROIs 632₁, 632₂, 632₃, each having a different length within a range of lengths. These ROIs may also be referred to as detection windows.

In some embodiments, the detection window module 504 is alternatively or additionally configured to identify sets of ROIs taken from multiple records. For example, the detection window module 504 may identify a first set 620 of ROIs where a single ROI, or a subset of ROIs having at least a first length and a second length, is drawn from each record in set 612 of type1-activity earliest onset (EO) EEGs. In this example, the detection window module 504 may also identify a second set 622 of ROIs where a single ROI, or a subset of ROIs having at least a first length and a second length, is drawn from each record in set 614 of type2-activity earliest onset (EO) EEGs.

In some embodiments, each ROI in a set of ROIs begins at the time of the seizure onset 636 within the earliest onset EEG record 613, 615. In some embodiments, the respective durations of ROI in a set of ROIs are based on programmed settings. For example, the duration or period of time of the first ROI 630₁, is d, the duration of the second ROI 630₂, is a×d, and the duration of the third ROI 630₃, is b×d, where variables a, b and d are predetermined. In some embodiments, the range of lengths is based on the seizure onset type of the earliest onset EEG record. Example ranges for ROI lengths are provided in Table 4 below:

TABLE 4

| Seizure Onset Type | ROI length range (in seconds) |
|---|---|
| Low Voltage Fast (LVF) | 1-2 |
| Hypersynchronous (Hypersync) | 2-4 |
| Attenuation (Atten) | 1-2 |
| Multiple (M) | 2-3 |
| Rhythmic Delta (D) | 2-4 |
| Rhythmic Theta (T) | 2-3 |
| Rhythmic Alpha (A) | 1.5-2.5 |
| Semi-Rhythmic Beta (SRB) | 1.5-2.5 |

In some embodiments disclosed further below with reference to FIGS. 6F, 6G, and 6H, an additional ROI is identified for each earliest onset EEG record 613, 615 and processed to propose an additional detector for a sensing channel. This additional detector is referred to as a “sustainment detector.” The purpose of the sustainment detector is to detect for a type of electrographic activity that typically follows the type of activity intended to be detected by an initial detector coupled to the sensing channel. In other words, the sustainment detectors are intended to detect evolved seizure body patterns rather than onset morphology, which is detected by initial detectors.

With reference to FIGS. 5B and 6D, the candidate detection tool (CDT) module 506 is configured to determine a corresponding type1 candidate initial detection tool 624₁, 624₂, 624₃ based on each ROI 630₁, 630₂, 630₃ and a corresponding type2 candidate initial detection tool 626₁, 626₂, 626₃. based on each ROI 632₁, 632₂, 632₃. In some embodiments, the CDT module 506 is alternatively or additionally configured to determine a set of type1 candidate initial detection tools 624₁, 624₂, 624₃ based on ROIs taken from two or more records from set 612 of type1-activity earliest onset (EO) EEGs, and a corresponding set of type2 candidate initial detection tools 626₁, 626₂, 626₃ based on ROIs from two or more records from set 614 of type2-activity earliest onset (EO) EEGs. In some embodiments, the CDT module 506 is configured to determine one set of candidate initial detection tools 624₁, 624₂, 624₃ based on each ROI 630₁, 630₂, 630₃ for only one activity type; in some embodiments, the CDT module 506 is configured to determine more than two sets of candidate detection tools based on more than two activity types.

As an initial step in determining candidate initial detection tools, the CDT module 506 preprocesses each ROI to remove artifacts. The ROIs may have flat artifacts due to the brain implanted electrodes having dual tasks of sensing the EEG signal and delivering stimulation. Therefore, during those brief periods (hundreds of milliseconds) when the electrodes are not sensing the EEG signal, the neurostimulator records a flat artifact. In some embodiments, the CDT module 506 processes the ROI to remove these artifacts. In some embodiments, stimulation artifacts are removed by calculating when stimulation was delivered using EEG metadata, then removing the artifacts. In further embodiments, the artifact portions of the EEG signals that are removed are replaced with reconstructed brain activity. For example, machine learning models can be used to model and reconstruct brain activity data obscured by stimulation artifacts. See Menon K, Tcheng T, Seale C, Greene D, Morrell M and Desai SA (2025) Reconstructing signal during brain stimulation with Stim-BERT: a self-supervised learning model trained on millions of iEEG files. Front. Artif. Intell. 8:1502504. doi: 10.3389/frai.2025.1502504. This method enables the estimation and replacement of missing or corrupted time series segments with learned representations of brain activity, thereby preserving the integrity of the underlying brain signals. In some embodiments, the artifact removal or reconstruction methods described here may be applied prior to use of the CDT module 506, for example before records are presented to the ESC module 501, before records are presented to the EO/AT model 505, or before records are presented to the detection window module 504.

After preprocessing of the ROIs, each type1 ROI 630₁, 630₂, 630₃ is individually processed by the CDT module 506 to determine a corresponding activity-type 1 candidate initial detection tool 624₁, 624₂, 624₃. Likewise, each type2-activity ROI 632₁, 632₂, 632₃ is individually processed by the CDT module 506 to determine a corresponding type2 candidate initial detection tool 626₁, 626₂, 626₃. The respective detection tools are specified by configuration information that includes detection-tool type, e.g., halfwave, line length, etc., and a detection parameter set.

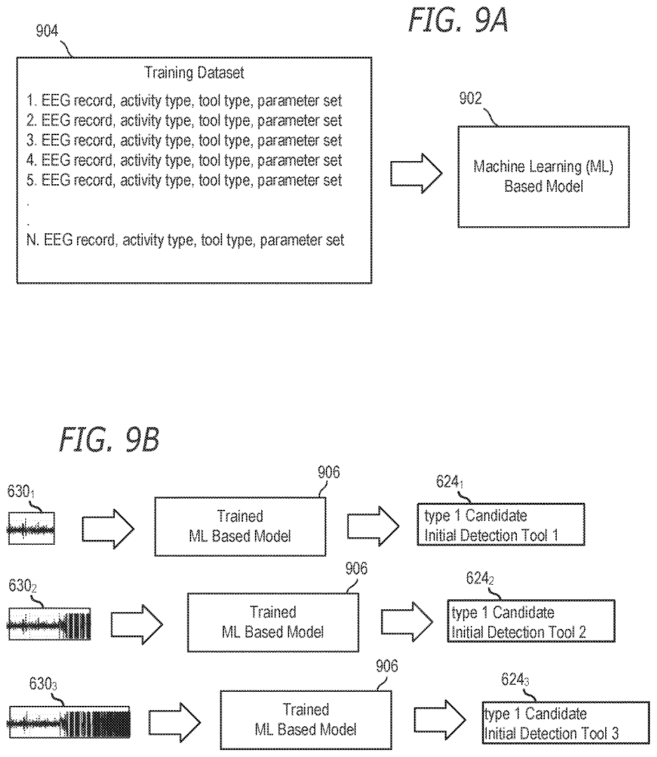

With reference to FIGS. 9A and 9B, in one embodiment, the CDT module 506 includes a machine learning based model 902 configured to determine candidate initial detection tools based on ROIs. The machine learning based model 902 is trained on a dataset 904 that includes EEG records with electrographic patterns or events that correspond to specific activity types, such as those listed in Table 4. The dataset also includes, for each EEG record, the activity type of the EEG record and information on the configuration of the detection tool that detected the pattern or event. The configuration information includes the type of detection tool and the detection parameter set of the detection tool. The configuration information can also include the size of the detection window. From this dataset 904, the machine learning based model 902 learns associations between electrographic patterns or events and activity types and detection tool configurations. In some embodiments, the machine learning based model 902 is of an architecture similar to the above disclosed ESC model.

With reference to FIG. 9B, the trained machine learning based model 906 is applied to each ROI 630₁, 630₂, 630₃, to provide a candidate initial detection tool 624₁, 624₂, 624₃ for that ROI, where each candidate initial detection tool is specified by configuration information, e.g., detection-tool type, detection parameter set, etc. Similarly, although not shown in FIG. 9B, the trained machine learning based model 906 is applied to each ROI 632₁, 632₂, 632₃, to provide a candidate initial detection tool 626₁, 626₂, 626₃ for that ROI.

In other embodiments, each type1 ROI 630₁, 630₂, 630₃ and each type2 ROI 632₁, 632₂, 632₃ is individually processed to determine a corresponding candidate parameter set, as disclosed in the following documents, each of which is hereby incorporated by reference: 1) U.S. Pat. No. 9,392,972, titled “Methods and Systems for Automatically Identifying Detection Parameters for an Implantable Medical Device,” 2) U.S. Pat. No. 10,722,176, titled “Methods and Systems for Automatically Identifying Detection Parameters for an Implantable Medical Device,” and 3) U.S. Pat. No. 10,729,352, titled “Neurological Event Detection Tools for Implantable Medical Devices.”

With reference to FIGS. 5B and 6E, the DTS module 508 receives and processes each type1-activity candidate initial detection tool 624₁, 624₂, 624₃ to obtain, for each candidate initial detection tool, metrics that represent the performance of the candidate initial detection tool for detecting electrographic activity corresponding to the type1-activity seizure onset. Similarly, the DTS module 508 receives and processes each type2-activity candidate initial detection tool 626₁, 626₂, 626₃ to obtain, for each candidate initial detection tool, metrics that represent the performance of the candidate initial detection tool for detecting electrographic activity corresponding to the type2-activity seizure onset.

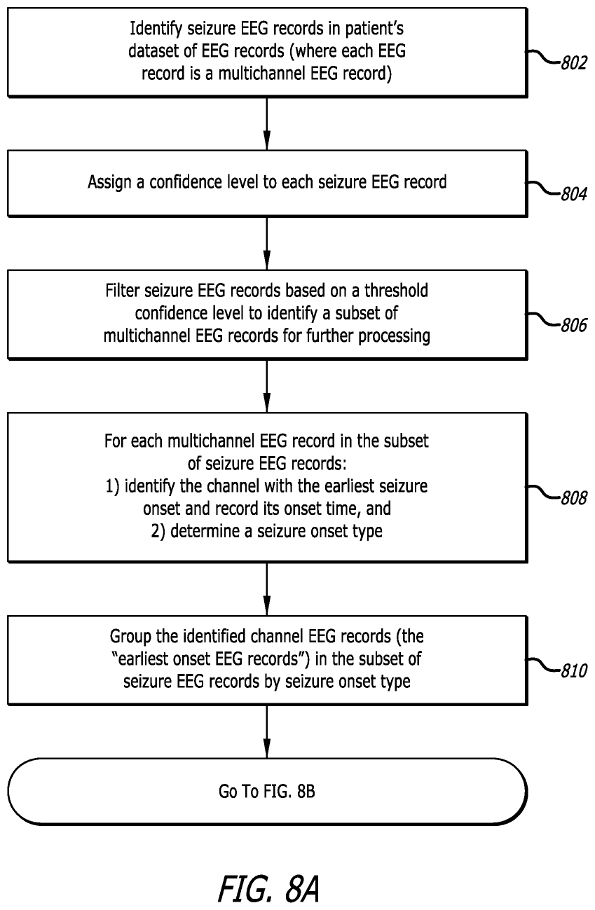

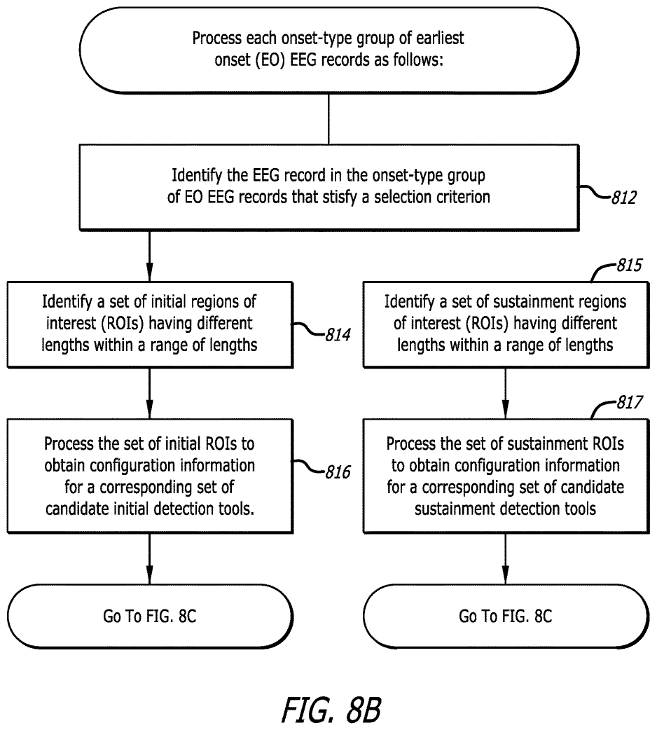

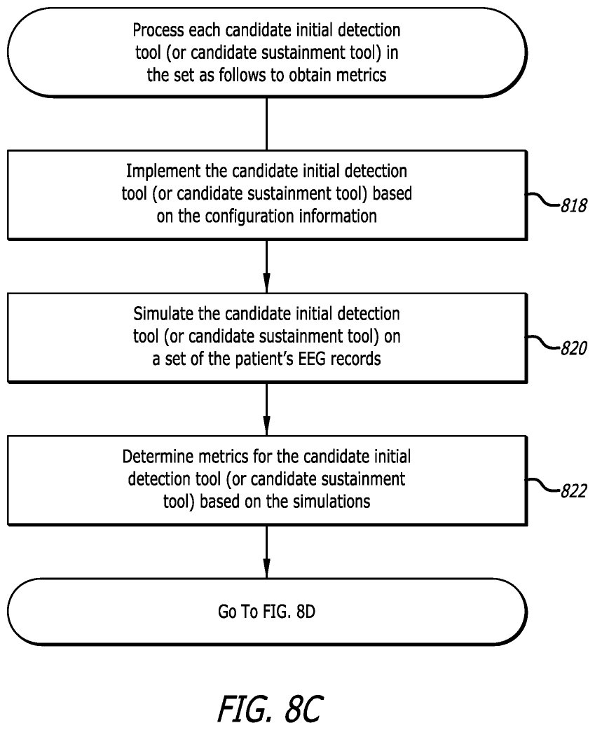

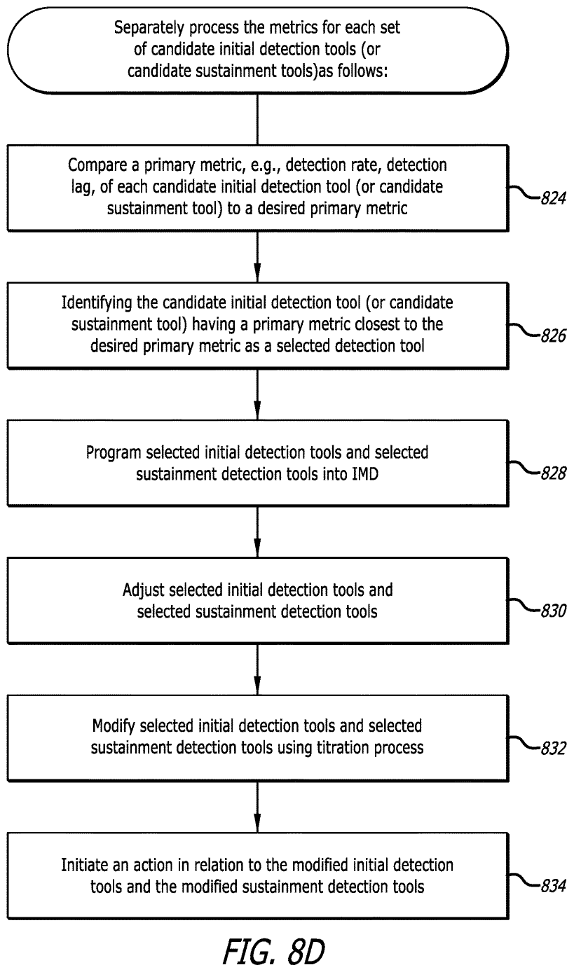

To these ends, the DST module 508 is configured to: 1) implement each candidate initial detection tool 624₁, 624₂, 624₃, 626₁, 626₂, 626₃ determined by the CDT module 506, 2) simulate each of the candidate initial detection tools by applying the tools to the same set of EEG records to obtain for each EEG record a simulation outcome, e.g., detections of abnormal activity, 3) determine a set of metrics for each candidate initial detection tool based on the simulation outcomes, and 4) process the respective metrics to identify a selected type1-activity initial detection tool 624 and a selected type2-activity initial detection tool 626. These functions of the DTS module 508 are disclosed below with reference to FIG. 8C, blocks 818 through 822 and FIG. 8D, blocks 824 through 830.

Regarding simulations of the candidate initial detection tools, the DTS module 508 applies each candidate initial detection tool 624₁, 624₂, 624₃, 626₁, 626₂, 626₃ to the same set of EEG records selected for simulation. In some embodiments, the set of EEG records selected for simulation is obtained from the EEG records dataset 510 and includes all of the EEG records recorded by the IMD 206 of the patient, or a subset thereof. In some embodiments, the set of EEG records selected for simulation includes a specific number of the most recently recorded records, or the records recorded within a specified time period. In some embodiments, the set of EEG records selected for simulation includes EEG records across a patient population having characteristics, e.g., demographics, age, lead location, lead type, seizure morphology, onset zone, type of brain abnormality, etc., in common with the patient.

With continued reference to FIGS. 5B and 6E, after the DTS module 508 identifies the selected initial detection tools 624, 626, it initiates an action in relation to the selected initial detection tool. In some embodiments, the action is implementing the selected initial detection tool 624, 626 in the patient's IMD 206.

For example, as shown in FIG. 5B, an type1-activity initial detection tool 514 of the detection subsystem 326 of the IMD 206 is implemented by programming a detection tool of the detection-tool type, e.g., halfwave detector, line length detector, specified by the configuration information 512 of the selected type1-activity initial detection tool 624 with the detection parameter set specified by the configuration information for the purposes of detecting type1-activity electrographic activity.

Similarly, a type2-activity initial detection tool 518 of the detection subsystem 326 of the IMD 206 is implemented by programming a detection tool of the detection-tool type, e.g., halfwave detector, line length detector, specified by the configuration information 516 of the selected type2-activity initial detection tool 626 with the detection parameter set specified by the configuration information for the purposes of detecting type2-activity electrographic activity.

The type1-activity initial detection tool 514 can be coupled to a sensing channel (also referred to as a detection channel) that is known to sense type1 activity. The type2-activity initial detection tool 518 can be coupled to a sensing channel that is known to sense type2 activity. In some embodiments, the sensing channel to which an initial detection tool 514, 518 is coupled corresponds to the channel that provided the EEG records 602 that were processed to determine the initial detection tool. In some embodiments, the type1-activity initial detection tool 514 and the type2-activity initial detection tool 518 can be coupled in parallel to a sensing channel that is known to sense electrical activity that includes a transition between type1 activity and type2 activity, such as shown in FIG. 7D.

With reference to FIG. 2, an alternative action of the detection proposer 202 is transmitting the configuration information 512 for type1 selected initial detection tool 624 and the configuration information 516 for the type2 selected initial detection tool 626 to a physician programmer 216 for storage and subsequent programming into the neurostimulator 302.

With reference to FIGS. 5A, 5B, 6F, 6G, and 6H, as previously mentioned, in some embodiments one or more sustainment ROIs are identified for each earliest onset EEG record 613, 615 and processed to propose a sustainment detection tool 714, 718 for the sensing channel to which an initial detection tool 514, 518 is coupled.

With reference to FIGS. 5A and 6F, the detection window module 504 is further configured to identify, for the selected type1-activity EO EEG record 613, a first set 640 of sustainment ROIs 650₁, 650₂, 650₃, each having a different length within a range of lengths, and to identify, for the selected type2-activity EO EEG record 615, a second set 642 of sustainment ROIs 652₁, 652₂, 652₃, each having a different length within a range of lengths. These sustainment ROIs may also be referred to as sustainment detection windows. Note, while the sets 640, 642 of sustainment ROIs in the example described herein include three sustainment ROIs, a set can include any number of ROIs and in some instances only one ROI.

In some embodiments, each sustainment ROI in a set of sustainment ROIs begins at a time after the initial ROI within the selected earliest onset EEG record 613, 615. In some embodiments, the begin time for a sustainment ROI is based on programmed settings. In one example, the begin time is in the range of 5-60 seconds after the initial ROI. In some embodiments, the respective durations of sustainment ROI in a set of sustainment ROIs are based on programmed settings. For example, the duration or period of time of the first sustainment ROI 650₁, is d, the duration of the second sustainment ROI 650₂, is a×d, and the duration of the third sustainment ROI 650₃, is b×d, where variables a, b and d are predetermined. In some embodiments, the range of lengths is based on the sustainment waveform type. Example ranges for sustainment ROI lengths are provided in Table 5 below:

TABLE 5

| Sustainment Waveform | Sustainment ROI length range (in seconds |
|---|---|
| Rhythmic Delta (D) | 2 to 4 |
| Rhythmic Theta (T) | 2 to 3 |
| Rhythmic Alpha (A) | 1.5 to 2.5 |
| Semi-Rhythmic Beta | 1.5 to 2.5 |
| (SRB) | |

With reference to FIG. 6G, the CDT module 506 is configured to determine a corresponding type1 candidate sustainment detection tool 644₁, 644₂, 644₃ based on each sustainment ROI 650₁, 650₂, 650₃ and a corresponding type2 candidate sustainment detection tool 646₁, 646₂, 646₃ based on each sustainment ROI 652₁, 652₂, 652₃. In some embodiments, the CDT module 506 is alternatively or additionally configured to determine a set of type1 candidate sustainment detection tools 644₁, 644₂, 644₃ based on sustainment ROIs taken from two or more records from set 612 of type1-activity earliest onset (EO) EEGs, and a corresponding set of type2 candidate sustainment detection tools 646₁, 646₂, 646₃ based on sustainment ROIs from two or more records from set 614 of type2-activity earliest onset (EO) EEGs. In some embodiments, the CDT module 506 is configured to determine one set of candidate sustainment detection tools 644₁, 644₂, 644₃ based on each sustainment ROI 650₁, 650₂, 650₃ for only one activity type. In some embodiments, the CDT module 506 is configured to determine more than two sets of candidate sustainment detection tools based on more than two activity types.

As an initial step in determining candidate sustainment detection tools, the CDT module 506 preprocesses each sustainment ROI to remove artifacts. This can be done in the same way as disclosed above for the candidate initial detection tools.

After preprocessing of the sustainment ROIs, each type1 sustainment ROI 650₁, 650₂, 650₃ is individually processed by the CDT module 506 to determine a corresponding activity-type 1 candidate sustainment detection tool 644₁, 644₂, 644₃. Likewise, each type2-activity sustainment ROI 652₁, 652₂, 652₃ is individually processed by the CDT module 506 to determine a corresponding type2 candidate sustainment detection tool 646₁, 646₂, 646₃. The respective sustainment detection tools are specified by configuration information that includes detection-tool type, e.g., halfwave, line length, etc., and a detection parameter set. This processing can be done in the same way as disclosed above for the candidate initial detection tools.

With reference to FIG. 6H, the DTS module 508 receives and processes each type1-activity candidate sustainment detection tool 644₁, 644₂, 644₃ to obtain, for each candidate sustainment detection tool, metrics that represent the performance of the candidate sustainment detection tool for detecting electrographic activity that is expected to follow a type1-activity seizure onset. This activity is referred to as sustainment activity and may correspond to a change in epileptiform activity that represents an evolution of a type1-activity seizure. Similarly, the DTS module 508 receives and processes each type2-activity candidate sustainment detection tool 646₁, 646₂, 646₃ to obtain, for each candidate sustainment detection tool, metrics that represent the performance of the candidate sustainment detection tool for detecting electrographic activity corresponding to sustainment activity that is expected to follow a type2-activity seizure onset.

To these ends, the DTS module 508 is configured to: 1) implement each candidate sustainment detection tool 644₁, 644₂, 644₃, 646₁, 646₂, 646₃ determined by the CDT module 506, 2) simulate each of the candidate sustainment detection tools by applying the tools to the same set of EEG records to obtain for each EEG record a simulation outcome, e.g., detections of sustainment activity, 3) determine a set of metrics for each candidate sustainment detection tool based on the simulation outcomes, and 4) process the respective metrics to identify a selected type1-activity sustainment detection tool 644 and a selected type2-activity sustainment detection tool 646. These functions of the DTS module 508 are disclosed below with reference to FIG. 8C, blocks 818 through 822 and FIG. 8D, blocks 824 through 830.

Regarding simulations of the candidate sustainment detection tools, the DTS module 508 applies each candidate sustainment detection tool 644₁, 644₂, 644₃, 646₁, 646₂, 646₃ to the same set of EEG records selected for simulation. In some embodiments, the set of EEG records selected for simulation is obtained from the EEG records dataset 510 and includes all of the EEG records recorded by the IMD 206 of the patient, or a subset thereof. In some embodiments, the set of EEG records selected for simulation includes a specific number of the most recently recorded records, or the records recorded within a specified time period. In some embodiments, the set of EEG records selected for simulation includes EEG records across a patient population having characteristics, e.g., demographics, age, lead location, lead type, seizure morphology, onset zone, type of brain abnormality, etc., in common with the patient.

With continued reference to FIG. 6H, after the DTS module 508 identifies the selected sustainment detection tools 644, 646, it initiates an action in relation to the selected sustainment detection tool. In some embodiments, the action is implementing the selected sustainment detection tool 644, 646 in the patient's IMD 206. For example, as shown in FIG. 5B, an type1-activity sustainment detection tool 714 of the detection subsystem 326 of the IMD 206 is implemented by programming a detection tool of the detection-tool type, e.g., halfwave detector, line length detector, specified by the configuration information 712 of the selected type1-activity sustainment detection tool 644, with the detection parameter set specified by the configuration information for the purposes of detecting sustainment activity of a type1-activity seizure.

Similarly, a type2-activity sustainment detection tool 718 of the detection subsystem 326 of the IMD 206 is implemented by programming a detection tool of the detection-tool type, e.g., halfwave detector, line length detector, specified by the configuration information 716 of the selected type2-activity sustainment detection tool 646, with the detection parameter set specified by the configuration information for the purposes of detecting sustainment activity of a type2-activity seizure.

The type1-activity sustainment detection tool 714 can be coupled to a sensing channel that is known to sense type1 activity. For example, the type1-activity sustainment detection tool 714 can be coupled in parallel with type1-activity initial detection tool 514. Coupled as such, in the presence of epileptiform activity corresponding to a type-1 activity seizure, the type1-activity initial detection tool 514 would detect the seizure onset and the type1-activity sustainment detection tool 714 would detect the evolution of the seizure. Likewise, the type2-activity sustainment detection tool 718 can be coupled to a sensing channel that is known to sense type2 activity. For example, the type2-activity sustainment detection tool 718 can be coupled in parallel with type2-activity initial detection tool 518.

FIGS. 8A-8D are a flowchart of a method of processing electrical activity of a brain to propose one or more detection tools for an IMD 206, where each detection tool is characterized by a detection-tool type and a set of detection parameters. In some embodiments, the method is performed by the detection proposer 202. In the following description the IMD 206 is configured to operate in either of an adaptive mode (during which therapy is not delivered by the IMD and detection tools are proposed and tuned) and a therapy mode (during which the IMD delivers therapy based on detections by the detection tools determined during the adaptive phase).

At block 802, and with reference to FIGS. 5A and 6A, the detection proposer 202 enters a first stage of operation while the IMD 206 is in adaptive mode. An ESC model 501 is applied to each of a plurality of EEG records 602, 604 of a brain included in a set 600 of records. In some embodiments, the simulation set of EEG records are records collected by an implanted neurostimulation system having detection tools programmed with default parameters. In some embodiments, each EEG record 602, 604 is a multichannel EEG record that includes a first channel record that corresponds to electrical activity sensed by a first sensing channel (CH1) of an implanted medical device, a second channel record that corresponds to electrical activity sensed by a second sensing channel (CH2) of the implanted medical device, a third channel record that corresponds to electrical activity sensed by a third sensing channel (CH3) of the implanted medical device, and a fourth channel record that corresponds to electrical activity sensed by a fourth sensing channel (CH4) of the implanted medical device. In some embodiments, the multichannel EEG record includes only two channels.

The ESC model 501 is configured to classify each of the plurality of multichannel EEG records 602, 604 as either a seizure record or a non-seizure record. As previously mentioned, in some embodiments the ESC model 501 is a machine learning based model trained in accordance with one of the models or a combination of models described in Barry et al. or Arcot Desai et al. In FIG. 6A, the multichannel EEG records 602 are classified as seizure records while the multichannel EEG records 604 are classified as non-seizure records. Thus, the method going forward is performed with respect to multichannel EEG records 602.

At block 804, a confidence level is assigned to each multichannel EEG record 602 that is classified as a seizure record. To this end, the ESC model 501 is further configured to assign a confidence level that represents the likelihood that a multichannel EEG record 602 classified as a seizure record is indeed a seizure record. For example, as mentioned previously, in some embodiments the ESC model 501 assigns a confidence level in the range of 0.1 to 0.9, where 0.1 indicates a low confidence level and 0.9 indicates a high confidence level. In other embodiments, the ESC model 501 assigns a confidence level in the range of 0.0 to 1.0, where 0.0 indicates the lowest confidence level and 1.0 indicates the highest confidence level.

At block 806, and with reference to FIGS. 5A and 6A, a set 606 of multichannel EEG records 602 classified as seizure records by the ESC model 501 is applied to a classification confidence filter 503. The classification confidence filter 503 is configured to filter the set 606 of multichannel seizure records 602 based on a threshold confidence level to provide a subset 608 of multichannel seizure records 602 for further processing. In some embodiments, the classification confidence filter 503 is programmed to include multichannel seizure records 602 with a confidence level at or above the threshold confidence level in the subset 608 of seizure records. The threshold confidence level is, for example, 0.8.

At block 808, and with reference to FIGS. 5A and 6B, the SOD module 601 of the EO/AT module 505 is configured to be applied to each multichannel seizure record 602 in the subset 608 of seizure records, to determine a seizure onset time 636a-d for each channel record 638a-d and to identify the channel record with the earliest seizure onset, and to record the time of that earliest seizure onset. Regarding FIG. 6B, for ease of illustration and description a single multichannel seizure record 602 is shown in detail. It is understood, however, that each multichannel seizure record 602 in the subset 608 of seizure records is processed by the EO/AT module 505. As previously mentioned, in some embodiments, the SOD module 601 comprises a convolutional neural network (CNN) based architecture. In other embodiments, the SOD module 601 is a deep learning model created using different methods, such as attention models (or other sequence based models) or signal processing algorithms. See, for example, “Recurrent Neural Networks for Forecasting Epileptiform Electrographic Activity 24 Hours in Advance,” AES abstract no. 2.050, published Nov. 6, 2018.

Also at block 808, and with reference to FIGS. 5A and 6B, the ATC module 603 of the EO/AT module 505 is configured to classify, for each multichannel seizure record 602 in the subset 608 of seizure records, a type or pattern of seizure onset. Example of seizure onset types are provided above in Table 3. As previously mentioned, in some embodiments the ATC module 603 invokes a clustering method in which features are extracted from the EEG records and passed through a clustering algorithm such as Bayesian Gaussian Mixture Models as disclosed in Barry et al. to determine which type/pattern (or cluster) of seizure onset the EEG records falls within. The ATC module 603 is trained to recognize various type/patterns of electrical activity in EEG records corresponding to seizure onsets. Some examples are shown in the table below along with a description. EEG examples with these activity patterns are further described in Nune et al., “Treatment of drug-resistant epilepsy in patients with periventricular nodular heterotopia using RNS System: Efficacy and description of chronic electrophysiological recordings,” Clinical Neurophysiology 2019, which is incorporated by reference.

Thus, the SOD module 601 and the ATC module 603 function together to identify, for each multichannel seizure record 602 in the subset 608 of seizure records, the channel EEG record 638 with the earliest seizure onset 636 (referred to as earliest onset EEG record) and for each earliest onset EEG record, an activity type or pattern of seizure onset. The collection of earliest onset EEG records 638 identified by the SOD module 601 and the ATC module 603 form a set 610 of earliest onset EEG records.

At block 810, and with reference to FIG. 6B, the EEG records in the set 610 of earliest onset EEG records 638 are grouped into “n” different subgroups based on seizure onset types. The subgroups includes a set 612 of type1-activity earliest onset (EO) EEGs, a set 614 of type2-activity EO EEGs through to a set 616 of type n EO EEGs. In one example, the type1-activity onset is a low voltage fast (LVF) seizure onset, while the type2-activity onset is a hypersynchronous seizure onset.

At block 812, and with continued reference to FIG. 6B, the set 612 of type1-activity EO EEGs, the set 614 of type2-activity EO EEGs through to the set 616 of type n EO EEGs are applied to a selection module 607 of the EO/AT module 505. The selection module 607 is configured to separately process each set 612, 614, 616 of earliest onset EEG records to identify, for each set, a selected EO EEG record 613, 615, 617 that meets a selection criterion. In some embodiments, the selection criterion is the EEG record with the earliest seizure onset time. Because the sets 612, 614, 616 of earliest onset EEGs records are grouped by seizure onset type, the selection module 607 can identify and select for each seizure onset type, the EEG record with the earliest seizure onset time as the selected EO EEG record 613, 615, 617. In other embodiments, the selection criterion is the EEG record that is most characteristic of the seizure onset type. To this end, the selection module 607 is configured to separately process each set 612, 614, 616 of earliest onset EEG records to identify, for each set, a selected EO EEG record 613, 615, 617 which is most characteristic of the set to which it belongs. In such embodiments, the selection module 607 may individually apply a clustering algorithm to each set 612, 614, 616 of earliest onset EEG records and select the centroid of the cluster as the selected EO EEG record 613, 615, 617 which is most characteristic of that seizure onset type.

At block 814, and with reference to FIGS. 5B and 6C, the detection window module 504 receives and separately processes the selected EO EEG records 613, 615. To this end, the detection window module 504 is configured to identify, for the selected type1-activity EO EEG record 613, a first set 620 of regions of interest (ROIs) 630₁, 630₂, 630₃, each having a different length within a range of lengths, and to identify, for a selected type2-activity EO EEG record 615, a second set 622 of ROIs) 632₁, 632₂, 632₃, each having a different length within a range of lengths. As disclosed above, in some embodiments, each ROI in a set of ROIs begins at the time of the seizure onset 636 within the selected EO EEG record 613, 615. In some embodiments, the respective durations of ROI in a set of ROIs is based on programmed settings. In some embodiments, the range of lengths is based on the seizure onset type of the selected EO EEG record.

At block 815, and with reference to FIGS. 5B and 6F, the detection window module 504 receives and separately processes the selected EO EEG records 613, 615. To this end, the detection window module 504 is configured to identify, for the selected type1-activity EO EEG record 613, a first set 640 of sustainment regions of interest (SROIs) 650₁, 650₂, 650₃, each having a different length within a range of lengths, and to identify, for a selected type2-activity EO EEG record 615, a second set 642 of SROIs 652₁, 652₂, 652₃, each having a different length within a range of lengths. As disclosed above, in some embodiments, each SROI in a set of SROIs begins at a time after the seizure onset 636 within the selected EO EEG record 613, 615. In some embodiments, the respective durations of SROI in a set of ROIs is based on programmed settings.

At block 816, and with reference to FIGS. 5B and 6D, the CDT module 506 receives and processes the selected type1 EO EEG record 613 based on the first set 620 of ROIs 630₁, 630₂, 630₃, to determine a set of type 1 candidate initial detection tools 624₁, 624₂, 624₃ corresponding in number to the number of ROIs 630₁, 630₂, 630₃. The CDT module 506 also receives and processes the selected type2 EO EEG record 615 based on the second set 622 of 632₁, 632₂, 632₃ to determine a set of type 2 candidate initial detection tools 626₁, 626₂, 626₃ corresponding in number to the number of ROIs 632₁, 632₂, 632₃. The sets of candidate detection tools is determined using one of the approaches described above.

At block 817, and with reference to FIGS. 5B and 6G, the CDT module 506 receives and processes the selected type1 EO EEG record 613 based on the first set 640 of SROIs 650₁, 650₂, 650₃, to determine a set of type 1 candidate sustainment detection tools 644₁, 644₂, 644₃ corresponding in number to the number of SROIs 650₁, 650₂, 650₃. The CDT module 506 also receives and processes the selected type2 EO EEG record 615 based on the second set 642 of SROIs 652₁, 652₂, 652₃ to determine a set of type 2 candidate sustainment detection tools 646₁, 646₂, 646₃ corresponding in number to the number of SROIs 652₁, 652₂, 652₃. The sets of candidate detection tools is determined using one of the approaches described above.

Proceeding to FIG. 8C, at blocks 818 through 822, and with reference to FIGS. 5B and 6E, the DTS module 508 receives and processes each type1 candidate initial detection tool 624₁, 624₂, 624₃ to obtain, for each candidate initial detection tool, metrics that represent the performance of the candidate initial detection tool for detecting electrographic activity corresponding to the type1-activity seizure onset. Similarly, the DTS module 508 receives and processes each type2 candidate initial detection tool 626₁, 626₂, 626₃ to obtain, for each candidate initial detection tool, metrics that represent the performance of the candidate initial detection tool for detecting electrographic activity corresponding to the type2-activity seizure onset.

More specifically, at block 818, the DTS module 508 implements a corresponding candidate initial detection tool for each candidate initial detection tool based on the configuration information provided by the CDT module 506. To this end, the DTS module 508 programs an initial detection tool of the type, e.g., half wave detection tool, line length detection tool etc., specified in the configuration information with the detection parameter set specified in the configuration information.

At block 820, for each candidate initial detection tool 624₁, 624₂, 624₃, 626₁, 626₂, 626₃, the DTS module 508 simulates the candidate initial detection tool on a simulation set of EEG records. In some embodiments, the EEG records in the simulation set include all or a subset of the patient's EEG records that are available in the EEG records dataset 510. In some embodiments, the simulation set of EEG records are records collected by an implanted neurostimulation system having detection tools programmed with default parameters.

At block 822, for each candidate initial detection tool 624₁, 624₂, 624₃, 626₁, 626₂, 626₃, the DTS module 508 determines a set of metrics for the candidate initial detection tool based on the simulations. The set of metrics can include one or more of the following:

Detection rate—the number of abnormal activity detections within a time period for a given patient. For example, for a patient having an implanted neurostimulation system that is known to deliver 2-3 stimulations per minute in response to a detection of a seizure onset, the target measure of detection rate is about 1,500-2,500 detections/day. A measure of detection rate, e.g., detections per hour, detections per day, etc., is obtained by simulating the candidate detection tool on a set of EEG records (including both seizure EEG records and non-seizure EEG records) of the patient that span a 24 hour period.

Detection lag—the average of time delays determined for a candidate detection tool for a set of seizure EEG records, where an individual time delay is determined for each seizure EEG record as the difference between the time of detection of an abnormal activity event as determined by the candidate detection tool and the time of the actual seizure onset as determined by a seizure onset detection model.

True positive rate—the percentage of true abnormal activity detections made by the candidate detection tool out of the total number of simulations on seizure EEG records (i.e., EEG records that are known to include a seizure onset). Sensitivity is measured by simulating the candidate detection tool on multiple seizure EEG records from the patient. It is expressed as a percentage and is also referred to as the true positive rate, reflecting the accuracy of the tool in detecting actual abnormal activity patterns

False positive rate—the percentage of abnormal activity detections made by the candidate detection tool on non-seizure EEG records (i.e., EEG records known to not include a seizure onset). This rate is calculated by simulating the candidate detection tool on multiple non-seizure EEG records from the patient. It represents the proportion of false detections and reflects how often the tool incorrectly identifies normal activity as abnormal.

Continuing with FIG. 8C, at blocks 818 through 822, and with reference to FIGS. 5B and 6H, the DTS module 508 receives and processes each type1 candidate sustainment detection tool 644₁, 644₂, 644₃ to obtain, for each candidate sustainment detection tool, metrics that represent the performance of the candidate sustainment detection tool for detecting electrographic activity expected to follow type1-activity seizure onset. Similarly, the DTS module 508 receives and processes each type2 candidate sustainment detection tool 646₁, 646₂, 646₃ to obtain, for each candidate sustainment detection tool, metrics that represent the performance of the candidate sustainment detection tool for detecting electrographic activity expected to follow the type2-activity seizure onset. In some embodiments, the candidate sustainment detection tools 644₁, 644₂, 644₃, 646₁, 646₂, 646₃ are processed in the same way as describe above for the candidate initial detection tools. Accordingly, the details of these block are not repeated here.

Proceeding to FIG. 8D, at block 824, the DTS module 508 compares a primary metric of each candidate initial detection tool 624₁, 624₂, 624₃ in a first set of candidate initial detection tools to a desired primary metric for those candidate initial detection tools. The DTS module 508 also compares a primary metric of each candidate initial detection tool 626₁, 626₂, 626₃ in a second set of candidate initial detection tools to a desired primary metric for those candidate initial detection tools. In some embodiments, the primary metric e.g., detection rate, detection lag, etc., and tolerance or measure of closeness is predetermined by the detection proposer 202. In some embodiments, the primary metric is input to the detection proposer 202 by a user through a user interface.

Continuing with FIG. 8D, block 824, the DTS module 508 also compares a primary metric of each candidate sustainment detection tool 644₁, 644₂, 644₃ in a first set of candidate sustainment detection tools to a desired primary metric for those candidate sustainment detection tools. The DTS module 508 also compares a primary metric of each candidate sustainment detection tool 646₁, 646₂, 646₃ in a second set of candidate sustainment detection tools to a desired primary metric for those candidate sustainment detection tools. In some embodiments, the primary metric e.g., detection rate, detection lag, etc., and tolerance or measure of closeness is predetermined by the detection proposer 202. In some embodiments, the primary metric is input to the detection proposer 202 by a user through a user interface.

In some embodiments, the primary metric is a detection rate corresponding to a number of detections within a certain time period. In some embodiments, the primary metric is a detection lag corresponding to the difference between a time of detection of an event by the candidate detection tool and a time of detection of the event by a seizure onset detection model. In some embodiments the primary metric for the first set of candidate detection tools is the same as the primary metric for the second set of candidate detection tools. In some embodiments the primary metric for the first set of candidate detection tools is different from the primary metric for the second set of candidate detection tools.

At block 826, the DTS module 508 identifies the candidate initial detection tool 624₁, 624₂, 624₃ having a primary metric that is closest to the desired primary metric as the selected type1-activity initial detection tool 624 for the first set of candidate detection tools. The DTS module 508 also identifies the candidate initial detection tool 626₁, 626₂, 626₃ having a primary metric that is closest to the desired primary metric as the selected type2-activity initial detection tool 626 for the second set of candidate initial detection tools.

In some embodiments, identification of a selected type1-activity initial detection tool 624 by the DTS module 508 comprises generating a new selected type1-activity initial detection tool 624 but is not constrained to be identical to one of the candidate initial detection tool 624₁, 624₂, 624₃, for example by using an optimization method such as simulated annealing, linear optimization, a genetic algorithm, or a machine learning algorithm seeded with one or more of the candidate initial detection tools 624₁, 624₂, 624₃ to generate a type1-activity initial detection tool 624 which yields a primary metric that is even closer to the desired primary metric than any of the candidate initial detection tools 624₁, 624₂, 624₃. Similarly, in some embodiments, identification of a selected type2-activity detection tool 626 by the DTS module 508 comprises generating a selected type2-activity initial detection tool 626 that is not constrained to be identical to one of the candidate initial detection tool 626₁, 626₂, 626₃.

At block 826, the DTS module 508 also identifies the candidate sustainment detection tool 644₁, 644₂, 644₃ having a primary metric that is closest to the desired primary metric as the selected type1-activity sustainment detection tool 644 for the first set of candidate detection tools. The DTS module 508 also identifies the candidate sustainment detection tool 646₁, 646₂, 646₃ having a primary metric that is closest to the desired primary metric as the selected type2-activity detection tool 646 for the second set of candidate sustainment detection tools.

In some embodiments, identification of a selected type1-activity sustainment detection tool 644 by the DTS module 508 comprises generating a new selected type1-activity sustainment detection tool 644 but is not constrained to be identical to one of the candidate sustainment detection tools 644₁, 644₂, 644₃, for example by using an optimization method such as simulated annealing, linear optimization, a genetic algorithm, or a machine learning algorithm seeded with one or more of the candidate sustainment detection tools 644₁, 644₂, 644₃ to generate a type1-activity sustainment detection tool 644 which yields a primary metric that is even closer to the desired primary metric than any of the candidate sustainment detection tools 644₁, 644₂, 644₃. Similarly, in some embodiments, identification of a selected sustainment detection tool 646 by the DTS module 508 comprises generating a selected type2-activity sustainment detection tool 646 that is not constrained to be identical to one of the candidate sustainment detection tools 646₁, 646₂, 646₃.

At block 828, the selected initial detection tools 624, 626 and the selected sustainment detection tools 644, 646 are programmed into an IMD 206. In some embodiments, the detection proposer 202 directly programs the selected initial detection tools 624, 626 and the selected sustainment detection tools 644, 646 into the IMD 206. For example, with reference to FIG. 5B, the configuration information for the selected type1-activity initial detection tool 624 can be used to configure a type1-activity initial detection tool 514 in the detection subsystem 326 of the IMD 206 for the purposes of detecting type1-activity electrographic activity. Similarly, the configuration information for the selected type2-activity initial detection tool 626 can be used to configure a type2-activity initial detection tool 518 in the detection subsystem 326 of the IMD 206 for the purposes of detecting type2-activity electrographic activity. Likewise, the configuration information for the selected type1-activity sustainment detection tool 644 can be used to configure a type1-activity sustainment detection tool 714 in the detection subsystem 326 of the IMD 206 for the purposes of detecting electrographic activity expected to follow a type-1-activity onset. Similarly, the configuration information for the selected type2-activity sustainment detection tool 646 can be used to configure a type2-activity sustainment detection tool 718 in the detection subsystem 326 of the IMD 206 for the purposes of detecting electrographic activity expected to follow a type2-activity onset.

In some embodiments, the detection proposer 202 transmits the configuration information for each of the selected type1-activity initial detection tool 624, the selected type2-activity initial detection tool 626, the type1-activity sustainment detection tool 644, and the type2-activity sustainment detection tool 646 to a physician programmer 216 (shown in FIG. 2) for storage and subsequent approval by a doctor and programming into an IMD 206. In either embodiment, once programmed the IMD 206 processes sensed EEG signals using the selected type1-activity initial detection tool 624, the selected type2-activity initial detection tool 626, the type1-activity sustainment detection tool 644, and the type2-activity sustainment detection tool 646. EEG signals that result in a detection by one or more of the selected type1-activity initial detection tool 624, the selected type2-activity initial detection tool 626, the type1-activity sustainment detection tool 644, and the type2-activity sustainment detection tool 646 are recorded by the IMD 206 and uploaded to the PDMS.

At block 830, subsequent to being programmed into the IMD 206, the settings or parameters of one or more of the programmed selected type1-activity initial detection tool 624, the selected type2-activity initial detection tool 626, the type1-activity sustainment detection tool 644, and the type2-activity sustainment detection tool 646 are coarsely adjusted. Going forward, these tools are referred to as “selected initial detection tools” or “selected sustainment detection tools.” In some embodiments, the DTS module 508 periodically performs a coarse adjustment of one or more of the selected initial detection tools 624, 626 and the selected sustainment detection tool 644, 646 as described in the following paragraphs, and automatically programs the IMD 206 with the coarse-adjusted detection tools. The process of block 830 corresponds to the end portion of stage 1 of the adaptive phase for the IMD 206.

In some embodiments, the DTS module 508 automatically performs a coarse adjustment of a selected initial detection tool 624, 626 (or selected sustainment detection tool 644, 646) as follows: data, e.g., detection counts and EEG records, collected as a result of detections by the selected detection tool are processed to obtain a primary metric; the primary metric is compared to a desired primary metric; and when the primary metric of the tool is not within a tolerance of the desired primary metric or does not satisfy a measure of closeness with the desired primary metric, the DTS module 508 adjusts one or more of the detection parameters of the tool. These are coarse adjustments of detection parameters because an expected change in a primary metric cannot be calculated based on knowledge of previous changes in the metric resulting from previous adjustments of detection parameters in this specific patient. After data have been uploaded to the PDMS 222 that show the change in a metric paired with a change in detection parameters, fine adjustments of detection parameters can be made using this information to target a specific metric.

For example, the primary metric can be sensitivity quantified in terms of detection rate, e.g., number of detections per day, that is based on the detection count, and the tolerance or measure of closeness can be a specified percentage, e.g., within 10%. In this case, if the DTS module 508 determines that a selected initial detection tool 624, 626 (or selected sustainment detection tool 644, 646) has a detection rate that is not within the specified percentage of the desired detection rate, the module applies a coarse adjustment to a parameter of the selected detection tool. This coarse adjustment is constrained to broad parameter changes. For example, the detection rate of a bandpass detection tool can be increased by adjusting the minimum HW amplitude parameter of the bandpass detection tool from 10% of the full-scale dynamic range of HW amplitudes to 5%. The change from 10% to 5% is considered a coarse adjustment. A change from 10% to any one of 4%, 3%, 2%, or 1% are also considered coarse adjustments.

In another example, the primary metric can be sensitivity quantified in terms of detection lag and the tolerance or measure of closeness can be a specified percentage, e.g., within 10%. In this case, EEG records included in the data are applied to the selected initial detection tool 624, 626 (or selected sustainment detection tool 644, 646) to obtain an individual detection lag for each EEG record. A measure, e.g., average detection lag, is derived and compared to the desired detection lag. If the DTS module 508 determines that a selected initial detection tool 624, 626 (or selected sustainment detection tool 644, 646) has a detection lag that is not within the specified percentage of the desired detection lag, the module applies a coarse adjustment to a parameter of the selected detection tool. This coarse adjustment is constrained to broad parameter changes. For example, the detection lag of a bandpass detection tool can be decreased by adjusting the minimum HW amplitude parameter of the bandpass detection tool from 10% of the full-scale dynamic range of HW amplitudes to 5%. The change from 10% to 5% is considered a coarse adjustment. A change from 10% to any one of 4%, 3%, 2%, or 1% are also considered coarse adjustments. The detection lag of a bandpass detection tool can also be decreased by adjusting the duration parameter of the bandpass detection tool from 256 to 512 msec.

In some embodiments, the DTS module 508 automatically performs a coarse adjustment of a selected initial detection tool 624, 626 (or selected a selected sustainment detection tool 644, 646) based on receipt of a target primary metric through a user interface. In this case, the target primary metric replaces or superseded the desired primary metric and the DTS module 508 performs a coarse adjustment regardless of how close the primary metric of a selected initial detection tool 624, 626 (or selected sustainment detection tool 644, 646) is to the desired primary metric. The coarse adjustments are performed the same as described above with the target primary metric used in place of the desired primary metric.

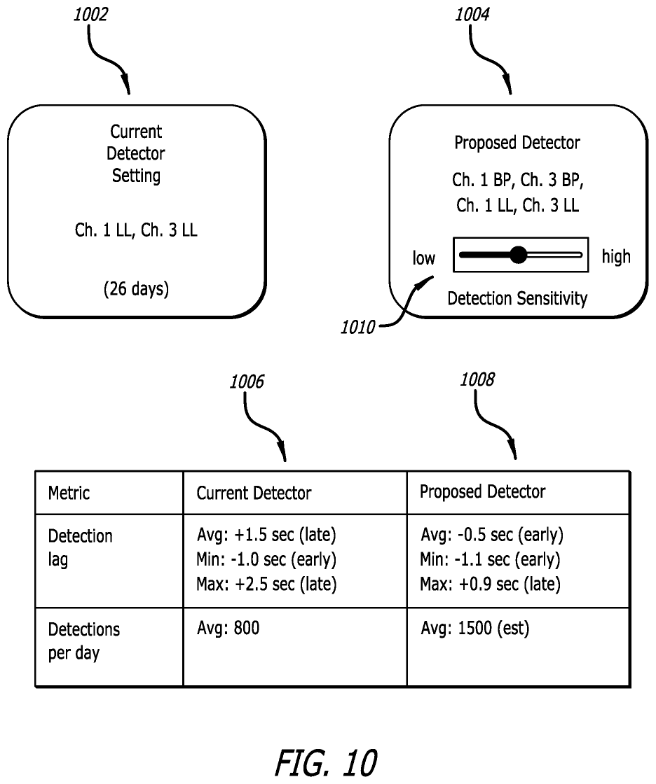

With reference to FIG. 10, in some embodiments the detection proposer 202 is configured to present current initial detection tool configuration information 1002, selected initial detection tool configuration information 1004, and their respective performance metrics 1006, 1008 on a display, together with a user interface 1010 through which a user can input a target primary metric for the selected initial detection tool that is being (or to be) adjusted. In the example of FIG. 10, the user interface 1010 enables a user to input a target primary metric corresponding to a value for detection rate through a slide bar. The detection proposer 202 can be configured to present similar information for sustainment detection tools.

At block 832, the detection proposer 202 transitions to a second stage of operation while the IMD 206 remains in adaptive mode. During this second stage, the DTS module 508 modifies (or fine tunes) one or more of the selected initial detection tools 624, 626 (or sustainment detection tool 644, 646) through a titration process. The detection tools that are modified by the process of block 832 correspond to the adjusted selected detection tools produced by the process of block 830. These “adjusted” selected detection tools are simply referred to as selected detection tools.

During this stage the DTS module 508 periodically, e.g., every 1 to 5 days, performs a cycle of a titration process as follows: data, e.g., detection counts and EEG records, collected as a result of detections by the selected detection tool (which at this point corresponds to coarse adjusted detection tool that results from block 830) are processed to obtain a primary metric; the primary metric is compared to a desired primary metric; and when the primary metric of the tool is not within a tolerance of the desired primary metric or does not satisfy a measure of closeness with the desired primary metric, the DTS module 508 titrates a detection parameter of the selected detection tool. Titrate in this context means the detection parameter is adjusted by a calculated amount that is informed by the results of one or more prior adjustment cycles. The adjustments to the detection parameters at this stage are on a smaller scale than the coarse adjustments and are thus referred to as fine adjustments.

Each cycle of the titration process comprises: (1) calculating a metric error, which is the difference between the current value of the performance metric (e.g., the observed detection rate) and the desired value of the performance metric (e.g., the target detection rate); (2) calculating a parameter delta, which is the difference between the current value of the detection parameter and the value of the detection parameter prior to the most recent adjustment; (3) using the metric error and the parameter delta together with the change in the performance metric that resulted from the most recent adjustment to estimate the amount of change to the detection parameter necessary to drive the performance metric toward the desired value; and (4) adjusting the detection parameter by the estimated amount and programming the adjusted detection tool into the IMD 206. After adjustment, new data (e.g., detection counts and EEG records) are accumulated over the next periodic interval, and the process repeats from step (1). Over multiple cycles, the performance metric converges toward the desired value.

The primary metric can be a detection rate, e.g., number of detections per day, that is based on the detection count, and the tolerance or measure of closeness can be a specified percentage, e.g., within 10%. In this case, if the DTS module 508 determines that a selected initial detection tool 624, 626 (or selected sustainment detection tool 644, 646) has a detection rate that is not within the specified percentage of the desired detection rate, the module titrates a parameter of the selected detection tool. For example, if the detection rate is 400 per day and the primary metric is 1000 detections per day, the measure of closeness is 10%, an adjustment would be made to the detection tool to increase the sensitivity. For a bandpass detector, this adjustment could be a decrease in the HW minimum amplitude by 1% to 5% of full-scale dynamic range. Considering this example further, if a bandpass detector's HW minimum amplitude was reduced from 56 to 48 counts in a prior cycle and the observed detection rate increased from 400 to 700 detections per day, the observed sensitivity of detection rate to amplitude change is approximately 37.5 detections per day per count ((700−400)/(56−48)). If the target detection rate is 1000 detections per day, the remaining metric error is 300 detections per day (1000-700), and the estimated amplitude reduction needed is 300/37.5=8 counts, yielding a new HW minimum amplitude of 40 counts. The adjusted detection tool is programmed into the IMD 206 and new detection data is collected over the next periodic interval.

The primary metric can be the detection lag and the tolerance or measure of closeness can be a specified percentage, e.g., within 10%. In this case, EEG records included in the data are applied to the selected initial detection tool 624, 626 (or selected sustainment detection tool 644, 646) to obtain an individual detection lag for each EEG record. A measure, e.g., average detection lag, is derived and compared to the desired detection lag. If the DTS module 508 determines that a selected initial detection tool 624, 626 (or selected sustainment detection tool 644, 646) has a detection lag that is not within the specified percentage of the desired detection lag, the module titrates a parameter of the selected detection tool. For example, if the detection lag is 2.0 seconds and the primary metric is 0.5 seconds, the measure of closeness is 10%, an adjustment would be made to the detection tool to increase the sensitivity. For a bandpass detector, this adjustment could be a decrease in the HW minimum amplitude by 1% to 5% of full-scale dynamic range. Considering this example further, if a bandpass detector's HW minimum amplitude was reduced from 56 to 48 counts in a prior cycle and the average detection lag decreased from 2.0 seconds to 1.2 seconds, the observed sensitivity of detection lag to amplitude change is approximately 0.1 seconds per count ((2.0−1.2)/(56−48)). If the target detection lag is 0.5 seconds, the remaining metric error is 0.7 seconds (1.2−0.5), and the estimated amplitude reduction needed is 0.7/0.1=7 counts, yielding a new HW minimum amplitude of 41 counts. The adjusted detection tool is programmed into the IMD 206 and new detection data is collected over the next periodic interval.

In the case of a selected initial detection tool 624, 626 (or selected sustainment detection tool 644, 646) corresponding to a half-wave (HW) detector having a detection parameter set comprising: a minimum frequency of 20.8 Hz (HW window size=220 ms, count criterion=10), a maximum frequency of 62.5 Hz (HW minimum width=4 ms), a HW minimum amplitude of 56 counts, a hysteresis of 32 counts, a minimum duration of 1.02 seconds (detect analysis window size=2048 msec, bandpass threshold=8), and a detection persistence of 1 second, the DTS module 508 titrates the selected detection tool as follows:

### Other Examples of Fine Adjustments are Presented Below:

To increase the detection rate, the DTS module 508 reduces the HW minimum amplitude from 56 to 48 counts. This would lead to more qualified half-waves per unit time and both earlier detection (reduced detection lag) and more frequent detection (higher detection rate). Alternatively, the bandpass threshold could be lowered from 8 to 7, thus requiring the qualified half-waves to be present for a shorter period of time. This would also result in both earlier and more frequent detection. Neither of these strategies would affect the frequency sensitivity of the half-wave detector. Applying the titration process described above to the HW minimum amplitude parameter: if in a prior titration cycle the HW minimum amplitude was reduced from 56 to 52 counts and the observed detection rate increased from 800 to 1000 detections per day, the observed sensitivity of detection rate to amplitude change is 50 detections per day per count ((1000−800)/(56−52)). If the target detection rate is 1200 detections per day and the current detection rate is 1000, the remaining metric error is 200 detections per day, and the estimated amplitude reduction needed is 200/50=4 counts, yielding a new HW minimum amplitude of 48 counts. The adjusted detection tool is programmed into the IMD 206 and new detection data is collected over the next periodic interval. The same titration calculation can be applied to the bandpass threshold parameter.

To decrease the detection rate, DTS module 508 increases the HW minimum amplitude from 56 to 64 counts. This would lead to fewer qualified half-waves per unit time and both later detection (increased detection lag) and less frequent detection (lower detection rate). Alternatively, the bandpass threshold could be increased from 8 to 9, thus requiring the qualified half-waves to be present for a longer period of time. This would also result in both later and less frequent detection. Neither of these strategies would affect the frequency sensitivity of the half-wave detector. Applying the titration process to decrease the detection rate: if in a prior titration cycle the HW minimum amplitude was increased from 56 to 60 counts and the observed detection rate decreased from 1500 to 1300 detections per day, the observed sensitivity of detection rate to amplitude change is 50 detections per day per count ((1500−1300)/(60−56)). If the target detection rate is 1000 detections per day and the current detection rate is 1300, the remaining metric error is 300 detections per day, and the estimated amplitude increase needed is 300/50=6 counts, yielding a new HW minimum amplitude of 66 counts. The adjusted detection tool is programmed into the IMD 206 and new detection data is collected over the next periodic interval.

In the case of a selected initial detection tool 624, 626 (or selected sustainment detection tool 644, 646) corresponding to a line length (LL) detector, having a detection parameter set comprising a detection threshold of 75%, a short-term trend of 2.05 seconds, a long-term trend of 2 minutes (window size=4096 msec, inter-sample interval=4096 msec, sample count=32), the DTS module 508 titrates the selected detection tool as follows:

To increase the detection rate, the DTS module 508 decreases the detection threshold from 75% to 62.5%. This would cause the LL detector to be triggered when the average LL during the short-term trend is greater than the long-term average LL by a smaller amount, resulting in both earlier detection and more frequent detection. Alternatively, the LL short-term trend could be reduced from 2.05 sec to 1.02 seconds. This would cause the LL detector to be triggered when the average short-term LL exceeds the detection threshold for a shorter period of time, resulting in both earlier and more frequent detection. Applying the titration process to the LL detection threshold parameter: if in a prior titration cycle the detection threshold was decreased from 75% to 68.75% and the observed detection rate increased from 600 to 850 detections per day, the observed sensitivity of detection rate to threshold change is 40 detections per day per percentage point ((850−600)/(75−68.75)). If the target detection rate is 1000 detections per day and the current detection rate is 850, the remaining metric error is 150 detections per day, and the estimated threshold reduction needed is 150/40=3.75 percentage points, yielding a new detection threshold of 65%. The adjusted detection tool is programmed into the IMD 206 and new detection data is collected over the next periodic interval. The same titration calculation can be applied to the short-term trend parameter.

To decrease the detection rate, the DTS module 508 increases the detection threshold from 75% to 87.5%. This would cause the LL detector to be triggered when the average LL during the short-term trend is greater than the long-term average LL by a greater amount, resulting in both later detection and less frequent detection. Alternatively, the LL short-term trend could be increased from 2.05 sec to 4.10 seconds. This would cause the LL detector to be triggered when the average short-term LL exceeds the detection threshold for a longer period of time, resulting in both later and less frequent detection. Applying the titration process to decrease the detection rate: if in a prior titration cycle the LL detection threshold was increased from 75% to 81.25% and the observed detection rate decreased from 1400 to 1150 detections per day, the observed sensitivity of detection rate to threshold change is 40 detections per day per percentage point ((1400−1150)/(81.25−75)). If the target detection rate is 1000 detections per day and the current detection rate is 1150, the remaining metric error is 150 detections per day, and the estimated threshold increase needed is 150/40=3.75 percentage points, yielding a new detection threshold of 85%. The adjusted detection tool is programmed into the IMD 206 and new detection data is collected over the next periodic interval.

In the case of a selected initial detection tool 624, 626 (or selected sustainment detection tool 644, 646) corresponding to an area-under-the-curve (Area) detector, having a detection parameter set that is the same as the LL detector example, i.e., a detection threshold of 75%, a short-term trend of 2.05 seconds, a long-term trend of 2 minutes (window size=4096 msec, inter-sample interval=4096 msec, sample count=32), the DTS module 508 modifies the selected detection tool in the same manner as the LL detector.

To increase the detection rate, the DTS module 508 decreases the detection threshold from 75% to 62.5%. This would cause the Area detector to be triggered when the average Area during the short-term trend is greater than the long-term average Area by a smaller amount, resulting in both earlier detection and more frequent detection. Alternatively, the Area short-term trend could be reduced from 2.05 sec to 1.02 seconds. This would cause the Area detector to be triggered when the average short-term Area exceeds the detection threshold for a shorter period of time, resulting in both earlier and more frequent detection. The titration calculation for the Area detector follows the same method as for the LL detector. For example, if in a prior titration cycle the detection threshold was decreased from 75% to 70% and the observed detection rate increased from 500 to 750 detections per day, the observed sensitivity of detection rate to threshold change is 50 detections per day per percentage point ((750−500)/(75−70)). If the target detection rate is 1000 detections per day and the current detection rate is 750, the remaining metric error is 250 detections per day, and the estimated threshold reduction needed is 250/50=5 percentage points, yielding a new detection threshold of 65%. The adjusted detection tool is programmed into the IMD 206 and new detection data is collected over the next periodic interval.

To decrease the detection rate, the DTS module 508 increases the detection threshold from 75% to 87.5%. This would cause the Area detector to be triggered when the average Area during the short-term trend is greater than the long-term average Area by a greater amount, resulting in both later detection and less frequent detection. Alternatively, the Area short-term trend could be increased from 2.05 sec to 4.10 seconds. This would cause the Area detector to be triggered when the average short-term Area exceeds the detection threshold for a longer period of time, resulting in both later and less frequent detection. Applying the titration process to decrease the detection rate: if in a prior titration cycle the Area detection threshold was increased from 75% to 80% and the observed detection rate decreased from 1300 to 1100 detections per day, the observed sensitivity of detection rate to threshold change is 40 detections per day per percentage point ((1300−1100)/(80−75)). If the target detection rate is 1000 detections per day and the current detection rate is 1100, the remaining metric error is 100 detections per day, and the estimated threshold increase needed is 100/40=2.5 percentage points, yielding a new detection threshold of 82.5%. The adjusted detection tool is programmed into the IMD 206 and new detection data is collected over the next periodic interval.

Additional information on adjusting detection parameter sets is provided in U.S. Pat. No. 8,131,352, titled “System and Method for Automatically Adjusting Detection Thresholds in a Feedback-controlled Neurological Event Detector,” which is incorporated by reference.

In some embodiments, the DTS module 508 automatically titrates a selected initial detection tool 624, 626 (or selected a selected sustainment detection tool 644, 646) based on receipt of a target primary metric through a user interface, such as described above with reference to FIG. 10. In this case, the target primary metric replaces or superseded the desired primary metric and the DTS module 508 performs titration regardless of how close the primary metric of a selected initial detection tool 624, 626 (or selected sustainment detection tool 644, 646) is to the desired primary metric. Titration is performed the same as described above with the target primary metric used in place of the desired primary metric.

At block 834, after titration of one or more of the selected initial detection tools 624, 626 (or selected sustainment detection tool 644, 646) is completed, the detection proposer 202 initiates an action in relation to each of the modified selected initial detection tools (herein after referred to simply as “modified initial detection tools”) or modified selected sustainment detection tools (herein after referred to simply as “modified sustainment detection tools”). In some embodiments, the detection proposer 202 directly programs the modified initial detection tools (and/or modified sustainment detection tools) into the IMD 206. In some embodiments, after programming the modified initial detection tools (and/or modified sustainment detection tools) into the IMD 206, the detection proposer 202 automatically enables therapy mode for the IMD; after which the IMD delivers therapy based on detections by the modified initial detection tools. In some embodiments, the detection proposer 202 transmits the configuration information for the modified initial detection tools (and/or modified sustainment detection tools) to a physician programmer 216 (shown in FIG. 2) for storage and subsequent programming into the IMD 206 by a doctor. Therapy mode for the IMD 206 can be enabled through the physician programmer 216.

### Concurrent Tuning of Detection Tools During Stimulation Therapy

As mentioned above, in some embodiments the detection proposer 202 determines and tunes detection parameters for one or more candidate detectors of the IMD 206 while the IMD is concurrently delivering stimulation therapy under the control of one or more active detectors. In such embodiments, the detection subsystem 326 of the IMD 206 includes a plurality of detector instances. A first subset of the detector instances are active detectors, each coupled to the therapy subsystem 328 such that detections by an active detector cause the therapy subsystem 328 to deliver electrical stimulation therapy in accordance with a stimulation parameter set. A second subset of the detector instances are candidate detectors, each not coupled to the therapy subsystem 328 such that detections by a candidate detector do not cause the therapy subsystem 328 to deliver electrical stimulation therapy. Each candidate detector is, however, coupled to the memory subsystem 338 such that detections by the candidate detector cause the memory subsystem 338 to increment a detection count and/or to store an EEG record of a corresponding EEG signal. The detection counts and EEG records are uploaded to the PDMS 222 for processing by the detection proposer 202.

In some embodiments, a candidate detector and an active detector are coupled in parallel to the same sensing channel of the IMD 206, such that the candidate detector and the active detector simultaneously process the same sensed electrographic activity. The candidate detector and the active detector can be of the same detection-tool type, e.g., both are halfwave detectors, both are line length detectors, etc., with the candidate detector being defined by a different parameter set than the active detector. Alternatively, the candidate detector and the active detector can be of different detection-tool types, e.g., the active detector is a halfwave detector and the candidate detector is a line length detector. In some embodiments, a candidate detector is coupled to a different sensing channel than any active detector. In some embodiments, the candidate detectors include a candidate initial detection tool and a candidate sustainment detection tool corresponding to the initial and sustainment detection tools described above, with the candidate sustainment detection tool configured to detect a pattern indicative of electrographic activity that follows an onset pattern detected by the candidate initial detection tool. In some embodiments, the role of a detector instance as an active detector or a candidate detector changes over time, including embodiments in which the detection subsystem 326 of the IMD 206 includes only a single detector instance whose role changes between the candidate-detector role and the active-detector role.

The detection proposer 202 determines and tunes detection parameters for the one or more candidate detectors using the methods disclosed above with reference to FIGS. 5A-8D, with detection counts and EEG records collected by the candidate detectors substituted for the detection counts and EEG records that, in the pre-therapy embodiments described above, are collected by the IMD 206 during the adaptive phase. In some embodiments, the candidate detectors are initialized with a default parameter set, with a parameter set determined by the detection proposer 202 from EEG records collected by the IMD 206 before the candidate detectors were instantiated, or with a parameter set copied from an active detector currently coupled to the therapy subsystem 328. The detection proposer 202 then performs the first stage of the adaptive phase as disclosed above to coarsely adjust the parameters of each candidate detector, and the second stage of the adaptive phase as disclosed above to titrate the parameters of each candidate detector toward a target primary metric, e.g., a target detection rate or a target detection lag. Throughout this process, the active detectors continue to govern the delivery of stimulation therapy by the therapy subsystem 328, and the parameters of the active detectors are not modified by the operations of the detection proposer 202 on the candidate detectors.

After the detection parameters of a candidate detector have been determined and tuned, the detection proposer 202 evaluates one or more handover criteria to determine whether the candidate detector should replace, or have its configuration information transferred to, an active detector. Examples of handover criteria include, alone or in combination: (1) a primary metric of the candidate detector is within a tolerance of a desired primary metric, e.g., the candidate detector's detection rate is within a specified percentage of a target detection rate; (2) the primary metric of the candidate detector is closer to the desired primary metric than the corresponding primary metric of an active detector currently coupled to the therapy subsystem 328; (3) the primary metric of the candidate detector has remained within the tolerance of the desired primary metric across two or more consecutive evaluation intervals; and (4) the candidate detector has detected a specified minimum number of events, or has been operating for a specified minimum period of time, since the most recent adjustment of its detection parameters.

In response to satisfaction of the one or more handover criteria, in response to an input received from a physician or other user through a user interface, or both, the detection proposer 202 initiates a handover by which the tuned candidate detector becomes responsible for governing delivery of stimulation therapy. In some embodiments, the handover is performed by transferring the detection parameter set, and optionally the detection-tool type, of the tuned candidate detector to an active detector, such that the active detector subsequently operates with the parameter set previously associated with the candidate detector. In other embodiments, the handover is performed by re-designating the tuned candidate detector as an active detector, e.g., by routing detections of the tuned candidate detector to the therapy subsystem 328, and optionally re-designating the previously active detector as a candidate detector, as a non-active detector, or removing it from operation.

In some embodiments, the handover is performed atomically with respect to delivery of stimulation therapy, such that delivery of stimulation therapy continues without interruption across the handover. In some embodiments, the detection proposer 202 directly programs the IMD 206 to effect the handover. In some embodiments, the detection proposer 202 transmits the configuration information for the tuned candidate detector to the physician programmer 216 for storage and subsequent approval by a physician through a user interface, such as the user interface described with reference to FIG. 10, prior to programming of the handover into the IMD 206. In some embodiments, after the handover, the detection proposer 202 continues to monitor a primary metric for the now-active detector and, in response to a determination that the primary metric has departed from the desired primary metric by more than a threshold amount, the detection proposer 202 reverts the IMD 206 to a prior parameter set, e.g., the parameter set of the previously active detector, or initiates a new tuning cycle on a candidate detector.

In some embodiments, a handover is initiated by a physician or other user without regard to whether the handover criteria evaluated by the detection proposer 202 have been satisfied. To this end, the physician programmer 216 includes a user interface configured to present configuration information and one or more performance metrics for each of one or more candidate detectors and for each of one or more active detectors currently coupled to the therapy subsystem 328. The configuration information presented for a detector can include the detection-tool type, e.g., halfwave, line length, area, etc., and the detection parameter set defining the detector. The performance metrics presented for a detector can include detection rate, detection lag, true positive rate, false positive rate, and any other metric described herein, and can be presented for each of one or more time intervals, e.g., the most recent 24 hours, the most recent 7 days, the period since the most recent parameter adjustment, etc.

In some embodiments, the user interface presents the configuration information and performance metrics for a candidate detector alongside the corresponding configuration information and performance metrics for an active detector to enable side-by-side comparison. In some embodiments, the user interface enables the user to select, from among a plurality of candidate detectors, a particular candidate detector to be the subject of a handover. In some embodiments, the user interface enables the user to modify the detection parameter set of the selected candidate detector prior to handover, and the modified parameter set is the parameter set programmed into the IMD upon handover. In response to a user input through the user interface confirming a handover, the physician programmer 216 effects the handover by transferring the configuration information of the selected candidate detector to an active detector, by re-designating the selected candidate detector as an active detector, or by another mechanism described above. In some embodiments, the user interface is presented on the physician programmer 216. In other embodiments, the user interface is presented on a different device in communication with the PDMS 222, such as a clinician-facing interface accessible through a network 208, or, with appropriate authorization, on the remote patient monitor 210, 1206. A handover initiated through the user interface can be performed at any time during the adaptive phase or during therapy delivery, including before, after, or in the absence of satisfaction of the handover criteria evaluated by the detection proposer 202.

Concurrent tuning of candidate detectors during stimulation therapy can be invoked in any of a variety of clinical circumstances. Examples include, without limitation: a change over time in the patient's electrographic activity, e.g., emergence of a new seizure onset type or evolution of an existing onset morphology, that suggests the parameter set of an active detector is no longer optimal; a determination by the detection proposer 202 or by a physician that the performance of an active detector has departed from a desired primary metric; addition of a sustainment detection tool to a patient who was previously implanted with only an initial detection tool, or vice versa; comparison of candidate detectors of different detection-tool types, e.g., a halfwave detector and a line length detector, on the same sensing channel to identify the type that yields the best primary metric for the patient; receipt of a new target primary metric, e.g., a new target detection rate, through the programmer 216; and periodic re-tuning of detection parameters at scheduled intervals to maintain a desired primary metric in the presence of physiological drift. In any such circumstance, the patient continues to receive stimulation therapy under the control of the existing active detector during the determination and tuning of the candidate detector, with the handover occurring only after the handover criteria are satisfied.

Because the candidate detector is not coupled to the therapy subsystem 328, the determination and tuning of the candidate detector by the detection proposer 202 does not modify the patient's ongoing stimulation therapy. The patient's stimulation therapy is governed throughout this process by the active detector and by the stimulation parameters associated with the active detector, neither of which is modified as a result of the determination and tuning of the candidate detector. Accordingly, the methods disclosed herein for determining and tuning detection parameters can be performed during a post-operative period prior to therapy activation, as described above with reference to FIGS. 8A-8D, and can also be performed during therapy delivery, with the handover occurring only after the candidate detector's performance has been validated against the handover criteria.

In some embodiments, an active detector currently coupled to the therapy subsystem 328 is itself tuned in situ by the detection proposer 202 while the active detector continues to govern delivery of stimulation therapy. To this end, the detection proposer 202 adjusts one or more detection parameters of the active detector by an amount bounded by a safety constraint, e.g., a maximum allowable change per adjustment cycle, a maximum allowable cumulative change across two or more adjustment cycles, or a constraint that the adjusted detection parameter remain within a predetermined range. Stimulation therapy continues to be delivered in response to detections by the active detector throughout the adjustment, and the adjusted active detector remains coupled to the therapy subsystem 328 after the adjustment. After the adjustment, the detection proposer 202 collects new data, e.g., detection counts and EEG records, that result from detections by the active detector with the adjusted parameter, processes the new data to obtain an updated primary metric for the active detector, and repeats the adjustment as needed to drive the primary metric toward a desired primary metric. In some embodiments, the in-situ adjustment of the active detector is performed using the titration process described above with reference to FIG. 23 and the second stage of the adaptive phase. In some embodiments, an in-situ active detector tuning process as described in this paragraph is performed in combination with a candidate detector tuning process as described above, for example by validating a tentative parameter adjustment on a candidate detector before applying the same adjustment to the active detector.

In some embodiments, one or more of the operations described herein for proposing, coarsely adjusting, titrating, or otherwise tuning a detection tool, including any of the operations described with reference to FIGS. 5A-8D, are performed by the detection proposer 202 automatically. In other embodiments, one or more of those operations are performed by a human reviewer, e.g., a clinician, clinical engineer, case analyst, technician, or other user, through a case analyst interface, with the remaining operations performed automatically by the detection proposer 202. The division of labor between the human reviewer and the detection proposer 202 can be configured to allocate any one or more individual operations, or any contiguous portion of either or both stages of the adaptive phase, to the human reviewer.

In one embodiment, the human reviewer performs the operations of the first stage of the adaptive phase, including determining one or more initial detection tools, optionally determining one or more sustainment detection tools, and coarsely adjusting the detection parameters of the determined detection tools, while the detection proposer 202 thereafter automatically performs the operations of the second stage of the adaptive phase, including titration of the detection parameters as disclosed herein.

In another embodiment, the detection proposer 202 automatically performs a portion of the first stage, e.g., classification of EEG records by the ESC model 501 and identification of earliest-onset records and activity types by the EO/AT module 505, and the human reviewer performs the remaining portion of the first stage, e.g., selection of the ROIs, selection of the detection-tool type, and specification of the detection parameter set, after which the detection proposer 202 automatically performs the second stage.

In another embodiment, the human reviewer performs only a subset of the operations of the first stage, with the detection proposer 202 performing the remaining operations of the first stage and all operations of the second stage. In yet another embodiment, the human reviewer reviews and accepts, rejects, or modifies one or more candidate detection tools 624₁-624₃, 626₁-626₃, 644₁-644₃, 646₁-646₃ proposed by the CDT module 506, and the detection proposer 202 thereafter automatically performs the remaining operations.

In some embodiments, the case analyst interface is presented on the physician programmer 216, on the PDMS 222, or on a separate workstation in communication with the PDMS 222 through the network 208. The case analyst interface is configured to present any of the information described herein with respect to the detection proposer 202, including without limitation: the EEG records dataset 510 of the patient, classifications of EEG records as seizure or non-seizure records, confidence levels associated with such classifications, predicted seizure onset times on a per-channel basis, activity-type classifications, candidate sets of ROIs, candidate detection tools and their associated configuration information, and performance metrics, e.g., detection rate, detection lag, true positive rate, false positive rate, of any candidate or selected detection tool when simulated on the EEG records dataset 510.

The case analyst interface is further configured to receive inputs from the human reviewer that specify, modify, or override any of the foregoing, including without limitation inputs that select an EEG record from the EEG records dataset 510 as a selected EO EEG record 613, 615, 617; that specify the start time, duration, or both of an ROI 630, 632 or sustainment ROI 650, 652; that specify the detection-tool type and detection parameter set of a candidate or selected detection tool; that accept, reject, or modify a candidate or selected detection tool proposed by the detection proposer 202; that initiate a simulation of a specified detection tool on a specified set of EEG records and present the resulting performance metrics; and that commit a specified detection tool, with a specified detection parameter set, to be programmed into the IMD 206 as an initial detection tool 514, 518, a sustainment detection tool 714, 718, an active detector, or a candidate detector.

In some embodiments, the detection proposer 202 is configured to detect a readiness condition and, in response to detection of the readiness condition, to generate a notification that is presented through the case analyst interface, the physician programmer 216, the remote patient monitor 210, 1206, or another communication channel. The notification indicates that sufficient information has been collected for the human reviewer to perform one or more operations of the adaptive phase. Examples of readiness conditions include, without limitation: a specified number of seizure EEG records have been collected from the patient by the IMD 206; a specified number of EEG records satisfying a confidence-level threshold have been classified as seizure records by the ESC model 501; a specified period of time has elapsed since implant of the neurostimulation system 206; a specified period of time has elapsed since the most recent adjustment of a detection parameter; the EEG records dataset 510 contains EEG records representing a specified number of distinct seizure onset types or activity types as classified by the ATC module 603; a primary metric of an active detector has departed from a desired primary metric by more than a threshold amount; and any combination of the foregoing.

In response to receipt of inputs through the case analyst interface that commit one or more detection tools determined by the human reviewer, the detection proposer 202 automatically programs the committed detection tools into the IMD 206 and, where applicable, automatically initiates the second stage of the adaptive phase to titrate the detection parameters of the committed detection tools as disclosed herein. In some embodiments, the human reviewer can intervene through the case analyst interface during operations otherwise performed automatically by the detection proposer 202, including during the second stage of the adaptive phase or during concurrent tuning of candidate detectors during stimulation therapy, to inspect, modify, accept, or reject any candidate or selected detection tool, any detection parameter, or any performance metric.

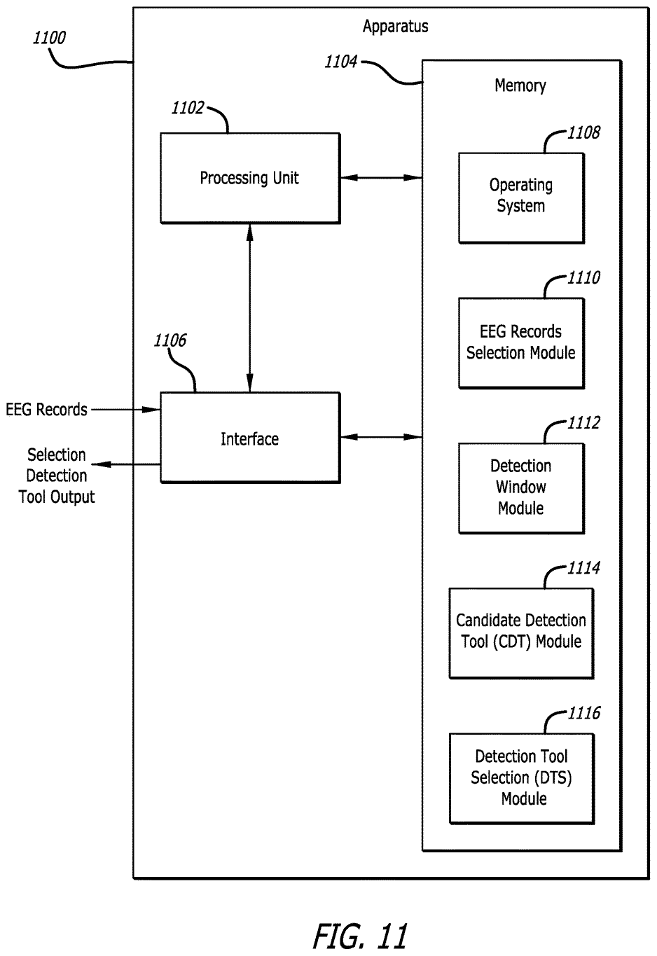

FIG. 11 is a schematic block diagram of an apparatus 1100 corresponding to the detection proposer 202 of FIG. 2. The apparatus 1100 is configured to execute instructions related to the detection set processes described above with reference to FIGS. 5A-8D. The apparatus 1100 is embodied in any number of processor-driven devices, including, but not limited to, a server computer, a personal computer, one or more networked computing devices, an application-specific circuit, a minicomputer, a microcontroller, and/or any other processor-based device and/or combination of devices.

The apparatus 1100 includes one or more processing units 1102 configured to access and execute computer-executable instructions stored in at least one memory 1104. The processing unit 1102 is implemented as appropriate in hardware, software, firmware, or combinations thereof. Software or firmware implementations of the processing unit 1102 can include computer-executable or machine-executable instructions written in any suitable programming language to perform the various functions described herein. The processing unit 1102 can include, without limitation, a central processing unit (CPU), a digital signal processor (DSP), a reduced instruction set computer (RISC) processor, a complex instruction set computer (CISC) processor, a microprocessor, a microcontroller, a field programmable gate array (FPGA), a System-on-a-Chip (SOC), or any combination thereof. The apparatus 1100 can also include a chipset (not shown) for controlling communications between the processing unit 1102 and one or more of the other components of the apparatus. The processing unit 1102 can also include one or more application-specific integrated circuits (ASICs) or application-specific standard products (ASSPs) for handling specific data processing functions or tasks.

The memory 1104 can include, but is not limited to, random access memory (RAM), flash RAM, magnetic media storage, optical media storage, and so forth. The memory 1104 can include volatile memory configured to store information when supplied with power and/or non-volatile memory configured to store information even when not supplied with power. The memory 1104 can store various program modules, application programs, and so forth that can include computer-executable instructions that upon execution by the processing unit 1102 can cause various operations to be performed. The memory 1104 can further store a variety of data manipulated and/or generated during execution of computer-executable instructions by the processing unit 1102.

The apparatus 1100 can further include one or more interfaces 1106 that can facilitate communication between the apparatus and one or more other apparatuses. For example, the interface 1106 is configured to receive EEG records from a database and to output information, e.g., therapy suggestions, to a display. Communication is implemented using any suitable structure or standard of communication. For example, communication with a database is through a LAN interface that implement protocols and/or algorithms that comply with various communication standards of the Institute of Electrical and Electronics Engineers (IEEE), such as IEEE 802.11. Communication with a user interface and display may be through wired or wireless means.

The memory 1104 can store various program modules, application programs, and so forth that can include computer-executable instructions that upon execution by the processing unit 1102 can cause various operations to be performed. For example, the memory 1104 can include an operating system module (O/S) 1108 that is configured to manage hardware resources such as the interface 1106 and provide various services to applications executing on the apparatus 1100.

The memory 1104 stores additional program modules such as: (1) an EEG records selection module 1110; (2) a detection window module 1112; (3) candidate detection tool (CDT) module 1114; and (4) a DTS module 1116. Each of these modules includes computer-executable instructions that when executed by the processing unit 1102 cause various operations to be performed, including the operations described above with reference to FIGS. 5A-8D.

The apparatus 1100 and modules disclosed herein are implemented in hardware or software that is executed on a hardware platform. The hardware or hardware platform is a general purpose processor, a digital signal processor (DSP), an application specific integrated circuit (ASIC), a field programmable gate array (FPGA) or other programmable logic component, discrete gate or transistor logic, discrete hardware components, or any combination thereof, or any other suitable component designed to perform the functions described herein. A general-purpose processor is a microprocessor, but in the alternative, the processor is any conventional processor, controller, microcontroller, or state machine. A processor can also be implemented as a combination of computing components, e.g., a combination of a DSP and a microprocessor, a plurality of microprocessors, one or more microprocessors in conjunction with a DSP, or any other such configuration.

Systems and/or methods as described in this disclosure may use detection tools other than or in addition to half wave, line length, and area detection tools. Such detection tools may include, for example, detection tools based on the following parameters calculated from EEG data: root-mean-square (RMS) amplitude; amplifier saturation or other thresholding; aperiodic exponent; coherence; correlation; covariance; signal power calculated by fast Fourier transform, wavelet transform, or other means in a frequency band such as the delta, alpha, beta, or gamma band of EEG; or the ratio of signal power calculated in frequency bands such as power in the beta band of EEG divided by power in the gamma band of EEG. Such detection tools may identify a change in a calculated parameter, for instance by using the short-term and long-term trend mechanism described previously in this disclosure. Such detection tools may include recurrent neural network (RNN) models such as convolutional neural networks (CNNs), long short-term memory (LSTM) networks, vision transformers, or other models configured to detect abnormal electrographic activity while operating efficiently in a low-power, low-memory implantable embedded system.

In some embodiments, steps of the systems and methods described in this disclosure may be combined; for example, identification and filtering of seizure EEG records in blocks 802, 804, and 806 may be performed implicitly by a neural network which identifies seizure onset times and determines seizure onset type in block 808, for example by configuring the EO/AT model 505 used in block 808 to yield seizure onset channel, time, and activity type for records which have electrographic seizures, and a determination of no electrographic seizure for records which do not have electrographic seizures.

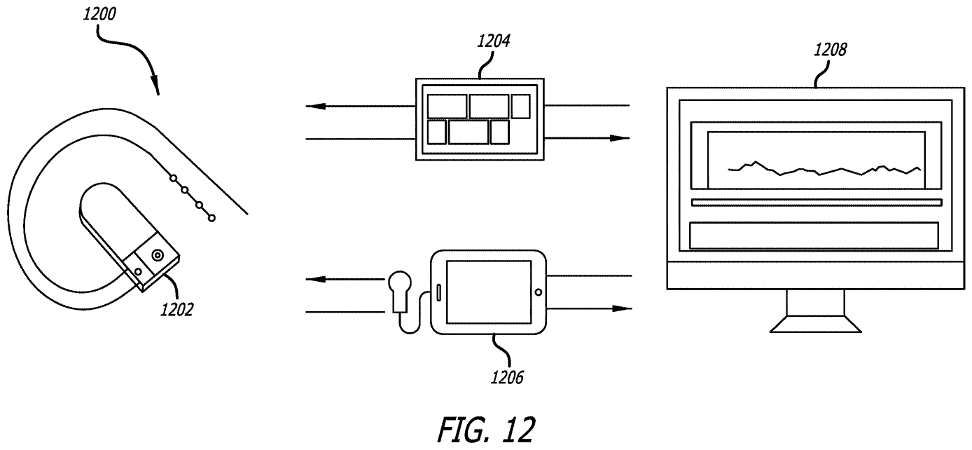

FIG. 12 is an illustration of a system 1200 similar to the system shown in FIG. 2. The system 1200 includes an implantable medical device (IMD) 1202, a physician programmer 1204, a remote patient monitor 1206, and a patient data management system (PDMS) 1208. In some embodiments the IMD 1202 is a neurostimulation system having a neurostimulator connected to leads, as disclosed herein. In some embodiments, the neurostimulator is configured as described with reference to FIG. 4. Accordingly, the neurostimulator has a detection subsystem comprising one or more detection tools that can be programmed to detect specific patterns in EEG signals using the detection tools describe earlier in this disclosure.

The PDMS 1208 includes a cloud-based data repository with machine learning features that can analyze data and make recommendations for detection setting changes automatically. The physician programmer 1204 includes a computer used by the physician that uses wireless telemetry to communicate with the neurostimulation system 1202. The physician programmer 1204 can retrieve information from the neurostimulation system 1202 and program settings in the neurostimulation system. The remote patient monitor 1206 includes a computer used by patients that uses wireless telemetry to communicate with the neurostimulation system 1202. The remote patient monitor 1206 can retrieve information and program settings in the neurostimulation system 1202 remotely as directed by the physician or automatically by the PDMS 1208 during a detection tool setting period.

### Sequence for Automatically Setting Detection Tools:

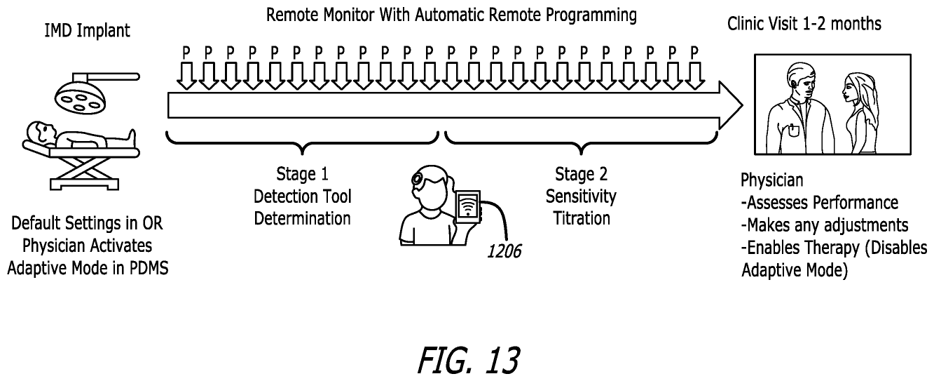

FIG. 13 is an illustration of a sequence for implanting and automatically programming detection tools of a neurostimulation system 1202. Starting from the left of FIG. 13, a patient is implanted with a neurostimulation system 1202. At implant, the detection tools of the neurostimulator are programmed with default detection settings. The patient is discharged with a remote patient monitor 1206. The remote patient monitor 1206 operates in conjunction with the PDMS 1208 to performs a method that automatically proposes, adjusts, and optimizes detection tools for the patient. The method includes the following two stages:

Stage 1—Detection Tool Determinations: During this stage, patient data, e.g., EEG records, are collected and detection tools for detecting one or more types of electrographic activity types (e.g., gamma detection) are periodically determined by the PDMS 1208. In some embodiments, the periodic determination or selection of detection tools occurs every 1 to 3 days. The remote patient monitor 1206 receives information from the PDMS 1208 that define the determined or selected detection tools, and programs the neurostimulation system 1202 with the selected detection tools.

Stage 2—Sensitivity Titration: During this stage, the settings for detection tools selected during Stage 1 and programmed into the neurostimulation system 1202 are periodically adjusted by the PDMS 1208 using a titration process to achieve a target detection rate. In some embodiments, the periodic adjustment of detection tools occurs every 1 to 3 days. The remote patient monitor 1206 receives information from the PDMS 1208 that define the adjusted/modified detection tools, and programs the neurostimulation system 1202 with the adjusted/modified detection tools.

Next, at a clinic visit, for example in 1-3 months after the implant of the neurostimulation system 1202, a physician reviews patient data and assesses performance of the automatically programmed detection tools. The physician can finalize the detection tools by accepting the automatically programmed detection tools, or alternatively, by adjusting one or more detection settings to obtain desired performance. Upon finalization of the detection tools by the physician, the physician discontinues future automatic proposal and optimization of detection tools by the PDMS 1208, and enables stimulation therapy.

To ensure patient safety, the two-stage method for automatic proposal and adjustment of settings for detection tools occurs during a post-operative period prior to therapy activation. Because stimulation therapy is not yet enabled, there is no risk to the patient because the detection tools that are automatically implemented and tuned only impact detection and recording. In other embodiments, as disclosed above, the two-stage method is performed concurrently with delivery of stimulation therapy on one or more candidate detectors of the neurostimulation system 1202 that are not coupled to the therapy subsystem 328. In such embodiments, patient safety is preserved because detections by the candidate detectors do not cause delivery of stimulation therapy and because the parameters of the active detectors that govern stimulation therapy are not modified by the operations of the detection proposer 202 on the candidate detectors. The patient's ongoing stimulation therapy therefore remains under the control of the existing active detectors throughout the determination and tuning of the candidate detectors, and any change to the detection tool that governs stimulation therapy occurs only at the time of a handover, after handover criteria evaluated by the detection proposer 202 have been satisfied.

### Stage 1 Operation:

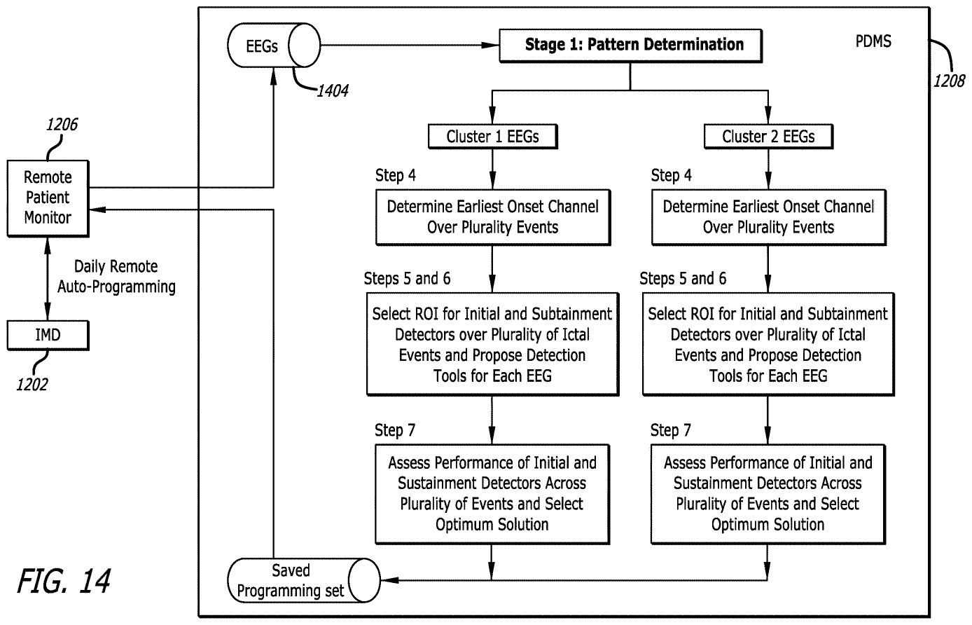

FIG. 14 is a flow chart for the first stage of the method of FIG. 13.

### Step 1: Gathering Patient Data (e.g., Ictal EEGs)

The neurostimulation system 1202 is programmed with default detection tools that respond to large power changes. The patient is discharged from the hospital and subsequently uses the remote patient monitor 1206 for days to weeks uploading patient data stored by the neurostimulation system 1202 to the PDMS 1208. The patient data includes EEG records 1404 of ictal events.

### Step 2: Seizure Classification and Onset Determination

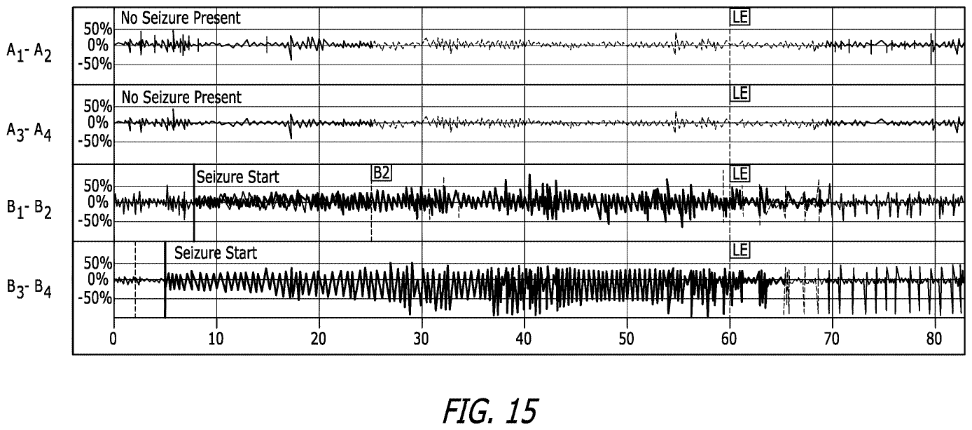

FIG. 15 is an illustration of an intracranial EEG record containing a seizure on two of four channels. In some embodiments, the PDMS 1208 includes a deep learning model such as a Vision Transformer (ViT) trained to assess which EEGs records 1404 are ictal on a channel-by-channel basis. The model also determines the seizure onset time of the seizure on each channel that has a seizure. In some embodiments, the PDMS 1208 includes the detection proposer 202 disclosed herein, which has an ESC module 501 configured to classify EEG records as seizure or non-seizure records and an EO/AT module 505 configured to determine seizure onset times.

In some embodiments, the EEG records 1404 that are classified as ictal records are clustered. Clustering EEG records classified as seizure records involves grouping similar seizure events based on signal characteristics such as frequency, amplitude, and spatial distribution. This process can identify different types of seizures that may need different detection tools, each tuned for reliable performance.

### Step 3: Clustering of Ictal Patterns in Reduced Dimension Space

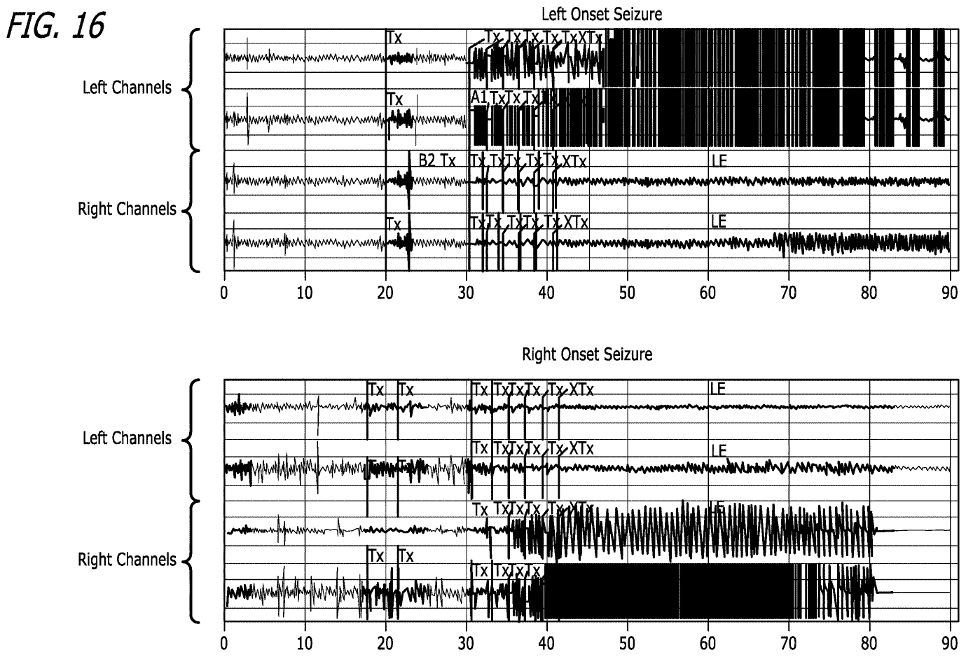

FIG. 16 shows EEG records for two seizures from one patient with a lead implanted in the left temporal lobe and another lead implanted in the right temporal lobes. In the top EEG record the seizure is on the left side, whereas in the bottom EEG record the seizure is on the right side. FIG. 17 is an illustration of an output of an exemplary clustering algorithm in reduced dimensional space. If the patient experiences a plurality of left and right events these can be represented in reduced dimension space and will cluster separately due to their different signal characteristics as shown in FIG. 17. Because each cluster represents different seizure patterns, a first detection tool is determined for purposes of detecting electrographic activity associated with the left-side cluster of EEGs and a second detection tool is determined for purposes of detecting electrographic activity associated with the right-side cluster of EEGs.

### Step 4: Determine Optimum Detection Channel for Each Seizure Cluster

In this step the earliest onset channel is determined over the plurality of ictal events within a cluster either using the mean or median onset times on each channel. The channel with the earliest onset time across the plurality of seizures is set as the detection channel for that cluster.

### Step 5: Automatically Determine Initial Detection Settings for Each Cluster

FIG. 18 is an illustration of an EEG record in which a ROI has been identified for proposal of detection settings for an initial detection tool. For each ictal EEG on the channel identified as the best detection channel in step 3, the seizure onset time determined by the deep learning model may be used to set the start time for a region of interest (ROI) to be analyzed for purposes of determining an initial detection tool. Hence if there are 8 ictal EEGs identified in the cluster, there may be 8 proposed solutions (one for each ictal EEG) on the same channel within each EEG. The length of the ROI varies by onset type as specified in Table 4.

In some embodiments, each ROI is analyzed to determine detection settings for the dominant pattern present within the ROI using methods disclosed herein with reference to FIGS. 8A-8D. In some embodiments, each ROI is analyzed to determine detection settings for the dominant pattern present within the ROI using methods disclosed in U.S. Pat. No. 10,233,715, titled Methods and Systems for Automatically Identifying Detection Parameters for an Implantable Medical Device, which is incorporated by reference.

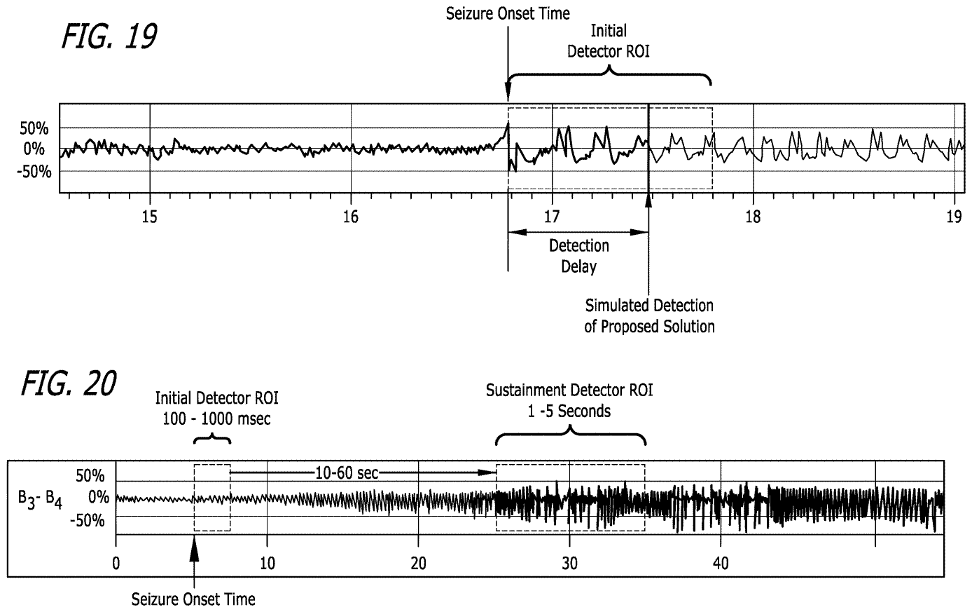

FIG. 19 is an illustration showing the performance of a proposed initial detection tool when applied to an example EEG record. The performance of each proposed initial detection tool may be evaluated against all the ictal EEGs records and the detection delay for each proposed initial detection tool across the plurality of ictal EEGs may be assessed. The proposed initial detection tool that has the earliest simulated detection time across the plurality of ictal EEGs (mean or median) may be selected as the selected (or final proposed) initial detection tool.

### Step 6: Automatically Determine Sustainment Detector Settings for Each Cluster

To trigger EEG storage of clinically significant events and/or act as a biomarker of seizure evolution, a sustainment detection tool is also determined. FIG. 20 is an illustration of an EEG record where sustainment ROI is identified for proposal of determining detection setting for a sustainment detection tool. In some embodiments the sustainment ROI begins on the order of 5-60 seconds after the initial ROI and is on the order of 1-5 seconds in duration.

In some embodiments, the sustainment ROI is analyzed to determine detection settings for a sustainment detection tool using methods disclosed herein with reference to FIGS. 8A-8D. In some embodiments the sustainment ROI is analyzed to determine detection settings for a sustainment detection tool using the methods disclosed in U.S. Pat. No. 10,233,715.

### Step 7: Assess Performance of Detection Tools

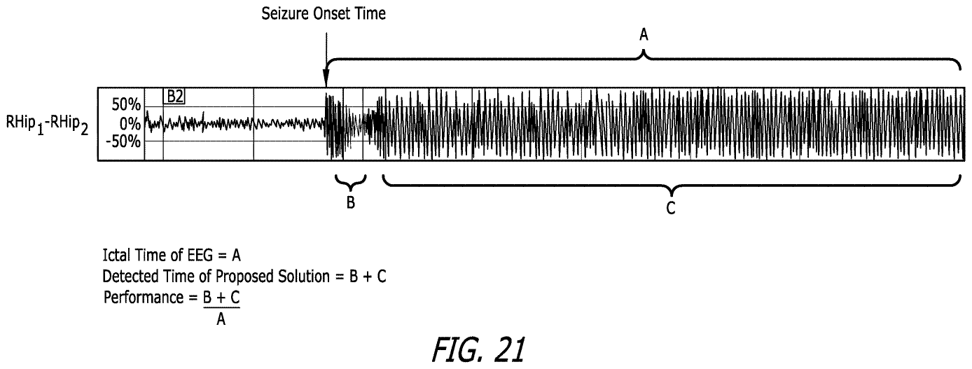

FIG. 21 is an illustration of an example metric for assessing performance of detection tool pair comprising an initial detection tool and a sustainment detection tool, when applied to an EEG record. The performance may be defined by the percent of time the proposed detection tool pair detect ictal activity relative to the total ictal time of EEG. The performance may be evaluated for a plurality of the ictal EEGs. The detection tool pair that has the performance closest to 1 across the plurality of ictal EEGs within specified variance criteria may be selected as the final detection tool pair.

### Stage 2 Operation: Sensitivity Titration

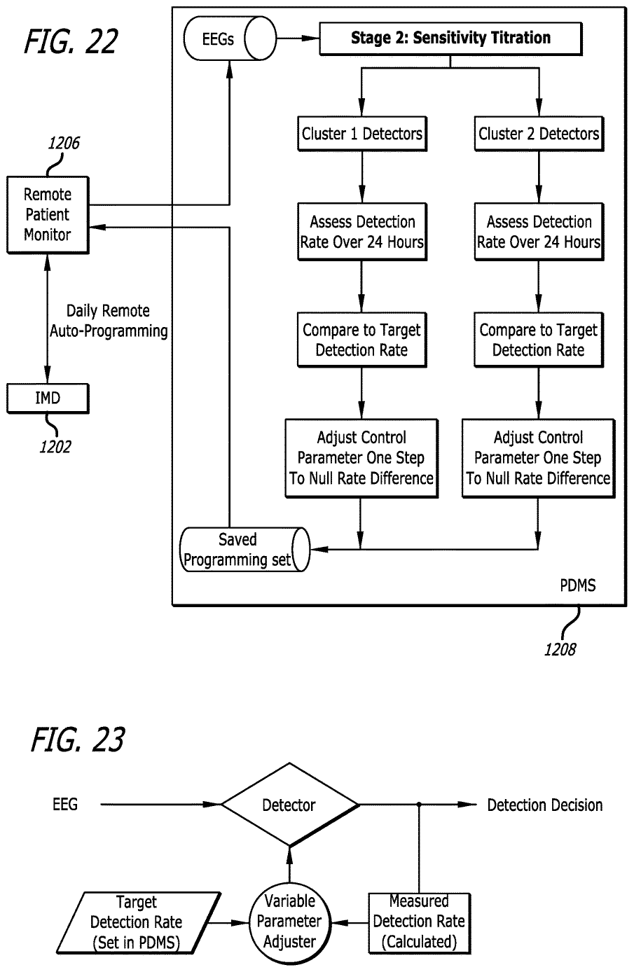

FIG. 22 is a flow chart for the second stage of the method of FIG. 13, during which settings or parameters for detection tools are incrementally adjusted, e.g., titrated, to obtain a desired metric, e.g., detection sensitivity. The goal of Stage 2 is to determine the optimum parameters to achieve a prescribed target detection rate based on the general detection parameters determined in Stage 1. Example methods of achieving target detection rate are described in U.S. Pat. No. 8,131,352, title System and Method for Automatically Adjusting Detection Thresholds in a Feedback-Controlled Neurological Event Detector, which is incorporated by reference. Other methods are possible.

In Stage 2, a detection setting or parameter of an initial detection tool and/or sustainment detection tool is selected as a control parameter. The control parameter is adjusted periodically, for example every 1-5 days. During the time between each control-parameter adjustment, electrical activity of the brain sensed by the implanted neurostimulation system is processed using the adjusted initial detection tool to detect for the respective pattern in electrographic activity that the adjusted initial detection tool is configured to detect. A detection performance metric, e.g., the detection rate, is determined and compared to the target detection rate for the initial detection tool. Subsequent adjustments to the control parameter are made to continue to minimize the difference between the actual detection rate and the target detection rate over periodic intervals (e.g., every 1-5 days).

Similarly, a detection setting or parameter of a sustainment detection tool is selected as a control parameter. The control parameter is adjusted periodically, for example every 1-5 days. During the time between each control-parameter adjustment, electrical activity of the brain sensed by the implanted neurostimulation system is processed using the adjusted sustainment detection tool to detect for the respective electrographic activity that the adjusted sustainment detection tool is configured to detect. A detection performance metric, e.g., the detection rate, is determined and compared to the target detection rate for the sustainment detection tool. Subsequent adjustments to the control parameter are made to continue to minimize the difference between the actual detection rate and the target detection rate over periodic intervals (e.g., every 1-5 days).

For neuromodulation systems similar to those described in U.S. Pat. No. 6,810,258, the available detector tools include bandpass detectors that detect activity within a specified band that exceeds a specified amplitude and duration criteria, and trending detection tools that use percentage changes in line length or area under the curve of the waveform in short-term windows compared to long-term trends. Once initial detection parameters are specified, some parameters (such as those in listed in Tables 6 and 7, below) can be used as control parameters with adjustment to these parameters having known effect on the detection rate, albeit in some cases the magnitude of the impact may only be ascertained by measuring the rate prospectively.

TABLE 6
(Bandpass Detection Tool)

| | Increase Detection | Decrease Detection |
|---|---|---|
| | Rate | Rate |
| Amplitude (minimum half- | Decrease value | Increase value |
| wave amplitude) | | |
| Duration (qualified analysis | Decrease value | Increase value |
| window count* | | |

* Note that qualified analysis window count and detection analysis window size together form the XofY criterion for bandpass detection. Decreasing the qualified analysis window count decreases X in XofY and increases detection sensitivity.

TABLE 7
(Line Length or Area Detection Tool)

| | Increase Detection | Decrease Detection |
|---|---|---|
| | Rate | Rate |
| Percentage Change | Decrease value | Increase value |
| (detection threshold) | | |
| Short Term Window (short- | Decrease value | Increase value |
| term window size) | | |

FIG. 23 is an illustration of an algorithm for adjusting settings of a detection tool in order to minimize the difference between an observed detection rate and a target detection rate. By starting with a baseline set of parameters defined in Stage 1 and then adjusting control parameters periodically (e.g., every 1-5 days), the difference between the actual detection rates and the target detection rates can be nulled out.

A method and/or system as described in this disclosure may include adjustment of stimulation settings, or other neurostimulator settings, in addition to or in place of adjustment of detection settings. The outcome measure for optimization may be one or more measures in place or in addition to detection rate; for example, amplitude of electrographic activity in one or more frequency bands, seizure counts, measures of autonomic tone, actigraphy, or symptomatology. A method and/or system as described in this disclosure may use an active medical device other than a neurostimulator, or a non-invasive neurostimulator, in addition to or in place of an implantable neurostimulator. A method and/or system as described in this disclosure may be applied to treatment of other neurological disorders or other non-neurological disorders (for example, cardiac rhythm disorders). In an embodiment of the invention, no physician programmer is required, or the physician programmer does not actively participate in the automatic adjustment of settings; in some embodiments, other components (such as one or more wearable sensors, or a patient's mobile phone) participate in the automatic adjustment of settings, for instance by collecting sensor data and providing it to the patient data management system. In some embodiments, the determination of when to invoke any operation described herein, including without limitation when to initiate the adaptive phase, when to initiate concurrent tuning of one or more candidate detectors, when to initiate a handover, and when to generate a notification of a readiness condition, is made automatically by the detection proposer 202, by another automated system or artificial intelligence or machine learning model in communication with the PDMS 222, by a human reviewer or physician, or by any combination thereof.

In some embodiments, one or more of the operations of the detection proposer 202, including without limitation any of the operations of the EEG records selection module 502, the detection window module 504, the candidate detection tool module 506, and the detection tool selection module 508, are performed by the IMD 206. In some embodiments, one or more of those operations are performed by the physician programmer 216. In some embodiments, one or more of those operations are performed by the remote patient monitor 210, 1206. In some embodiments, one or more of those operations are performed by the PDMS 222. In some embodiments, one or more of those operations are performed by another device, e.g., a workstation, a server, a clinician-facing computing device, or a patient-facing computing device, in communication with the IMD 206 or the PDMS 222 through the network 208. In some embodiments, the operations of the detection proposer 202 are partitioned across two or more of the IMD 206, the physician programmer 216, the remote patient monitor 210, 1206, the PDMS 222, and another such device, with each device performing a respective subset of the operations and providing the results of those operations to one or more of the other devices for further processing or programming. In some embodiments, the partition of operations is fixed; in other embodiments, the partition is dynamically determined based on factors such as available compute, available storage, available power, network connectivity, latency requirements, and patient privacy considerations.

In some embodiments, the primary metric used to determine whether a target performance metric is satisfied for a detection tool comprises one or more outcome measures other than, or in addition to, detection rate and detection lag. Examples of outcome measures include, without limitation: a count or rate of clinical seizures derived from a patient seizure diary, the seizure diary being received by the PDMS 222 through the remote patient monitor 210, 1206, a patient-facing application on a patient's mobile device, or another patient-reported channel; a count or rate of clinical seizures derived from a wearable sensor configured to sense one or more of motion, heart rate variability, electrodermal activity, peripheral electromyography, peripheral electrocardiogram other physiological correlate of seizure activity, the wearable sensor in communication with the PDMS 222 directly or through the IMD 206 or the remote patient monitor 210, 1206; one or more measures of autonomic tone derived from sensor data, e.g., heart rate variability or skin conductance; a measure of actigraphy or activity level derived from accelerometer data; a measure of sleep quality or sleep stage; a patient-reported symptomatology score; a quality-of-life score derived from one or more patient questionnaires; a medication adherence measure; and any combination of the foregoing. In each such embodiment, the detection proposer 202 obtains the outcome measure for a period of time during which a detection tool has been programmed into the IMD 206, compares the outcome measure to a target outcome measure, and adjusts one or more detection parameters of the detection tool, one or more stimulation parameters of the therapy subsystem 328, or both, to drive the outcome measure toward the target outcome measure. The adjustment can be performed using any of the coarse adjustment, titration, or in-situ adjustment processes described herein.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure, but are to be accorded the full scope consistent with the language of the claims.

What is claimed is:

1. A method comprising:
enabling an adaptive phase of an implanted neurostimulation system during which the implanted neurostimulation system refrains from delivering stimulation therapy;
during a first period of time of the adaptive phase, determining an initial detection tool configured to detect an onset pattern in electrographic activity sensed by a sensing channel of the implanted neurostimulation system, the initial detection tool defined by a set of detection parameters;
during a second period of time of the adaptive phase, performing at least one tuning cycle for the initial detection tool to determine a tuned initial detection tool by:
adjusting a detection parameter of the initial detection tool;
for a period of time subsequent to the adjustment of the detection parameter, detecting for the onset pattern in electrical activity using the adjusted initial detection tool, where the electrical activity is sensed during the period using the implanted neurostimulation system;
obtaining a performance metric for the adjusted initial detection tool based on data that results from the detecting;
determining if the performance metric satisfies a target performance metric;
responsive to the target performance metric not being satisfied, performing another tuning cycle; and
responsive to the target performance metric being satisfied, providing configuration information that defines the tuned initial detection tool; and
enabling a therapy phase of the implanted neurostimulation system during which neurostimulation therapy is delivered in response to a detection of the onset pattern by the tuned initial detection tool.

2. The method of claim 1, wherein adjusting the detection parameter comprises incrementally increasing or decreasing the detection parameter during each cycle based on prior adjustments and corresponding changes in performance metrics.

3. The method of claim 1, wherein the data that results from the detecting by the adjusted initial detection tool is a count of detections by the adjusted initial detection tool, and the performance metric is detection rate.

4. The method of claim 1, wherein the data that results from the detecting by the adjusted initial detection tool comprises an EEG record of the electrical activity of a brain and the performance metric is a lag between an onset of the pattern in the EEG record as detected by the adjusted initial detection tool, and an actual onset of the pattern in the EEG record.

5. The method of claim 1, further comprising:
during the first period of time of the adaptive phase, determining a sustainment detection tool configured to detect a sustainment pattern indicative of electrographic activity that can follow the onset pattern in electrographic activity; and
during the second period of time of the adaptive phase, tuning the sustainment detection tool by:
adjusting a detection parameter of the sustainment detection tool;
for a period of time subsequent to the adjustment of the detection parameter, detecting for the sustainment pattern in electrical activity using the adjusted sustainment detection tool, where the electrical activity is sensed during the period using the implanted neurostimulation system;
obtaining a performance metric for the adjusted sustainment detection tool based on data that results from the detecting;
determining if the performance metric satisfies a target performance metric; and
responsive to the target performance metric not being satisfied, repeating the adjusting, detecting, obtaining, and determining; and
responsive to the target performance metric being satisfied, enabling the implanted neurostimulation system to trigger a recording of electrical activity in response to a detection of the sustainment pattern by the tuned sustainment detection tool.

6. The method of claim 5, wherein adjusting the detection parameter comprises incrementally increasing or decreasing the detection parameter during each cycle based on prior adjustments and corresponding changes in performance metrics.

7. The method of claim 5, wherein the data that results from the detecting by the adjusted sustainment detection tool comprises an EEG record of the electrical activity of a brain and the performance metric is a function of the total time of detection by the sustainment detection tool relative to a total ictal time of the EEG record.

8. The method of claim 7, wherein the performance metric is further a function of a total time of detection by the adjusted initial detection tool.

9. The method of claim 5, wherein determining the sustainment detection tool comprises:
processing each of a plurality of sustainment regions of interest (SROI) of a selected EEG record to implement a corresponding plurality of candidate sustainment detection tools to detect the respective pattern indicative of electrographic activity that follows the onset of an electrographic seizure; and
processing the plurality of candidate sustainment detection tools to identify a selected sustainment detection tool.

10. The method of claim 9, further comprising:
identifying a set of EEG records with electrographic activity previously sensed by the sensing channel and indicative of a type of electrographic seizure; and
selecting the selected EEG record from the set of EEG records, wherein the selected EEG record satisfies a specified criterion.

11. The method of claim 10, wherein the specified criterion is selected from the group consisting of: earliest seizure onset time, a cluster centroid, a confidence level, a chronological criterion corresponding to one of earliest EEG record or latest EEG record, an EEG signal quality criterion, or a combination of two or more of these criteria.

12. A system for managing the configuration of an implanted neurostimulation system having a therapy phase of operation during which stimulation therapy is delivered, and an adaptive phase of operation during which stimulation therapy is not delivered, the system comprising:
a patient data management system configured to:
during a first period of the adaptive phase, obtain an initial detection tool configured to detect an onset pattern in electrographic activity sensed by a sensing channel of the implanted neurostimulation system, the initial detection tool defined by a set of detection parameters;
during a second period of the adaptive phase, perform at least one tuning cycle of the initial detection tool to determine a tuned initial detection tool by being further configured to:
adjust a detection parameter of the initial detection tool;
receive for a period of time subsequent to the adjustment of the detection parameter, data from the implanted neurostimulation system that results from detections of the onset pattern by the adjusted initial detection tool;
obtain a performance metric for the adjusted initial detection tool based on the data;
determine if the performance metric satisfies a target performance metric;
responsive to the target performance metric not being satisfied, perform another tuning cycle; and
responsive to the target performance metric being satisfied, provide configuration information that defines the tuned initial detection tool, where the provided configuration information corresponds to the configuration information of the adjusted initial detection tool that satisfied the target performance metric.

13. The system of claim 12, wherein the data that results from the detecting by the adjusted initial detection tool is a count of detections by the adjusted initial detection tool, and the performance metric is detection rate.

14. The system of claim 12, wherein the data that results from the detecting by the adjusted initial detection tool comprises an EEG record of the electrical activity of a brain and the performance metric is a lag between an onset of the pattern in the EEG record as detected by the adjusted initial detection tool, and an actual onset of the pattern in the EEG record.

15. The system of claim 12, wherein the patient data management system provides the configuration information that defines the tuned initial detection tool by being configured to program the tuned initial detection tool into the implanted neurostimulation system based on the configuration information.

16. The system of claim 12, further comprising a physician programmer, wherein:
the patient data management system provides the configuration information that defines the tuned initial detection tool by being configured to provide the configuration information to the physician programmer; and
the programmer is configured to program the tuned initial detection tool into the implanted neurostimulation system based on the configuration information.

17. The system of claim 12, wherein the patient data management system obtains the initial detection tool by being configured to receive configuration information that defines the initial detection tool.

18. The system of claim 12, wherein the patient data management system is further configured to:
during the first period of the adaptive phase, obtain a sustainment detection tool configured to detect a sustainment pattern indicative of electrographic activity that can follow the onset pattern in electrographic activity; and
during the second period of time of the adaptive phase, perform at least one tuning cycle of the sustainment detection tool to determine a tuned sustainment detection tool by being further configured to:
adjust a detection parameter of the sustainment detection tool;
receive for a period of time subsequent to the adjustment of the detection parameter, data from the implanted neurostimulation system that results from detections of the sustainment pattern by the adjusted sustainment detection tool;
obtain a performance metric for the adjusted sustainment detection tool based on the data;
determine if the performance metric satisfies a target performance metric;
responsive to the target performance metric not being satisfied, perform another tuning cycle; and
responsive to the target performance metric being satisfied, provide configuration information that defines the tuned sustainment detection tool, where the provided configuration information corresponds to the configuration information of the adjusted sustainment detection tool that satisfied the target performance metric.

19. The system of claim 18, wherein the patient data management system obtains the sustainment detection tool by being further configured to:
process each of a plurality of sustainment regions of interest (SROI) of a selected EEG record to implement a corresponding plurality of candidate sustainment detection tools to detect the respective pattern indicative of electrographic activity that follows the onset of an electrographic seizure; and
process the plurality of candidate sustainment detection tools to identify a selected sustainment detection tool.

20. The system of claim 19, wherein the patient data management system is further configured to:
identify a set of EEG records with electrographic activity previously sensed by the sensing channel and indicative of a type of electrographic seizure; and
select the selected EEG record from the set of EEG records, wherein the selected EEG record satisfies a specified criterion.

21. The system of claim 20, wherein the specified criterion is earliest seizure onset time, and the patient data management system selects the EEG record by being further configured to apply the set of EEG records to a machine learning based model configured to identify the EEG record with the earliest seizure onset time.

22. The system of claim 20, wherein the specified criterion is a cluster centroid and the patient data management system selects the EEG record by being further configured to apply the set of EEG records to a machine learning based model comprising a clustering model configured to identify the EEG record that is nearest to the cluster centroid.

23. The system of claim 20, wherein the specified criterion is a confidence level and the patient data management system selects the EEG record by being further configured to apply the set of EEG records to a machine learning based model configured to identify the EEG record with the highest confidence level.

24. The system of claim 20, wherein the specified criterion is a chronological criterion corresponding to one of: earliest EEG record or latest EEG record.

25. The system of claim 20, wherein the specified criterion is an EEG signal quality criterion.

26. The system of claim 18, wherein the patient data management system obtains the sustainment detection tool by being configured to receive configuration information that defines the sustainment detection tool.

27. The system of claim 18, wherein the patient data management system provides the configuration information that defines the tuned sustainment detection tool by being configured to program the tuned sustainment detection tool into the implanted neurostimulation system based on the configuration information.

28. The system of claim 18, further comprising a physician programmer, wherein:
the patient data management system provides the configuration information that defines the tuned sustainment detection tool by being configured to provide the configuration information to the physician programmer; and
the programmer is configured to program the tuned sustainment detection tool into the implanted neurostimulation system based on the configuration information.
